(19) United States
(12) Patent Application Publication
(10) Pub. No.: US 2015/0258096 A1
(43) Pub. Date: Sep. 17, 2015

(54) METHODS AND COMPOSITIONS FOR TREATMENT OF TH2-MEDIATED AND TH17-MEDIATED DISEASES

(71) Applicant: THE REGENTS OF THE UNIVERSITY OF CALIFORNIA, Oakland, CA (US)

(72) Inventors: Eyal Raz, Del Mar, CA (US); Xiangli Li, San Diego, CA (US); Jihyung Lee, San Diego, CA (US); Paul A. Insel, La Jolla, CA (US); Fiona Murray, San Diego, CA (US); Taehun Kim, San Diego, CA (US)

(21) Appl. No.: 14/434,947

(22) PCT Filed: Oct. 10, 2013
(86) PCT No.: PCT/US2013/064342

§ 371 Date: Apr. 10, 2015

Related U.S. Application Data
(60) Provisional application No. 61/712,154, filed on Oct. 10, 2012.
(60) Provisional application No. 61/824,543, filed on May 17, 2013.

(51) Int. Cl. A61K 31/522; A01K 67/027; A61K 31/352

(52) U.S. Cl. CPC A61K 31/522; A61K 31/352; A01K 67/0276; A01K 2227/105; A01K 2267/0387

(57) ABSTRACT

Provided herein, inter alia, are methods drawn to treatment of Th2-mediated and Th17-mediated diseases. Also provided herein is a mouse model that develops Th2 responses to environmental stimuli in a similar manner as human subjects.

### CROSS-REFERENCES TO RELATED APPLICATIONS

This application claims priority to U.S. Provisional Application No. 61/712,154, filed Oct. 10, 2012 and to U.S. Provisional Application No. 61/824,543, filed May 17, 2013.

### STATEMENT AS TO RIGHTS TO INVENTIONS MADE UNDER FEDERALLY SPONSORED RESEARCH AND DEVELOPMENT

This invention was made with Government support under grant numbers AI095623, DK035108, AI077989 (ER), awarded by National Institutes of Health. The Government may have certain rights in this invention.

### BACKGROUND OF THE INVENTION

The increasing prevalence of allergic diseases in developed and developing countries over the last few decades imposes significant public health challenges. Food allergy and atopic dermatitis generally occur in the first year of life, followed by allergic rhino-conjunctivitis and then, by allergic asthma. The prevalence of allergic diseases in the general population is 20% with an estimated health care related cost of $20 billion/year. Many allergic diseases are provoked by Th2 responses to allergens. However, many therapies fail clinically because of a lack of efficacy and/or safety. Thus, the failure to translate promising drug candidates to humans questions the utility of present animal studies and demands more predictive models that reflect human genetics and immunology. There is a need for predictive models to reflect human genetics and immunology with respect to Th2 induced allergies and disease. Provided herein are solutions to these and other problems in the art.

### BRIEF SUMMARY OF THE INVENTION

Accordingly, provided herein, inter alia, are methods drawn to treatment of Th2-mediated and Th17-mediated diseases. Also provided herein is a mouse model that develops Th2 responses to environmental stimuli in a similar manner as human subjects.

In a first aspect is a method of inhibiting dendritic cell induction of CD4 T cell lineage conversion to a Th2 cell. The method includes contacting a dendritic cell with a cAMP-elevating agent in the presence of a CD4 T cell. The cAMP concentration within said dendritic cell is allowed to increase relative to the absence of the cAMP-elevating agent thereby inhibiting dendritic cell induction of lineage conversion of the CD4 T cell to a Th2 cell. The cAMP-elevating agent is exogenous to said dendritic cell

In another aspect is a method of activating dendritic cell induction of CD4 T cell lineage conversion to a Th2 cell. The method includes contacting a dendritic cell with a cAMP-lowering agent in the presence of a CD4 T cell. The cAMP concentration within the dendritic cell is allowed to decrease relative to the absence of the cAMP-lowering agent thereby activating dendritic cell induction of lineage conversion of the CD4 T cell to a Th2 cell.

In another aspect is a method of treating a Th2-mediated disease in a patient in need thereof. The method includes administering to the patient an effective amount of a cAMP-elevating agent.

In another aspect is a method for treating a Th2-mediated disease in a patient in need thereof. The method includes administering to the patient an effective amount of a cAMP-lowering agent.

In another aspect is a method for treating a Th17-mediated disease in a patient in need thereof. The method includes administering to the patient an effective amount of a cAMP-lowering agent.

In another aspect is a method of preventing a Th2-mediated disease in a patient in need thereof. The method includes administering to the patient an effective amount of a cAMP-elevating agent in combination with an adjuvant.

In another aspect is a method for preventing a Th17-mediated disease in a patient in need thereof. The method includes administering to the patient an effective amount of a cAMP-lowering agent in combination with an adjuvant.

In another aspect is a method of inducing CD4 T cell lineage conversion using an APC. The method includes contacting an APC with a cAMP-lowering agent. The cAMP-lowering agent is allowed to lower cAMP levels in the APC, thereby forming an activated-APC. The activated-APC is contacted with a first mature CD4 T cell. The activated-APC is allowed to convert the lineage of the first mature CD4 T cell into a second mature CD4 T cell, thereby inducing CD4 T cell lineage conversion using an APC.

In another aspect is a method of inducing CD4 T cell lineage conversion using an APC. The method includes contacting an APC with a cAMP-elevating agent. The cAMP-elevating agent is allowed to elevate cAMP levels in the APC, thereby forming an activated-APC. The activated-APC is contacted with a first mature CD4 T cell. The activated-APC is allowed to convert the lineage of the first mature CD4 T cell into a second mature CD4 T cell, thereby inducing CD4 T cell lineage conversion using an APC.

In another aspect is a method of identifying a cAMP-elevating agent. The method includes contacting a test compound with an APC. The test compound is allowed to elevate cAMP levels in the APC thereby forming an activated-APC. An elevated level of cAMP in the activated-APC is detected thereby identifying a cAMP-elevating agent.

In another aspect is a method of identifying a cAMP-lowering agent. The method includes contacting a test compound with an APC. The test compound is allowed to lower cAMP levels in the APC thereby forming an activated-APC. A lowered level of cAMP in the activated-APC is detected thereby identifying a cAMP-lowering agent.

In another aspect is a method of identifying a cAMP-elevating agent in the presence of an adjuvant. The method includes contacting a test compound and an adjuvant with an APC. The test compound is absorbed or bound to the adjuvant and allowed to elevate cAMP levels in the APC thereby forming an activated-APC. An elevated level of cAMP in the activated-APC is detected thereby identifying a cAMP-elevating agent.

In another aspect is a method of identifying a cAMP-lowering agent in the presence of an adjuvant. The method includes contacting a test compound and an adjuvant with an APC. The test compound is absorbed or bound to the adjuvant and allowed to lower cAMP levels in the APC thereby forming an activated-APC. A lowered level of cAMP in the activated-APC is detected thereby identifying a cAMP-lowering agent.

In another aspect is a method of identifying a cAMP-elevating agent in an APC Gαs-knockout mouse. The method includes administering a test compound to a Gαs-knockout mouse. The test compound is allowed to elevate cAMP levels in the Gαs-knockout mouse. The elevated cAMP levels in the Gαs-knockout mouse are then detected.

In another aspect is a method of identifying a cAMP-lowering agent in an APC Gαs-knockout mouse. The method includes administering a test compound to a Gαs-knockout mouse. The test compound is allowed to lower cAMP levels in the Gαs-knockout mouse. The lowered cAMP levels in the Gαs-knockout mouse are then detected.

In another aspect is a method of treating a Th2-mediated disease in a patient in need thereof. The method includes detecting a cAMP level in a patient sample (e.g., for pharmacogenetic analysis). The cAMP level is compared to a control thereby identifying a low cAMP level in the patient sample. An effective amount of a cAMP-elevating agent is then administered to the patient thereby treating the Th2-mediated disease.

In another aspect is a method of treating a Th17-mediated disease in a patient in need thereof. The method includes detecting a cAMP level in a patient sample. The cAMP level is compared to a control thereby identifying a high cAMP level in the patient sample. An effective amount of a cAMP-lowering agent is then administered to the patient thereby treating the Th2-mediated disease.

In another aspect is a method of identifying a Th2-mediated disease in a patient. The symptoms of the Th2-mediated disease are similar to a Th17-mediated disease (e.g., bronchial asthma). The method includes detecting a cAMP level in a patient sample. The cAMP level is compared to a control thereby identifying a low cAMP level in the patient sample, and thereby identifying the Th2-mediated disease in a patient.

In another aspect is a method of identifying a Th17-mediated disease in a patient. The symptoms of the Th17-mediated disease are similar to a Th2-mediated disease. The method includes detecting a cAMP level in a patient sample. The cAMP level is compared to a control thereby identifying a high cAMP level in the patient sample, and thereby identifying the Th17-mediated disease in a patient.

In another aspect is a conditional Gαs-knockout mouse having dendritic cells with a Gas deletion.

In another aspect is a method of producing a Gαs-knockout mouse. The method includes crossing a lox-flanked Gnas mouse with a CD11c-Cre or LysM-Cre mouse, wherein the Gαs-knockout mouse does not express Gαs.

### BRIEF DESCRIPTION OF THE DRAWINGS

FIG. 1: Conditional deletion of Gnas in CD11⁺ cells impairs cAMP production: (a) CD11c-specific deletion of Gnas was confirmed by qPCR. Total mRNA was prepared from FACS-sorted splenic cells CD11c⁺CD11b⁻TCRβ⁻CD19⁻, (b) cAMP level was determined by RIA⁵⁰ in CD11c⁺ cells treated with vehicle, 10 μM forskolin (Fsk), 10 μM isoproterenol (Iso), or 1 μM prostaglandin E₂ (PGE₂) in the presence of the 200 μM PDE inhibitor IBMX, (c, d) Total mRNA and cAMP accumulation in the cells expressing CD11b⁺CD11c⁻TCRβ⁻CD19⁻ from fl/fl and Gnas^{ΔCD11c} mice (Data are mean±s.e.m. n=3/group, from a representative experiment; ** p<0.01).

FIG. 2: Immune development in Gnas^{ΔCD11c} mice is not affected by Gnas deletion: (a) The cell number and percentage of splenic CD11c⁺ cells and the percentage of splenic total CD4⁺, effector memory (CD44^{high}CD62^{low}) and naïve (CD44^{low}CD62L^{high}) CD4⁺, CD8⁺, and B220⁺ cells, respectively, in 2 month-old Gnas^{ΔCD11c} and fl/fl mice (FACS), (b) The expression of costimulatory molecules in CD11c⁺ cells from 2 month-old fl/fl and Gnas^{ΔCD11c} mice were measured by FACS, (c) Cytokine profile of anti-CD3/28 Ab stimulated CD4⁺ T cells (spleen) from 2-month old fl/fl and Gnas^{ΔCD11c} mice (ELISA), (d) Intact histological analysis of lung tissue in 2-month old fl/fl and Gnas^{ΔCD11c} mice H &E, PAS, trichrome, and anti-SM actin staining are shown (magnification ×100, scale bar: 100 μm) (The data shown are one of three independent experiments with similar results).

FIG. 3: Gnas^{ΔCD11c} mice are atopic and are predisposed toward Th2 immunity: (a) Serum IgE, IgG1, and IgA levels in the 2-month old fl/fl and Gnas^{ΔCD11c} mice (ELISA), IgG2a levels were below the detection level, (b) OVA immunization protocol and challenge, (c) Mean values±s.e.m. of airway resistance for fl/fl and Gnas^{ΔCD11c} mice after intranasal (i.n) OVA instillation and methacholine (MCh) challenge, (d) Total cell and (e) eosinophil counts in bronchoalveolar lavage (BAL) fluid, Cytokine response of CD4⁺ T cells from the (f) bronchial lymph nodes and (g) spleen, (h) H&E staining of the lung (magnification ×100, scale bar: 100 μm) (Data are mean±s.e.m., n=4-6 in each group; * p<0.05, ** p<0.01, p<0.001).

FIG. 4: Spontaneous Th2 responses in 6-month old Gnas^{ΔCD11c} mice: (a) Cytokine profile of anti-CD3/28 Ab-stimulated CD4⁺ T cells (spleen) from 6-month old fl/fl and Gnas^{ΔCD11c} mice (ELISA), (b) Mean values±s.e.m. of airway resistance after MCh challenge, (c) Total cell and eosinophil counts in the BAL fluid, (d) Serum IgE, IgG1, and IgA levels (ELISA), (e) Histologic lung tissue analysis: H&E, PAS (red-purple), Trichrome (blue) and anti-SMA (brown) in the lung tissues (magnification ×100, scale bar: 100 μm) (Data are mean±s.e.m, n=4-6 in each group; * p<0.05, ** p<0.01, p<0.001).

FIG. 5: Housing conditions determine allergic inflammation in the lung of Gnas^{ΔCD11c} mice: (a) Cytokine profile of anti-CD3/28 Ab stimulated CD4⁺ T cells (spleen) from 6-month old fl/fl and Gnas^{ΔCD11c} mice under SPF conditions (ELISA), (b) Total cell and eosinophil counts in the BAL fluid, (c) Histological lung evaluation: H&E, PAS, trichrome, and anti-SM actin staining, (d) Serum IgE, IgG1, and IgA levels (ELISA) (Data are mean±s.e.m, n=4-6 in each group; * p<0.05).

FIG. 6: BMDC from Gnas^{ΔCD11c} mice induce a Th2 bias: FACS-sorted CD11c⁺CD135⁺BM cells from fl/fl and Gnas^{ΔCD11c} mice (5×10⁵ cells per condition) were then co-cultured with naïve FACS-sorted OT-2 CD4⁺ T cells (1:1 ratio) for 3 days and then stimulated with plate-bound anti-CD3/28 Abs; (a) cytokines levels (ELISA), (b) intracellular cytokine staining (FACS), (c) levels of co-stimulatory molecules (FACS), and (d) qPCR analysis of lineage commitment factors in the isolated OT-2 CD4⁺ T cells. (e) Naïve IL4-eGFP reporter (4get) CD4⁺ T cells (2×10⁶ dells/mouse) were i.v. transferred into RAG KO (red) or RAG/Gnas^{ΔCD11c} DKO (blue) mice—the eGFP fluorescence intensity of the splenic TCRβ⁺ cells was recorded (FACS) (Data are mean±s.e.m, n=4-6 in each group; ** p<0.01. NS-non-significant).

FIG. 7: CD11c⁺ BM cells from Gnas^{ΔCD11c} mice induce a Th2 bias: (a) Composition of CD11c⁺ CD135⁺ cells from fl/fl and Gnas^{ΔCD11c} mice, FACS-sorted CD11c⁺CD135⁻ cells from fl/fl and Gnas^{ΔCD11c} mice were co-cultured with naïve OT2 CD4⁺ T cells for 3 days and then stimulated with plate-bound anti-CD3/28 Abs, after which (b) cytokines levels (ELISA) and (c) intracellular cytokine staining (FACS), and (d) qPCR analysis of lineage commitment factors of the isolated OT2 cells were determined (Data are mean±s.e.m, n=4-6 in each group; ** p<0.01).

FIG. 8: Flt3 ligand-stimulated BM cells induce Th2 differentiation: BM cell were cultured in the presence of Flt3 ligand for 10 days, washed and then co-cultured with naïve OT2 CD4⁺ T cells for 3 days (1:1 ratio), OT2 CD4⁺ T cells were isolated and stimulated with plate-bound anti-CD3/28 Abs, after which cytokines levels were analyzed (ELISA) (Data are mean±s.e.m, n=4-6 in each group; * p<0.05, ** p<0.01).

FIG. 9: Analysis of cAMP signaling and genes involved in the pro-Th2 DC phenotype: IL-4 levels of anti-CD3/28 Ab-stimulated OT-2 CD4⁺ T cells co-cultured with (a) CD11c⁺ BM cells from fl/fl and Gnas^{ΔCD11c} mice treated with N6 (a PKA-specific cAMP analogue, 50 μM) or 8ME (an EPAC-specific cAMP analogue, 50 μM) (ELISA), (b) WT (B6) CD11c⁺ BM cells treated with EPAC inhibitor (CE3F4, 50 μM) or PKA inhibitor (H-89, 10 μM) with or without PTX (100 μg/ml) (ELISA), (c) WT CD11c⁺ BM cells treated with MP7 (1 μM) with or without PTX (100 μg/ml) (ELISA), (d) Gnas^{ΔCD11c} CD11c⁺ BM cells treated with PTX (100 μg/ml), (e) Scatterplot showing log 2-normalized levels of genes expressed by CD11c⁺ BM cells generated from Gnas^{ΔCD11c} and fl/fl mice, (f) Table listing mouse genes with altered expression in Gnas^{ΔCD11c} CD11c⁺ BM cells (p-value) that are also human GWAS allergy/asthma susceptibility genes (Up-regulated genes are shown in bold print and down-regulated genes in regular print), (g) The mRNA levels (qPCR) of CCL2 in fl/fl and Gnas^{ΔCD11c} CD11c⁺ BM cells incubated without or with 8-CPT-cAMP (50 μM), (h) IL-4 levels of anti-CD3/28 Ab-stimulated OT-2 CD4⁺ T cells co-cultured with CD11c⁺ BM cells treated with anti-CCL2 neutralizing Abs. (ELISA) (Data are mean±s.e.m, n=3 in each group; * p<0.05, ** p<0.01, p<0.001).

FIG. 10: CREB1-cebntric transcription factor network: The 717 genes with >2-fold change in expression in Gnas^{ΔCD11c} CD11c⁺ BM cells were analyzed for their transcription factor regulation using Metacore, the top network containing 208 genes centering on CREB1 is shown; genes with increased expression are indicated by a dot, genes with decreased expression by a dot. Arrows indicate, respectively, stimulatory, inhibitory and undefined interactions.

FIG. 11: Highest ranking human asthma gene set enriched in WT CD11c⁺ BM cells: Left panel: Enrichment Score in green is plotted for the ranked list of genes—Mouse genes are ranked based on the correlation between their expression and the genotype. Gray indicates mouse genes that correlate with fl/fl (WT) cells and black with Gnas^{ΔCD11c} CD11c⁺ BM cells; the genes in the target human gene set are indicated by vertical lines. Enrichment Score reflects the degree to which a gene set is overrepresented at the top or bottom of the ranked list of mouse genes shown at bottom: Right panel: Heatmap of the genes in this geneset where gray indicates increased expression and black indicates decreased expression for two fl/fl and two Gnas^{ΔCD11c} samples (The gene symbol and gene description are shown to the right of the heatmap).

FIG. 12: Highest ranking human atopy gene set enriched in WT CD11c⁺ BM cells: Left panel: Enrichment score in green is plotted for the ranked list of genes with the geneset genes indicated by vertical lines; Right panel: Heatmap of the genes in this geneset where gray indicates increased expression and black indicates decreased expression for two fl/fl and two Gnas^{ΔCD11c} samples (The gene symbol and gene description are shown to the right of the heatmap).

FIG. 13: Highest ranking human asthma geneset enriched in Gnas^{ΔCD11c} BM CD11c⁺ cells: Left panel: Enrichment score in green is plotted for the ranked list of genes with the geneset genes indicated by vertical lines; Right panel: Heatmap of the genes in this geneset where gray indicates increased expression and black indicates decreased expression for two fl/fl and two Gnas^{ΔCD11c} samples (The gene symbol and gene description are shown to the right of the heatmap).

FIG. 14: Adoptive transfer of CD11c⁺ BM cells from Gnas^{ΔCD11c} mice induces a Th2 bias in vivo, a response that is inhibited by a cell-permeable cAMP analogue: (a) OVA-specific IL-4 response by OT-2 CD4⁺ T cells co-cultured with cell-permeable cAMP (8-CPT-cAMP, 50 μM)-treated CD11c⁺ BM cells, (b) Protocol of the adoptive transfer. OVA-loaded Gnas^{ΔCD11c} CD11c⁺ BM cells were incubated in the absence and presence of 50 μM 8-CPT-cAMP (CPT) in vitro prior to i.n. transfer to WT (B6 mice) and Gnas^{ΔCD11c} recipients (2×10⁵ cells/recipient), (c) IL-4 levels of anti-CD3/28 Ab-stimulated CD4⁺ T cells (spleen) from WT or Gnas^{ΔCD11c} recipients (ELISA), (d) Serum levels of IgE and IgG1 from WT and Gnas^{ΔCD11c} mice that received Gnas^{ΔCD11c} CD11c⁺ BM cells loaded with OVA with/without CPT, (e) Lung histology from WT and Gnas^{ΔCD11c} recipients (magnification ×100, scale bar: 100 μm) (Data are mean±s.e.m, n=3-4 in each group; * p<0.05, ** p<0.01, p<0.001).

FIG. 15: Schematic of adoptive transfer of Gnas^{ΔCD11c} BM CD11c+ cells treated w/wo cell-permeable cAMP: BMDCs are derived from ΔCD11c mice as described herein and exposed to OVA and cAMP wherein the OVA-loaded BMDCs are transferred to WT or ΔCD11c mice and analyzed.

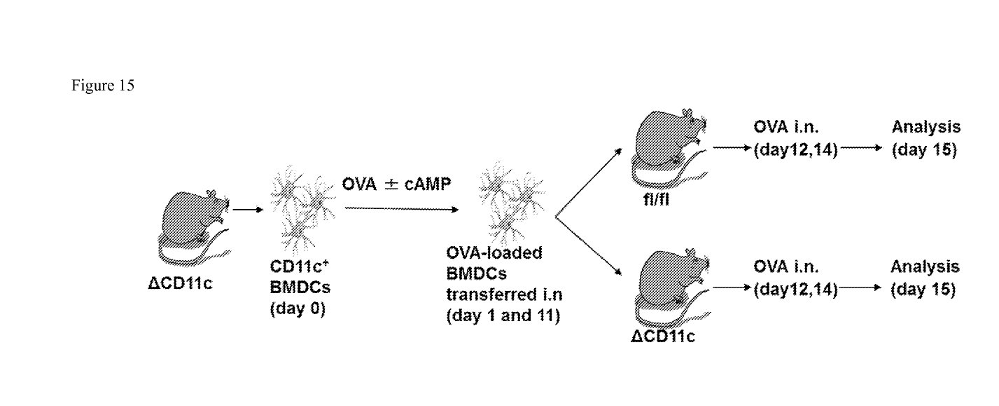

FIG. 16 cAMP agents provoke IL-17 responses: Wild-type B6 mice were immunized intraperitoneally (i.p.) twice two weeks apart with OVA (50 μg/mice) with and without alum (20 mg/mice), and colforsin (CF; a cAMP elevating drug that is approved for human use in Japan, 1 mg/kg), IBMX (a PDE inhibitor, 5 mg/kg), or solvent only as a control; on day 28, single-cell suspensions were prepared from the spleens and incubated for 3 days with OVA (200 μg/mL) as we described earlier (16); IL-17 levels were then detected (ELISA); *p<0.05 and **p<0.01 compared with OVA/alum-immunized group, n=4/group.

FIG. 17: Anti-OVA IgG titer in the sera of immunized mice. The anti-OVA IgG titer was measured in the sera of immunized mice (ELISA); ninety-six well plates were coated with 2 ug/ml of OVA and then blocked with 1% BSA PBS; Plates were washed and incubated with diluted serum for 2 h at RT and after thorough washing, bound IgG was detected by HRP-labeled goat anti-mouse IgG, followed by TMB substrate development; antibody (IgG) titers were determined by comparison to a standard curve generated using sera from OVA hyper-immunized mice, and were expressed as the reciprocal end point dilution (**p<0.01 compared with OVA/alum-immunized group, n=4/group).

FIG. 18: DC-specific drug discovery for potential interventions in Th2 and Th17-mediated diseases: Th17 and Th2 related diseases are mediated by the intracellular cAMP concentration which can be analyzed at multiple different levels starting at the GPCR level through a GPCR array, post GPCR signaling, targeting phagocytes, and functional genomics and test compounds.

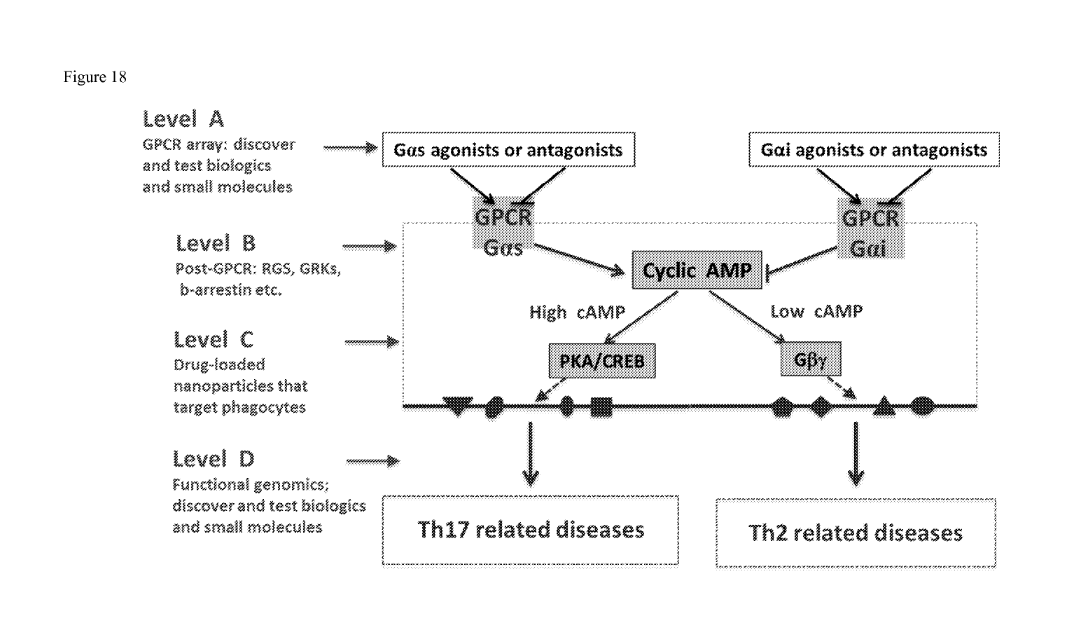

FIG. 19: Co-culture system: BMDC (GM-CSF) and OT2 CD4 T cells: BMDC exposed to OVA can be co-cultured with naïve OT2 T cells to analyze T cell responses from induction to Th subsets by the BMDC.

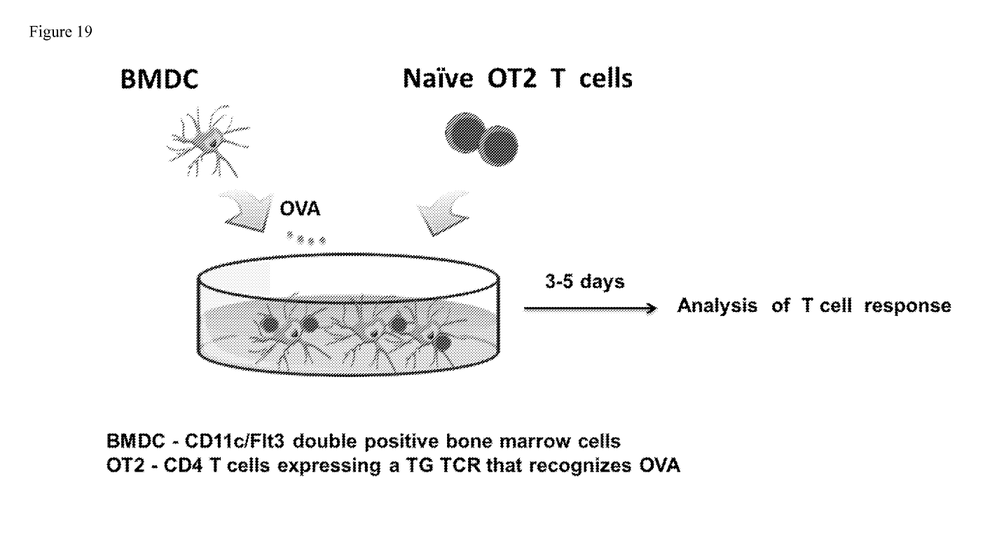

FIG. 20: Microarray analysis of GWAS in asthmatic patients: regulated genes match multiple genes found in asthmatic patients (*=match hGWAS in allergic asthma; **=match hGWAS in asthma).

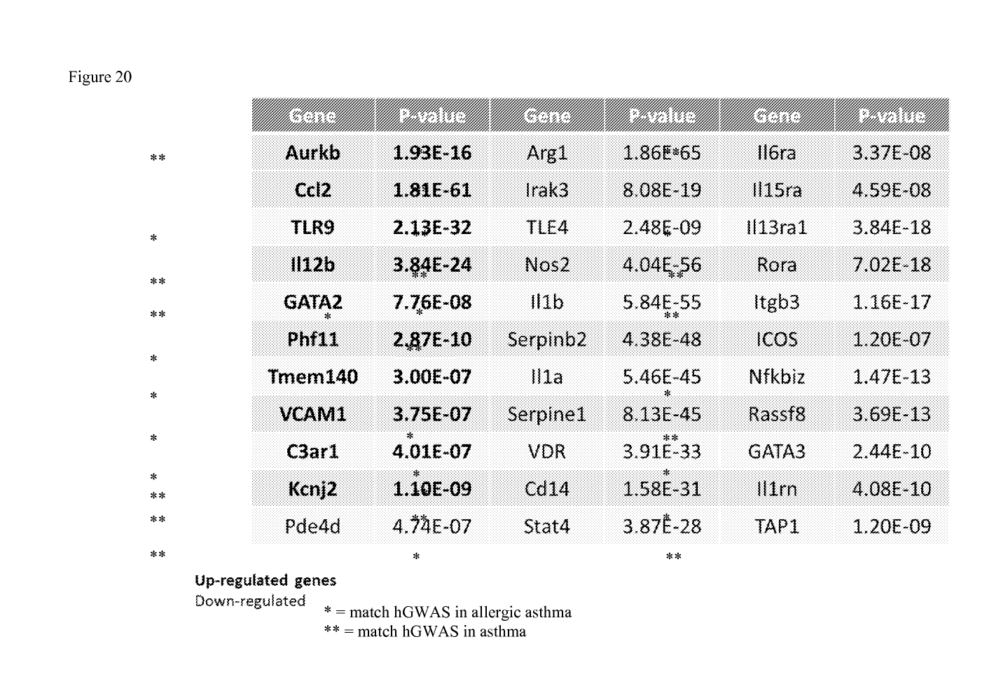

FIG. 21: cAMP levels and Gαs-Gαi signaling: Gαs-Gαi imbalanced signaling as a result of intracellular cAMP levels determines a pro-Th2 or pro-Th17 phenotype of dendritic cells where high intracellular cAMP levels lead to a pro-Th17 response and low intracellular cAMP levels lead to a pro-Th2 response, and treatment using the methods described herein can mediate the effects of the response and subsequent disease states by effecting the intracellular cAMP concentration.

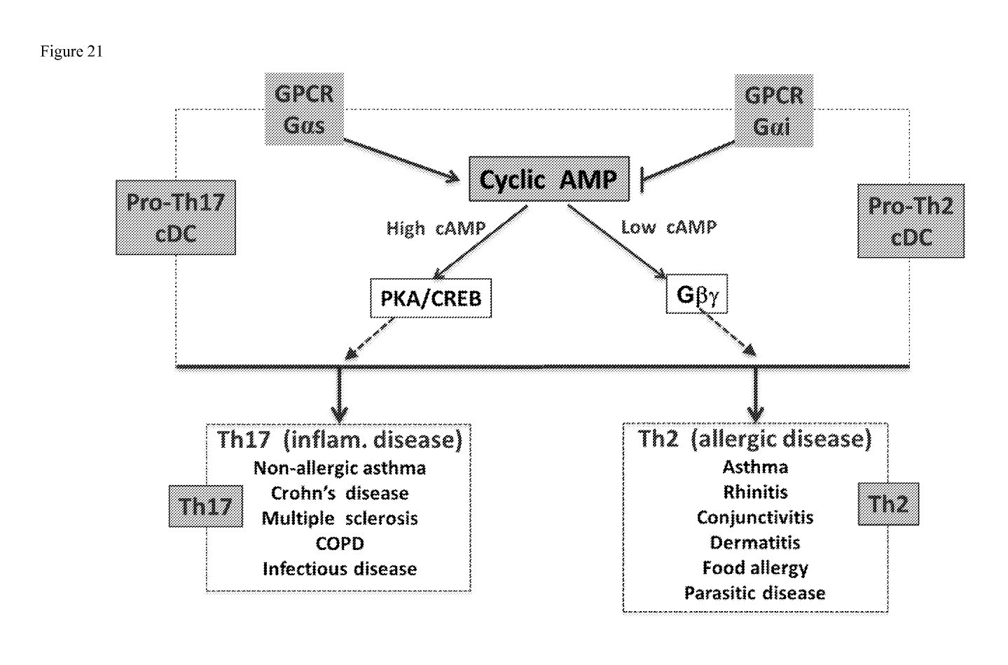

FIG. 22: Augmenting cAMP pathways in dendritic cells enhances Th1/Th17 responses: modulating dendritic cell intracellular cAMP levels using cAMP adjuvants that increase cAMP levels leads to inducement of Th cells into Th1/Th17 lineage which can stimulate immunity.

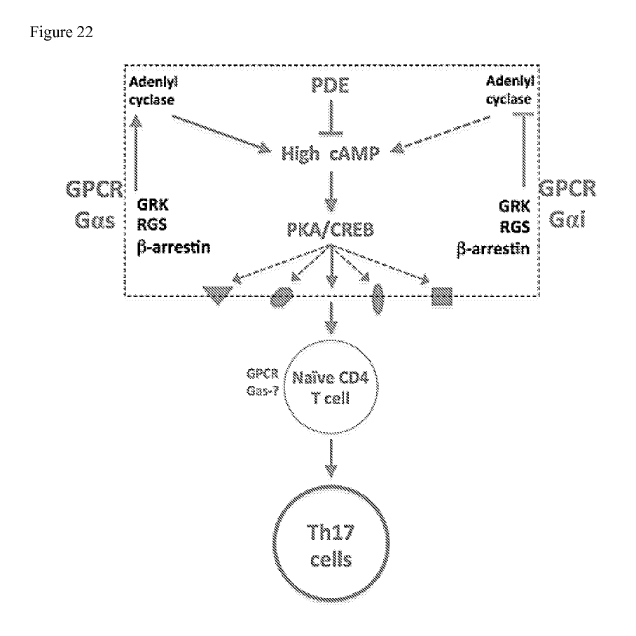

### DETAILED DESCRIPTION OF THE INVENTION

### I. Definitions

An “antigen presenting cell” or “APC” as used herein refers to an immune cell which displays antigens to T cells to mediate an immune response in an organism. An “activated-APC” refers to an APC having internal cAMP levels, which have been modulated with a cAMP-elevating agent or cAMP-lowering agent. Activated-APCs herein can induce selective differentiation of a subset of Th cells (e.g. Th1, Th2, Th17, or Treg cells). APCs include, for example, macrophages, basophils, dendritic cells and certain types of B-cells expressing B-cell receptor.

A “dendritic cell” or “DC” as used herein refers to an APC immune cell which processes and presents antigens to T cells to mediate an immune response in an organism. Dendritic cells instruct T helper (Th) cell differentiation. In embodiments, a dendritic cell may be a CD11c+ or CD11c− dendritic cell. In embodiments, a dendritic cell may be a blood dendritic cell (i.e. a dendritic cell isolated from a blood drawn sample).

The terms “Gαs” and “Gs” are herein used interchangeably and refer to G stimulatory alpha proteins. Gαs proteins are involved in increased intracellular cAMP via activation of adenylyl cyclase. The terms “Gαi” and “Gi” are herein used interchangeably and refer to G inhibitory alpha proteins. Gαi proteins are involved in decreased intracellular cAMP via deactivation of adenylyl cyclase and Gαs. The term “Gαs-Gαi pathway” refers to interactions between Gαs and/or Gαi with a GPCR and optionally other cellular components (e.g. proteins, nucleic acids, small molecules, ions, lipids) that convey a change in one component to one or more other components (e.g. activation of Gαi results in decreased cAMP production by deactivation of AC). In turn, this change may convey a change to additional components (e.g. further deactivation of Gαs), which is optionally propagated to other signaling pathway components (e.g. downstream regulation of GPCR post-signaling proteins such as GRK.).

An “agonist,” refers to a substance capable of detectably increasing the expression or activity of a given protein or compound. The agonist can increase expression or activity 10%, 20%, 30%, 40%, 50%, 60%, 70%, 80%, 90%, 100% or more in comparison to a control in the absence of the agonist. In embodiments, expression or activity is 1.5-fold, 2-fold, 3-fold, 4-fold, 5-fold, 10-fold or more higher than the expression or activity in the absence of the agonist. Thus, a Gαs-agonist is a compound that increases Gαs activity. Likewise, a PKA-agonist is a compound capable of increasing PKA activity. A CREB-agonist is a compound capable of increasing CREB activity. A Gαi-agonist increases Gαi activity or decreases Gαs activity. A GRK-agonist increases GRK activity. A RGS-agonist increases RGS activity. A b-arrestin-agonist increases b-arrestin activity. A PDE activator refers to a compound capable of increasing PDE activity.

The term “antagonist” refers to a substance capable of detectably lowering expression or activity of a given protein. The antagonist can inhibit expression or activity 10%, 20%, 30%, 40%, 50%, 60%, 70%, 80%, 90%, 100% or less in comparison to a control in the absence of the antagonist. In embodiments, the inhibition is 1.5-fold, 2-fold, 3-fold, 4-fold, 5-fold, 10-fold, or more than the expression or activity in the absence of the antagonist. Thus, a Gαi-antagonist decreases Gαi activity or increases Gαs activity. A GRK-antagonist decreases GRK activity. A RGS-antagonist decreases RGS activity. A b-arrestin-antagonist decreases b-arrestin activity. Likewise, a Gαs-antagonist decreases Gαs activity or increases Gαi activity. A PKA-antagonist decreases PKA activity. A CREB-antagonist decreases CREB activity. A PDE inhibitor refers to a compound capable of decreasing PDE activity.

The terms “differentiate,” “differentiation,” and “differentiating” are herein used interchangeably and refer to generation of a Th cell of a certain lineage (e.g., a Th2 cell) from a different type of cell (e.g., a naïve CD4+ cell). In embodiments, the phrases “lineage conversion” and “convert the lineage of” refers to changing the lineage of a cell that has already been set into a certain Th cell lineage and is considered “mature” (e.g. a Th17 cell) to a different Th cell lineage that is considered mature (e.g. a Th2 cell).

A “CD4 T cell” as used herein refers to a T cell, including but not limited to T helper (Th) cells, monocytes, macrophages, and dendritic cells which express the glycoprotein CD4. “A CD4+ naïve cell” refers to a CD4+ cell that has not yet been differentiated or been set in its lineage. A “mature-CD4 T cell” or “differentiated CD4 cell” refers to a CD4+ cell that has been differentiated, or otherwise set in its lineage into a Th cell (e.g. Th1, Th2, Th17 or Treg cell.

A “cAMP-elevating agent” refers to a compound (e.g. small molecule, peptide, antibody, nucleic acid, etc.) that increases the level or activity of cAMP in a cell. cAMP-elevating agents are well known in the art and include agents such as cAMP analogues, phosphodiesterase (PDE) inhibitors, Gαs-agonists (e.g. an agent capable of activating Gs or activating a GPCR that activates Gs), PKA-agonists, adenyl cyclase-agonists, CREB-agonists, Gαi-antagonists (e.g. an agent capable of inhibiting Gi or inhibiting a GPCR that activates Gi), GRK-antagonists, RGS-antagonists, or b-arrestin-antagonists. cAMP-elevating agents described herein may be bound to adjuvants, antigens, or allergens using conjugate chemistry as described herein.

An “adenyl cyclase-agonist” or “AC-agonist” is a compound that activates adenylate cyclase. Exemplary AC-agonists include forskolin (FK), cholera toxin (CT), pertussis toxin (PT) (e.g. an inhibitor of Gi), prostaglandins (e.g., PGE-1 and PGE-2), colforsin and P-adrenergic receptor agonists, such as albuterol, bambuterol, bitolterol, carbuterol, clenbuterol, clorprenaline, denopamine, dioxethedrine, dopexamine, ephedrine, epinephrine, etafedrine, ethylnorepinephrine, fenoterol, formoterol, hexoprenaline, ibopamine, isoetharine, isoproterenol, mabuterol, metaproterenol, methoxyphenamine, norepinephrine, oxyfedrine, pirbuterol, prenalterol, procaterol, propranolol, protokylol, quinterenol, reproterol, rimiterol, ritodrine, salmefamol, soterenol, salmeterol, terbutaline, tretoquinol, tulobuterol, and xamoterol.

A “phosphodiesterase-inhibitor” or “PDE-inhibitor” is a compound that inhibits a cAMP phosphodiesterase. Exemplary PDE-inhibitors include amrinone, milrinone, xanthine, methylxanthine, anagrelide, cilostamide, medorinone indolidan, rolipram, 3-isobutyl-1-methylxanthine (IBMX), chelerythrine, cilostazol, glucocorticoids, griseolic acid, etazolate, caffeine, indomethacin, papverine, MDL 12330A, SQ 22536, GDPssS, clonidine, type III and type IV phosphodiesterase inhibitors, methylxanthines such as pentoxifylline, theophylline, theobromine, pyrrolidinones and phenyl cycloalkane and 5 cycloalkene derivatives, lisophylline, and fenoxammne.

A “cAMP analogue” is a compound capable of mimicking the function of cAMP in an intracellular environment and which is structurally related to cAMP. Exemplary cAMP analogues include dibutyrylcAMP (db-cAMP), (8-(4)-chlorophenylthio)-cAMP (cpt-cAMP), 8-[(4-bromo-2,3-dioxo buty 1)thio]-cAMP, 2-[(4-bromo-2,3-dioxo butyl)thio]-cAMP, 8-bromo-cAMP, dioctanoy 1-cAMP, Sp-adenosine 3′:5′-cyclic phosphorothioate, 8-piperidino-cAMP, N.sup.6-phenyl-cAMP, 8-methylamino-cAMP, 8-(6-aminohexyl)amino-cAMP, 2′-deoxy-cAMP, N.sup.6,2′-0-dibutryl-1 0 cAMP, N.sup.6,2′-0-disuccinyl-cAMP, N.sup.6-monobutyryl-cAMP, 2′-0-monobutyryl-cAMP, 2′-0-monobutryl-8-bromo-cAMP, N.sup.6-monobutryl-2′-deoxy-cAMP, and 2′-0-monosuccinyl-cAMP. Additional cAMP analogues are also known in the art.

A “cAMP-lowering agent” refers to a compound (e.g. small molecule, peptide, antibody, nucleic acid, etc.) that decreases the level or activity of cAMP in a cell. cAMP-lowering agents are well known in the art and include agents such as Gαs-antagonists (e.g. an agent capable of inhibiting Gs or inhibiting a GPCR that activates Gs), PKA-antagonists, adenyl cyclase-antagonists, CREB-antagonists, PDE activators, Gαi-agonists (e.g. an agent capable of activating Gi or activating a GPCR that activates Gi), GRK-agonists, RGS-agonists, or b-arrestin-agonists. cAMP-lowering agents described herein may be bound to adjuvants, antigens, or allergens using conjugate chemistry as described herein.

cAMP-elevating agents and cAMP-lowering agents can be administered to a subject (e.g. a mammalian subject such as a human subject) for the treatment of any of the diseases or conditions described herein. As described in detail herein, the cAMP-elevating agents and cAMP-lowering agents are administered in any suitable manner, optionally with pharmaceutically acceptable carriers.

cAMP-elevating agents and cAMP-lowering agents described herein, including embodiments thereof, may be formulated with a pharmaceutically acceptable carrier. cAMP-elevating agents and cAMP-lowering agents described herein, including embodiments thereof, may be bound to a pharmaceutically acceptable carrier. The pharmaceutically acceptable carrier is as described herein.

“Conjugate chemistry” as described herein includes coupling two molecules together to form an adduct. Conjugation may be a covalent modification. Currently favored classes of conjugate chemistry reactions available with reactive known reactive groups are those that proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups used for conjugate chemistries herein include, for example: carboxyl groups; hydroxyl groups, haloalkyl groups; dienophile groups; aldehyde or ketone; sulfonyl halide groups; thiol groups, amine or sulfhydryl groups; alkenes; epoxides; phosphoramidites; metal silicon oxide bonding; metal bonding to reactive phosphorus groups (e.g. phosphines) and azides coupled to alkynes using copper catalyzed cycloaddition click chemistry.

The reactive functional groups can be chosen such that they do not participate in, or interfere with, the chemical stability of the conjugate described herein. Alternatively, a reactive functional group can be protected from participating in the crosslinking reaction by the presence of a protecting group. In embodiments, a cAMP-elevating agent or cAMP-lowering agent as described herein is conjugated to an antigen, allergen, or adjuvant as described hereinabove.

“Pharmaceutically acceptable excipient,” “pharmaceutically acceptable carrier,” or “carrier” refers to pharmaceutical excipients, for example, pharmaceutically, physiologically, acceptable organic or inorganic carrier substances suitable for enteral or parenteral application that do not deleteriously react with the active agent. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration. Preparations may include nanoparticles.

A “test compound” as used herein refers to an experimental compound used in a screening process to identify activity, non-activity, or other modulation of a particularized biological target or pathway.

As defined herein, the term “activation”, “activate”, “activating” and conjugations thereof in reference to a protein refers to conversion of a protein into a biologically active derivative from an initial inactive or deactivated state. The terms reference activation, or activating, sensitizing, or up-regulating signal transduction or enzymatic activity or the amount of a protein in a disease.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments, inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation).

The term “cAMP modulator” refers to a composition that increases or decreases the level of intracellular cAMP or cAMP function in a cell (e.g. an antigen presenting cell). The term “modulate” is used in accordance with its plain ordinary meaning and refers to the act of changing or varying one or more properties. “Modulation” refers to the process of changing or varying one or more properties. For example, as applied to the effects of a modulator on cAMP levels, to modulate means to change by increasing or decreasing the level of cAMP internally in an antigen presenting cell.

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound (e.g. cAMP).

The term “expression” includes any step involved in the production of a polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc).

The terms “disease” or “condition” refer to a state of being or health status of a patient or subject capable of being treated with the compounds or methods provided herein.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life or engraftment potential) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant. In embodiments, the control is used as a standard of comparison in evaluating experimental effects. In embodiments, a control is the measurement of the activity of a protein in the absence of a compound as described herein (including embodiments and examples).

The terms “treating” or “treatment” refer to any indicia of success in the treatment or amelioration of an injury, disease, pathology or condition, including any objective or subjective parameter such as abatement; remission; diminishing of symptoms or making the injury, pathology or condition more tolerable to the patient; or slowing in the rate of progression of a disease. As used herein, the terms “treat” and “prevent” are not intended to be absolute terms. Treatment can refer to any delay in onset, amelioration of symptoms, decreased inflammation, decreased Th2-response or decreased Th17-response. The effect of treatment can be compared to an individual or pool of individuals not receiving the treatment, or to the same patient prior to treatment or at a different time during treatment. The treatment or amelioration of symptoms can be based on objective or subjective parameters; including the results of a physical examination, neuropsychiatric exams, and/or a psychiatric evaluation. The terms “prevent” or “prevention” and conjugations thereof refer to any indicia of success in the amelioration of a disease, pathology or condition. As used herein, the term and “prevent” is not intended to be absolute terms. Prevention can refer to any delay in onset, amelioration of symptoms, decreased inflammation, decreased Th2-response or decreased Th17-response. Prevention may refer to preventing the onset of a disease through vaccination.

The terms “phenotype” and “phenotypic” as used herein refer to an organisms observable characteristics such as onset or progression of disease symptoms, biochemical properties, or physiological properties.

“Contacting” is used in accordance with its plain ordinary meaning and refers to the process of allowing at least two distinct species (e.g. agent (e.g. activator, inhibitor), chemical compounds including biomolecules, or cells) to become sufficiently proximal to react, interact or physically touch. It should be appreciated; however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be an agonist or antagonist as described herein and a protein. In some embodiments, contacting includes allowing an agonist or antagonist described herein to interact with a protein that is involved in a signaling pathway.

“Patient,” “patient in need thereof,” or “subject in need thereof” refers to a living organism suffering from or prone to a disease or condition that can be treated by administration of agonists or antagonists provided herein. Non-limiting examples include humans, other mammals, bovines, rats, mice, dogs, monkeys, goat, sheep, cows, deer, and other non-mammalian animals. In embodiments, a patient is human. The term does not necessarily indicate that the subject has been diagnosed with a particular disease, but typically refers to an individual under medical supervision.

The term “sample” includes sections of tissues such as biopsy and autopsy samples, and frozen sections taken for histological purposes. Such samples include blood and blood fractions or products (e.g., serum, plasma, platelets, white or red blood cells, and the like), sputum, tissue, cultured cells (e.g., primary cultures, explants, and transformed cells), stool, urine, other biological fluids (e.g., prostatic fluid, gastric fluid, intestinal fluid, renal fluid, lung fluid, cerebrospinal fluid, and the like), etc. A sample is typically obtained from a “subject” such as a eukaryotic organism, most preferably a mammal such as a primate, e.g., chimpanzee or human; cow; dog; cat; a rodent, e.g., guinea pig, rat, mouse; rabbit; or a bird; reptile; or fish. In embodiments, the sample is obtained from a human.

An “effective amount” or “therapeutically effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of a disease, pathology or condition, or reducing the likelihood of the onset (or reoccurrence) of a disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays or using the Gαs knockout mouse described herein. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, intraperitoneal, intramuscular, intralesional, intrathecal, inhalation or intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies. The compounds of the invention can be administered alone or can be coadministered to the patient. Coadministration is meant to include simultaneous or sequential administration of the compounds individually or in combination (more than one composition). Thus, the preparations can also be combined, when desired, with other active substances (e.g. to reduce metabolic degradation). The compositions of the present invention can be delivered transdermally, by a topical route, or formulated as applicator sticks, solutions, suspensions, emulsions, gels, creams, ointments, pastes, jellies, paints, powders, and aerosols.

A “Th2-mediated disease” refers to a disease caused by induction of a Th2 cell response. A Th2-mediated disease may be caused by Th2 cell production in response to the presence of an allergen, antigen, or parasitic infection. A chronic Th2-mediated disease is a disease which has ongoing symptoms for an extended period of time (e.g. at least 1 year). As used herein, a Th2-mediated disease may refer to a Th2-response originating from lowered intracellular cAMP levels in a dendritic cell in response to changes in a Gαs/Gαi pathway. Exemplary Th2-mediated diseases include allergic asthma, rhinitis, conjunctivitis, colitis, dermatitis, food allergies, insect venom allergies, and anaphylaxis.

A “Th2 response” may refer to production of Th2 cells in response to a condition. In embodiments, the Th2 response results in the symptoms of the disease (e.g. allergic asthma). In embodiments, the Th2 response is in response to the presence of an infection. In embodiments, the infection may be a helminth or parasite infections. In such embodiments, the Th2 response mitigates the parasitic or helminth infection.

A “Th17-mediated disease” refers to a disease caused by induction of a Th17 cell response. In embodiments, the Th17 response results in symptoms of the disease (e.g. inflammation). As used herein, a Th17-mediated disease typically refers to a Th17-response originating from increased intracellular cAMP levels in a dendritic cell in response to changes in a Gαs/Gαi pathway. Exemplary Th17-mediated diseases include non-allergic asthma, Crohn's Disease, multiple sclerosis, and COPD.

A “Th17 response” may refer to production of Th17 cells in response to a condition. In embodiments, the Th17 response results in the symptoms of the disease (e.g. Multiple Sclerosis, or Crohn's Disease). In embodiments, the Th17 response is in response to the presence of an infection, wherein the increased presence of Th17 cells mitigates the infection.

An “adjuvant” as used herein refers to an agent that increases the effect of a cAMP-elevating agent or a cAMP-lowering agent as set forth herein. In embodiments, the adjuvant increases cell delivery of the cAMP-elevating agent or cAMP-lowering agent. Thus, in embodiments, the adjuvant is a cell-delivery agent. Exemplary cell-delivery agents include oil emulsions, liposomes, nanoparticles, complementary-adjuvant combinations (e.g. adjuvants absorbed to or bound (e.g. chemical conjugation of an antigen to a cAMP-elevating agent or to a cAMP-lowering agent) to another adjuvant (e.g. alum)). In embodiments, the adjuvant system includes a cAMP-elevating agent absorbed to alum. In embodiments, an adjuvant system included a cAMP-lowering agent absorbed to alum. In embodiments, adjuvants and adjuvant systems described herein are used in vaccination to provoke a protective immune response. In embodiments, the adjuvant is a pharmacological or immunological agent that enhances antigen immunogenicity (i.e. enhance an immune response) and/or modulates the type of protective immunity (e.g., humoral vs. cellular immune response). Thus, in embodiments, the adjuvant is an immunostimulating-agent. In embodiments, the immunostimulating-agent optionally activates the two arms of the immune system (e.g. innate immunity (preferably dendritic cells) and adaptive immunity, including CD4 T cells, CD8 T cells and B cells). In embodiments, the adjuvant stimulates expression of GPCRs. Thus, in embodiments, the adjuvant is a GPCR-stimulating agent. Exemplary adjuvants include alum, TLR9-agonists, TLR9 ligands, TLR2 ligands, MF59, or TLR4-agonists.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N.Y. 1989). Any methods, devices and materials similar or equivalent to those described herein can be used in the practice of this invention. The following definitions are provided to facilitate understanding of certain terms used frequently herein and are not meant to limit the scope of the present disclosure.

### II. Methods of Inducing CD4 T Cell Differentation

In a first aspect is a method of inhibiting dendritic cell induction of CD4 T cell lineage conversion to a Th2 cell. The method includes contacting a dendritic cell with a cAMP-elevating agent in the presence of a CD4 T cell. The cAMP concentration within the dendritic cell is allowed to increase relative to the absence of the cAMP-elevating agent thereby inhibiting dendritic cell induction of lineage conversion of the CD4 T cell to a Th2 cell. The cAMP-elevating agent is exogenous to the dendritic cell.

The CD4 T cell may be a naïve CD4 T cell or a mature CD4 cell (e.g. Th1, Th2, Th17, or Treg cell). The CD4 T cell may be a naïve CD4 T cell. The CD4 T cell may be a Th1 cell. The CD4 T cell may be a Th2 cell. The CD4 T cell may be a Th17 cell. The CD4 T cell may be a Treg cell. The CD4 T cell or the dendritic cell may form part of an organism. The organism may be a mammal, including, for example, a human. The cAMP concentration within the dendritic cell may be compared to a control.

The cAMP-elevating agent is an agent as described herein that is capable of increasing the cAMP concentration within an antigen presenting cell (“APC”). In embodiments, the cAMP-elevating agent is a Gαs-agonist, a PKA-agonist, a CREB-agonist, a cAMP analogue, a PDE inhibitor, a Gαi-antagonist, a GRK-antagonist, a RGS-antagonist, or a b-arrestin-antagonist. The cAMP-elevating agent may be a Gαs-agonist (e.g. PGE2). The cAMP-elevating agent may be a PKA-agonist. PKA-agonists are well known in the art and can include, for example, N6. The cAMP-elevating agent may be an AC-agonist. AC-agonists are well known in the art and include, for example, forskolin, CT or PT. The cAMP-elevating agent may be a CREB-agonist. The cAMP-elevating agent may be a cAMP analogue. The cAMP analogue is described herein, including embodiments thereof. The cAMP analogue may be a PDE inhibitor (e.g. IBMX). The cAMP-elevating agent may be a Gαi-antagonist. The cAMP-elevating agent may be a GRK-antagonist. The cAMP-elevating agent may be a RGS-antagonist. The cAMP-elevating agent may be a b-arrestin-antagonist. The cAMP-elevating agent may be absorbed to an adjuvant. In embodiments, the cAMP-elevating agent may be covalently bound (e.g. using conjugate chemistry) to an adjuvant. The adjuvant may be alum. In embodiments, the method includes the addition of an antigen. The antigen may be covalently bound (e.g. using conjugate chemistry) to the cAMP-elevating agent.

In another aspect is a method of activating dendritic cell induction of CD4 T cell lineage conversion to a Th2 cell. The method includes contacting a dendritic cell with a cAMP-lowering agent in the presence of a CD4 T cell. The cAMP concentration within the dendritic cell is allowed to decrease relative to the absence of the cAMP-lowering agent thereby activating dendritic cell induction of lineage conversion of the CD4 T cell to a Th2 cell. The CD4 T cell and dendritic cell are as described herein, including embodiments thereof. The cAMP concentration may be compared to a control.

The cAMP-lowering agent is an agent capable of lowering cAMP levels in an APC. In embodiments, the cAMP lowering agent is a Gαs-antagonist, a PKA-antagonist, a CREB-antagonist, a PDE activator, a Gαi-agonist, a GRK-agonist, a RGS-agonist, or a b-arrestin-agonist. The cAMP-lowering agent may be a Gαs-antagonist. The cAMP-lowering agent may be a PKA-antagonist. PKA-antagonists are well known in the art and include, for example, H-89. The cAMP-lowering agent may be a CREB-antagonist. The cAMP-lowering agent may be a PDE activator. The cAMP-lowering agent may be a Gαi-agonist. The Gαi-agonist may stimulate Gαi and further antagonize Gαs through a feedback mechanism. In certain embodiments, the Gαi and Gαs activities depend on the relative expression of each (i.e. higher Gαi expression further inhibits Gαs and higher Gαs expression further inhibits Gαi). The cAMP-lowering agent may be a GRK-agonist. The cAMP-lowering agent may be a RGS-agonist. The cAMP-lowering agent may be a b-arrestin-agonist. The cAMP-lowering agent may be absorbed to an adjuvant. The adjuvant may be alum.

In another aspect is a method of inhibiting dendritic cell induction of CD4 T cell lineage conversion to a Th17 cell. The method includes contacting a dendritic cell with a cAMP-lowering agent in the presence of a mature CD4 T cell. The cAMP concentration within the dendritic cell is allowed to decrease relative to the absence of the cAMP-elevating agent thereby inhibiting dendritic cell induction of lineage conversion of the mature CD4 T cell to a Th17 cell. The cAMP-lowering agent is exogenous to the dendritic cell. The mature CD4 T cell may be a Th1 cell. The mature CD4 T cell may be a Th2 cell. The mature CD4 T cell may be a Th17 cell. The mature CD4 T cell may be a Treg cell. The mature CD4 T cell or the dendritic cell may form part of an organism. In embodiments, the first mature CD4 T cell is a CD4 T cell whose lineage is set (e.g. a Th17 cell) and is allowed to convert to a different lineage thereby resulting in a different (e.g. second) CD4 T cell. The mechanism of conversion may result in a change in the expression of a cytokines or proteins (e.g. IL-4, IL-5, IL-6, IL-10, IL-13, INFγ, or TGFβ) from the first mature CD4 T cell to those expressed by the second CD4 T cell. The organism may be a mammal, including, for example, a human. The cAMP concentration within the dendritic cell may be compare to a control. The cAMP-lowering agent is an agent as described herein, including embodiments thereof.

In another aspect is a method of activating dendritic cell lineage conversion of CD4 T cell to a Th17 cell. The method includes contacting a dendritic cell with a cAMP-elevating agent in the presence of a mature CD4 T cell. The cAMP concentration within the dendritic cell is allowed to increase relative to the absence of the cAMP-elevating agent thereby activating dendritic cell induction of lineage conversion of the mature CD4 T cell to a Th17 cell. The mature CD4 T cell and the dendritic cell are as described herein, including embodiments thereof. The cAMP concentration may be compared to a control. The cAMP-elevating agent is as described herein, including embodiments thereof.

In another aspect is a method of inducing mature CD4 T cell lineage conversion using an APC. The method includes contacting an APC with a cAMP-lowering agent. The cAMP-lowering agent is allowed to lower cAMP levels in the APC, thereby forming an activated-APC. The activated-APC is contacted with a first mature CD4 T cell. The activated-APC is allowed to convert the lineage of the first mature CD4 T cell to a second mature CD4 T cell, thereby inducing CD4 T cell lineage conversion using an APC. The APC may be a dendritic cell or a macrophage, as described herein, including embodiments thereof. The APC may be part of an organism, such as a mammal. The organism may be a human. The cAMP-lowering agent is an agent described herein, including embodiments thereof.

The first mature CD4 T cell may be a cell from a CD4 Th subset (e.g. Th1, Th2, Th17 or Treg). The lineage of the first mature CD4 T cell may be converted to a cell from a CD4 Th subset (e.g. Th1, Th2, Th17, or Treg). In embodiments, a Th1 cell is converted to a Th2 cell using the methods herein. The Th1 cell may be part of an organism, such as, for example a human. In embodiments, a Th17 cell is converted to a Th2 cell using the methods herein. The Th17 cell may be part of an organism, such as, for example a human.

In another aspect is a method of inducing mature CD4 T cell lineage conversion using an APC. The method includes contacting an APC with a cAMP-elevating agent. The cAMP-elevating agent is allowed to increase cAMP levels in the APC, thereby forming an activated-APC. The activated-APC is contacted with a first mature CD4 T cell. The activated-APC is allowed to convert the lineage of the first mature CD4 T cell into a second mature CD4 T cell, thereby inducing CD4 T cell lineage conversion using an APC. The APC is as described herein, including embodiments thereof. The cAMP-elevating agent is an agent described herein, including embodiments thereof.

In embodiments, a Th1 cell is converted to a Th17 cell using the methods herein. The Th17 cell may be part of an organism, such as, for example a human. In embodiments, a Th2 cell is converted to a Th17 cell using the methods herein. The Th2 cell may be part of an organism, such as, for example a human.

### III. Methods for Treating Th2-Mediated Diseases or Th17-Mediated Diseases

In another aspect is a method of treating a Th2-mediated disease in a patient in need thereof. The method includes administering to the patient an effective amount of a cAMP-elevating agent. The cAMP-elevating agent may increase the intracellular levels of cAMP in an APC. In embodiments, treating a Th2-mediated disease is performed by decreasing the Th2-response or decreasing the number of Th2 cells. Described herein are methods to decrease a Th2-response or decrease the number of Th2 cells by inhibiting dendritic cell induction of CD4 T cells (e.g. naïve or mature T cells) to Th2 cells. The decreased response or cell number is attained through modulation of the Gαs/Gαi pathways as described herein. Thus, when a dendritic cell exhibits elevated intracellular cAMP levels, it will inhibit lineage conversion of CD4 T cells (e.g. naïve or mature T cells) to Th2 cells. The method may further include administering to the patient an adjuvant in combination with the cAMP-elevating agent (i.e. co-administration). The adjuvant may be alum.

The triggering of the elevated cAMP levels in the APC may form an activated-APC capable of converting the lineage of a naïve CD4 cell to a Th cell subclass such as Th1 or Th17, thereby reducing the expression levels of Th2 cells. The triggering of the elevated cAMP levels in the APC may form an activated-APC capable of converting the lineage of a Th2 cell into a different Th cell subclass, such as, for example, Th1 or Th17. The conversion may minimize the Th2 cell count thereby alleviating the aggravating expression of Th2 cells causing the symptoms of the disease.

The cAMP-elevating agent is as described herein, including embodiments thereof. The treated Th2-mediated disease may be allergic asthma, rhinitis, conjunctivitis, dermatitis, colitis, food allergy, insect venom allergy, drug allergy or anaphylaxis-prone conditions. The treated Th2-mediated disease may be allergic asthma, which may be characterized by the presence of hypersensitivity and inflammation of bronchial airways in response to an allergen. The treated Th2-mediated disease may be allergic rhinitis, which may be characterized by the presence of inflammation of the nasal airways in response to an allergen. The treated Th2-mediated disease may be allergic conjunctivitis, which may be characterized by the presence of inflammation of the conjunctiva in response to an allergen. The treated Th2-mediated disease may be allergic dermatitis, which may be characterized by hypersensitivity of the skin in response to contact with an allergen. The treated Th2-mediated disease may be a drug allergy. The treated Th2-mediated disease may be colitis, which may be characterized by colitogenic Th2 cells within the colon. The treated Th2-mediated disease may be a food allergy. One skilled in the art would readily recognize many types of food allergies exist and that such responses are due to immunological allergic responses. Thus one skilled in the art would recognize that food allergies to such types of food as corn, egg, fish, meat, milk, peanut, shellfish, soy, tree nuts, or wheat, are non-limiting examples. Likewise, one skilled in the art would readily recognize many insect venom allergies exist and that such responses are due to immunological allergic responses. Thus, one skilled in the art would recognize that insect venom allergies to such types of bites or stings from bees (e.g. wasps, yellowjackets, and hornets), ants, mosquitoes and ticks are non-limiting examples.

In another aspect is a method for treating a Th2-mediated disease in a patient in need thereof. The method includes administering to the patient an effective amount of a cAMP-lowering agent.

In embodiments, treating a Th2-mediated disease is performed by increasing the Th2-response or the number of Th2 cells. Described herein are methods to increase a Th2-response or increase the number of Th2 cells by activating dendritic cell lineage conversion of CD4 T cells (e.g. naïve or mature T cells) to Th2 cells. The increased response or number is attained through modulation of the Gαs/Gαi pathways as described herein. Thus, the triggering of the lowered cAMP levels in the APC may form an activated-APC capable of converting the lineage of a naïve CD4 T cell to a Th2 cell. The triggering of the lowered cAMP levels in the APC may form an activated-APC capable of converting the lineage of a mature T cell other Th cell subclasses, such as, for example, Th1 or Th17 into a Th2. The increased Th2-response is useful for treating parasitic infections and helminthic infections. The cAMP-lowering agent is as described herein, including embodiments thereof. The Th2-mediated diseases are as described herein, including embodiments thereof. The method may further include administering to the patient an adjuvant in combination with the cAMP-lowering agent (i.e. co-administration). The adjuvant may be alum. In embodiments, the cAMP-lowering agent may be absorbed to the adjuvant. In embodiments, the cAMP-lowering agent may be covalently bound (e.g. using conjugate chemistry) the adjuvant. In embodiments, the method includes the addition of an antigen. The antigen may be covalently bound (e.g. using conjugate chemistry) to the cAMP-lowering agent.

In another aspect is a method of treating a Th2-mediated disease by inhibiting gene targets identified by a micro array and comparing gene expression in wild type dendritic cells to that in Gαs-knockout dendritic cell that regulate Th2 differentiation. The gene targets may be genes that express proteins in the Gαs/Gαi pathway. The Th2-mediated disease is as described herein.

In another aspect is a method of treating a Th2-mediated disease by adoptive transfer of dendritic cells. The dendritic cells may be loaded in vitro with a cAMP-elevating agent or a cAMP-lowering agent to form a loaded-dendritic cell. The dendritic cell may include an allergen or an antigen. The allergen is an allergen that stimulates a Th2-response (e.g. a food that provokes a food allergy). The antigen is an antigen that stimulates a Th2-response (e.g. a helminth infection that provokes Th2 cell production). In embodiments, the cAMP elevating agent or cAMP-lowering agent is bound to the antigen. The cAMP-elevating agent or cAMP-lowering agent may be conjugated to the antigen using conjugation chemistry as described herein, including embodiments thereof. In embodiments, the cAMP elevating agent or cAMP-lowering agent is bound to the allergen. The cAMP-elevating agent or cAMP-lowering agent may be conjugated to the allergen using conjugation chemistry as described herein, including embodiments thereof. The loaded-dendritic cell may be administered to a patient in need thereof. The cAMP-elevating agent or cAMP-lowering agent is as described herein, including embodiments thereof. The dendritic cell is as described herein, including embodiments thereof. The Th2-mediated disease is as described herein.

In another aspect is a method of treating a Th17-mediated disease in a patient in need thereof. The method includes administering to the patient an effective amount of a cAMP-lowering agent. The cAMP-lowering agent may decrease the intracellular levels of cAMP in an APC, thereby promoting lineage conversion of a Th17 cell to a mature CD4 cell. In embodiments, treating a Th17-mediated disease is performed by decreasing the Th17-response or decreasing the number of Th17 cells. Described herein are methods to decrease a Th17-response or decrease the number of Th17 cells by inhibiting dendritic cell lineage conversion of CD4 T cells (naïve or mature T cells) to Th17 cells. The decreased response or cell number may be attained through modulation of the Gαs/Gαi pathways as described herein. In embodiments, the decreased response results from modulation the Gαs/Gαi pathways in favor of Gαi. Thus, when a dendritic cell exhibits lowered intracellular cAMP levels, it may inhibit lineage conversion of naïve CD4 T cells to Th17 cells. When a dendritic cell exhibits lowered intracellular cAMP levels, it may inhibit lineage conversion of mature CD4 T cells to Th17 cells. When a dendritic cell exhibits lowered intracellular cAMP levels, it may promote lineage conversion of Th17 cells to mature a CD4 T cell, such as a Th2 cell. The cAMP-lowering agent is as described herein, including embodiments thereof. The treated Th17-mediated disease is Th17 mediated diseases described herein. The mature CD4 cell may be a Th1 or Th2 cell. The method may further include administering to the patient an adjuvant in combination with the cAMP-lowering agent (i.e. co-administration). The adjuvant may be alum. In embodiments, the cAMP-lowering agent may be absorbed to the adjuvant. In embodiments, the cAMP-lowering agent may be covalently bound (e.g. using conjugate chemistry) the adjuvant. In embodiments, the method includes the addition of an antigen. The antigen may be covalently bound (e.g. using conjugate chemistry) to the cAMP-lowering agent.

In another aspect is a method for treating a Th17-mediated disease in a patient in need thereof. The method includes administering to the patient an effective amount of a cAMP-elevating agent. In embodiments, treating a Th17-mediated disease is performed by increasing the Th17-response or the number of Th17 cells. Described herein are methods to increase a Th17-response or increase the number of Th17 cells by activating dendritic cell induction of lineage conversion of CD4 T cells to Th17 cells. The increased response or number is attained through modulation of the Gαs/Gαi pathways as described herein. In embodiments, the decreased response results from modulation the Gαs/Gαi pathways in favor of Gαs. Thus, the triggering of the elevated cAMP levels in the APC may form an activated-APC capable of converting the lineage of a naïve CD4 T cell to a Th17 cell. When a dendritic cell exhibits elevated intracellular cAMP levels, it may promote lineage conversion of mature CD4 T cells to Th17 cells. The cAMP-elevating agent is as described herein, including embodiments thereof. The Th17-mediated diseases are as described herein. The method may further include administering to the patient an adjuvant in combination with the cAMP-elevating agent (i.e. co-administration). The adjuvant may be alum.

In another aspect is a method of treating a Th17-mediated disease by inhibiting gene targets identified by a micro array and comparing gene expression in wild type dendritic cells to that in Gαs-knockout dendritic cell that regulate Th17 differentiation. The gene targets may be genes that express proteins in the Gαs/Gαi pathway. The Th17-mediated disease is as described herein.

In another aspect is a method of treating a Th17-mediated disease by adoptive transfer of dendritic cells. The dendritic cells may be loaded in vitro with a cAMP-lowering agent to form a loaded-dendritic cell. In embodiments, the cAMP-lowering agent may be absorbed to an adjuvant. In embodiments, the cAMP-lowering agent may be covalently bound (e.g. using conjugate chemistry) an adjuvant. In embodiments, the method includes the addition of an antigen. The antigen may be covalently bound (e.g. using conjugate chemistry) to the cAMP-lowering agent. The loaded-dendritic cell may be administered to a patient in need thereof. The cAMP-lowering agent is as described herein, including embodiments thereof. The dendritic cell is as described herein, including embodiments thereof. The Th17-mediated disease is as described herein.

In another aspect is a method of treating a Th2-mediated disease in a patient in need thereof. The method includes detecting a cAMP level in a patient sample. The cAMP level is compared to a control thereby identifying a low cAMP level in the patient sample. An effective amount of a cAMP-elevating agent is then administered to the patient thereby treating the Th2-mediated disease. The cAMP-elevating agent is as described herein, including embodiments thereof. The Th2-mediated disease is as described herein, including embodiments thereof. The Th2-mediated disease also includes induction of a Th2-response for treating parasitic and helminthic infections as described herein, including embodiments thereof. The patient sample may be a biopsy or a blood draw. The patient sample may contain APCs, including dendritic cells. The patient sample may contain peripheral blood mononuclear cells (PBMC). In embodiments, the detection occurs after activation of a Gαs or Gαi pathway using an agonist or antagonist as described herein.

In another aspect is a method of identifying a Th2-mediated disease in a patient. The method includes detecting a cAMP level in a patient sample. The cAMP level is compared to a control to identify a low cAMP level in the patient sample, thereby identifying a Th2-mediated disease. The cAMP-elevating agent is as described herein, including embodiments thereof. The Th2-mediated disease is as described herein, including embodiments thereof. The patient sample may be a biopsy or a blood draw. The patient sample may contain APCs, including dendritic cells. The patient sample may contain peripheral blood mononuclear cells (PBMC). In embodiments, the detection occurs after activation of a Gαs or Gαi pathway using an agonist or antagonist as described herein.

In another aspect is a method of treating a Th17-mediated disease in a patient in need thereof. The method includes detecting a cAMP level in a patient sample. The cAMP level is compared to a control thereby identifying a high cAMP level in the patient sample. An effective amount of a cAMP-lowering agent is then administered to the patient thereby treating the Th17-mediated disease. The cAMP-lowering agent is as described herein, including embodiments thereof. The Th17-mediated disease is as described herein, including embodiments thereof. The patient sample may be a biopsy or a blood draw. The patient sample may contain APCs, including dendritic cells. The patient sample may contain peripheral blood mononuclear cells (PBMC). In embodiments, the detection occurs after activation of a Gαs or Gαi pathway using an agonist or antagonist as described herein.

In another aspect is a method of identifying a Th17-mediated disease. The method includes detecting a cAMP level in a patient sample. The cAMP level is compared to a control to identify a high cAMP level in the patient sample, thereby identifying a Th17-mediated disease. The cAMP-lowering agent may activate an APC to induce lineage conversion of a Th17 cell to a mature CD4 T cell (e.g. Th1 or Th2). The cAMP-lowering agent may be a Th17-cell lineage conversion agent (e.g. an agent that converts the lineages of a Th17 cell to a mature CD4 T cell). In embodiments, the lowered expression of Th17 cells mediates the Th17-response and treats a Th17-mediated disease. The cAMP-lowering agent is as described herein, including embodiments thereof. The Th17-mediated disease is as described herein, including embodiments thereof. The mature CD4 T cell is as described herein, including embodiments thereof. The patient sample may be a biopsy or a blood draw. The patient sample may contain APCs. The patient sample may contain PBMCs. In embodiments, the detection occurs after activation of a Gαs or Gαi pathway using an agonist or antagonist as described herein.

In another aspect is a method of distinguishing between a Th2-mediated disease and Th17-mediated disease in a patient. The symptoms of the Th2-mediated disease are similar (e.g. identical) to the Th17-mediated disease. The method includes taking a patent sample and detecting a cAMP level in the patient sample. The cAMP level is compared to a control to identify the cAMP level in the patient sample. A low cAMP level indicates a Th2-mediated disease. A high cAMP level indicates a Th17 mediated disease. In embodiments, when the patient sample has a lower cAMP level compared to a control, the patient is administered an effective amount of a cAMP-elevating agent to treat the symptoms of the Th2-mediated disease. In embodiments, a lower cAMP level when compared to a control indicates a Th2 response resulting from an infection, such as a parasitic or helminthic infection. In such embodiments, a cAMP-lowering agent is administered to the patient to promote a pro-Th2 response. In embodiments, when the patient sample has a higher cAMP level compared to a control, the patient is administered an effective amount of a cAMP-lowering agent to treat the symptoms of the Th17-mediated disease. The cAMP-elevating agent and cAMP-lowering agent are as described herein, including embodiments thereof. The patient sample may be a dendritic cell taken from the patient. The patient sample may be a blood drawn sample, wherein the cAMP level is in APCs found in the blood. The detection may occur after activation of a Gαs or Gαi pathway using an agonist or antagonist as described herein.

### IV. Methods of Preventing a Th2 or Th17 Disease

In another aspect is a method of preventing a Th2-mediated disease in a patient in need thereof. The method includes administering to the patient an effective amount of a cAMP-elevating agent and an adjuvant. The cAMP-elevating agent may increase the intracellular levels of cAMP in an APC. The Th2-mediated disease is as described herein. The APC is as described herein, including embodiments thereof. The cAMP-elevating agent is as described herein, including embodiments thereof. The adjuvant is as described herein, including embodiments thereof. The adjuvant may be alum. The cAMP-elevating agent may be absorbed or bound to alum. The adjuvant may be an oil emulsion. The adjuvant may be a nanoparticle, wherein the nanoparticle is bound to the cAMP-elevating agent. The adjuvant may be a nanoparticle, wherein the cAMP-elevating agent is enclosed in the core of the nanoparticle. The adjuvant may be a liposome. The liposome may be capable of targeting APCs described herein and deliver the cAMP-elevating agent to the APC. The cAMP-elevating agent and the adjuvant may be a component of a vaccine. In embodiments, the cAMP-elevating agent is bound to the adjuvant. The adjuvant may be an antigen or an allergen. The cAMP-elevating agent may be conjugated to the adjuvant using conjugation chemistry as described herein, including embodiments thereof. The cAMP-elevating agent and the adjuvant may be co-administered to stimulate immunity. The co-administration may be accomplished via vaccination.

In another aspect is a method of preventing a Th2-mediated disease in a patient in need thereof. The method includes administering to the patient an effective amount of a cAMP-lowering agent and an adjuvant. The cAMP-lowering agent may decrease the intracellular levels of cAMP in an APC. The Th2-mediated disease is as described herein. The APC is as described herein, including embodiments thereof. The cAMP-lowering agent is as described herein, including embodiments thereof. The adjuvant is as described herein, including embodiments thereof. The adjuvant may be alum. The cAMP-lowering agent may be absorbed or bound to alum. The adjuvant may be an oil emulsion. The adjuvant may be a nanoparticle, wherein the nanoparticle is bound to the cAMP-lowering agent. The adjuvant may be a nanoparticle, wherein the cAMP-lowering agent is enclosed in the core of the nanoparticle. The adjuvant may be a liposome. The liposome may be capable of targeting APCs described herein and deliver the cAMP-lowering agent to the APC. The cAMP-lowering agent and the adjuvant may be a component of a vaccine. In embodiments, the cAMP-lowering agent is bound to the adjuvant. The adjuvant may be an antigen or an allergen. The cAMP-lowering agent may be conjugated to the adjuvant using conjugation chemistry as described herein, including embodiments thereof. The cAMP-lowering agent and the adjuvant may be co-administered to stimulate immunity. The co-administration may be accomplished via vaccination.

In another aspect is a method of preventing a Th17-mediated disease in a patient in need thereof. The method includes administering to the patient an effective amount of a cAMP-lowering agent and an adjuvant. The cAMP-lowering agent may decrease the intracellular levels of cAMP in an APC. The Th17-mediated disease is as described herein. The APC is as described herein, including embodiments thereof. The cAMP-lowering agent is as described herein, including embodiments thereof. The adjuvant is as described herein, including embodiments thereof. The adjuvant may be alum. The cAMP-lowering agent may be absorbed or bound to alum. The adjuvant may be an oil emulsion. The adjuvant may be a nanoparticle, wherein the nanoparticle is bound to the cAMP-lowering agent. The adjuvant may be a nanoparticle, wherein the cAMP-lowering agent is enclosed in the core of the nanoparticle. The adjuvant may be a liposome. The liposome may be capable of targeting APCs described herein and deliver the cAMP-lowering agent to the APC. The cAMP-lowering agent and the adjuvant may be a component of a vaccine. In embodiments, the cAMP-lowering agent is bound to the adjuvant. The adjuvant may be an antigen or an allergen. The cAMP-lowering agent may be conjugated to the adjuvant using conjugation chemistry as described herein, including embodiments thereof. The cAMP-lowering agent and the adjuvant may be co-administered to stimulate immunity. The co-administration may be accomplished via vaccination.

### V. Methods for Identifying cAMP-Elevating or cAMP-Lowering Agents

In another aspect is a method of identifying a cAMP-elevating agent. The method includes contacting a test compound with an APC. The test compound is allowed to elevate cAMP levels in the APC thereby forming an activated-APC. An elevated level of cAMP in the activated-APC is detected thereby identifying a cAMP-elevating agent. In embodiments, the method includes a CD4 T cell present with the APC. The CD4 T cell may be a cell as described herein, including embodiments thereof (e.g. a CD4+ naïve cell or a Th1, Th2 or Th17 cell). The CD4 T cell may be a CD4+ naïve cell as described herein, including embodiments thereof. The CD4 T cell may be a Th1 cell as described herein, including embodiments thereof. The CD4 T cell may be a Th2 cell as described herein, including embodiments thereof. The CD4 T cell may be a Th17 cell as described herein, including embodiments thereof. The APC may be a macrophage or a dendritic cell as described herein, including embodiments thereof. The APC may be a part of an organism such, for example, a mammal. The organism may be a human.

In embodiments, the contacting is performed in the presence of an adjuvant. The adjuvant is as described herein, including embodiments thereof. The adjuvant may stimulate immunity upon vaccination. When the cAMP-elevating agent is contacted in the presence of an adjuvant, the cAMP-elevating agent may provide for greater stimulation of immunity upon vaccination than in the absence of the cAMP-elevating agent. In embodiments, the cAMP-elevating agent may be absorbed to the adjuvant. In embodiments, the cAMP-elevating agent may be covalently bound (e.g. using conjugate chemistry) the adjuvant. In embodiments, the method includes the addition of an antigen. The antigen may be covalently bound (e.g. using conjugate chemistry) to the cAMP-elevating agent. The increased stimulation of immunity may result from increased dendritic cell induction of Th17 cells through a Gαs/Gαi pathway. The increased stimulation of immunity may result from decreased dendritic cell induction of Th2 cells through a Gαs/Gαi pathway. The increased dendritic cell induction may result from changes in intracellular cAMP concentration levels that activate the dendritic cell thereby inducing Th17 lineage conversion as described herein. When the cAMP-elevating agent is contacted in the presence of an adjuvant, the method may further include detecting a cytokine produced from the activated-APC. The cytokine may be detected using techniques known in the art. The cytokine may be detected using an ELISA test. The cytokine detected may be IL-6. The elevated level of cAMP may change the cytokine production profile of the APC when compared to the activated-APC.

In another aspect is a method of identifying a cAMP-elevating agent in the presence of an adjuvant. The method includes contacting a test compound and an adjuvant with an APC. The test compound is absorbed to the adjuvant and allowed to elevate cAMP levels in the APC thereby forming an activated-APC. The activated-APC is contacted with a first mature CD4 T cell. The activated-APC is incubated with the first mature CD4 T cell for a period of time to allow the activated-APC to convert the lineage of the mature CD4 T cell into a second mature CD4 T cell. An elevated level of cAMP in the APC may be detected in combination with detection of a cytokine produced from the second mature T cell. In embodiments, the profile of the cytokines produced from the second mature T cell indicates stimulation of immunity. The cAMP-elevating agent is as described herein, including embodiments thereof. The adjuvant is as described herein, including embodiments thereof. In embodiments, the cAMP-elevating agent may be absorbed to the adjuvant. In embodiments, the cAMP-elevating agent may be covalently bound (e.g. using conjugate chemistry) the adjuvant. In embodiments, the method includes the addition of an antigen. The antigen may be covalently bound (e.g. using conjugate chemistry) to the cAMP-elevating agent. The APC is as described herein, including embodiments thereof. The CD4 T cell is as described herein, including embodiments thereof. The APC and/or CD4 T cell may be part of an organism, such as, for example a mammal. The organism may be a human. The first mature T cell and second mature cell are as described herein, including embodiments thereof. The first mature T cell may be a Th1 cell or a Th17 cell. The first mature T cell may be a Th2 cell. The second mature T cell may be a Th2 cell. The second mature T cell may be a Th17 cell.

In another aspect is a method of identifying a cAMP-elevating agent in an APC Gαs-knockout mouse. The method includes administering a test compound to a Gαs-knockout mouse. The test compound is allowed to elevate cAMP levels in the Gαs-knockout mouse. The elevated cAMP levels in the Gαs-knockout mouse are then detected. The test compound may be administered in combination with an adjuvant (e.g. co-administered). The adjuvant is as described herein, including embodiments thereof. The adjuvant may be alum. In embodiments, the test compound may be absorbed to the adjuvant. In embodiments, the test compound may be covalently bound (e.g. using conjugate chemistry) the adjuvant. In embodiments, the method includes the addition of an antigen. The antigen may be covalently bound (e.g. using conjugate chemistry) to the test compound. The detecting may include comparing the level of cAMP to a control. When the level is greater than the control, the compound is a cAMP-elevating agent. The APC is as described herein, including embodiments thereof. The APC may be a dendritic cell as described herein, including embodiments thereof. The APC may be a macrophage.

The detection of elevated cAMP levels may be performed by observing a phenotypic change of the mouse. The phenotypic change may be an inhibition of symptoms of a Th2-mediated disease (e.g. decreased airway inflammation). Thus, the phenotypic change may be a means to diagnose or treat symptoms of a Th2-mediated disease. Accordingly, when symptoms of a Th2-mediated disease are mitigated through observation of a phenotypic change described herein, the cAMP-elevating agent is therapeutic (i.e. capable of treating a Th2-mediated disease). The phenotypic change may be inhibition of a chronic Th2-mediated disease. The Th2-mediated disease is a disease described herein, including embodiments thereof. The method may provide for preclinical testing of therapeutic cAMP-elevating agents in vivo. The method may provide for preclinical testing of preventive cAMP-elevating agents in vivo (e.g. vaccines). The preclinical testing may provide for greater recognition of efficacious compounds in the Gαs-knockout mouse because the Gαs-knockout mouse displays a phenotype similar to human disease progression.

In another aspect is a method of identifying a cAMP-lowering agent. The method includes contacting a test compound with an APC. The test compound is allowed to lower cAMP levels in the APC thereby forming an activated-APC. A lowered level of cAMP in the activated-APC is detected thereby identifying a cAMP-lowering agent. In embodiments, the method includes a CD4 T cell present with the APC. The CD4 T cell may be a cell as described herein, including embodiments thereof (e.g. a CD4+ naïve cell or a Th1, Th2 or Th17 cell). The CD4 T cell may be a CD4+ naïve cell as described herein, including embodiments thereof. The CD4 T cell may be a Th1 cell as described herein, including embodiments thereof. The CD4 T cell may be a Th2 cell as described herein, including embodiments thereof. The CD4 T cell may be a Th17 cell as described herein, including embodiments thereof. The APC may be a macrophage or a dendritic cell as described herein, including embodiments thereof. The APC may be a part of an organism such, for example, a mammal. The organism may be a human. When the cAMP level is lower than the level of the control, the test compound is a cAMP-lowering agent.

In embodiments, the contacting is performed in the presence of an adjuvant. The adjuvant is as described herein, including embodiments thereof. The adjuvant may stimulate immunity upon vaccination. When the cAMP-lowering agent is contacted in the presence of an adjuvant, it may provide for greater stimulation of immunity upon vaccination that in the absence of the cAMP-elevating agent. The increased stimulation of immunity may result from increased dendritic cell induction of Th2 cells through a Gαs/Gαi pathway. The increased dendritic cell induction may result from changes in intracellular cAMP concentration levels that activate the dendritic cell thereby inducing Th2 lineage conversion. The increased stimulation of immunity may result from increased dendritic cell induction of Th17 cells through a Gαs/Gαi pathway. When the cAMP-lowering agent is contacted in the presence of an adjuvant, the method may further include detecting a cytokine produced from the activated-APC. The cytokine may be detected using techniques known in the art. The cytokine may be detected using an ELISA test. The cytokine detected may be IL-4. The lowered level of cAMP may change the cytokine production profile of the APC when compared to the activated-APC.

In another aspect is a method of identifying a cAMP-lowering agent in the presence of an adjuvant. The method includes contacting a test compound and an adjuvant with an APC. The test compound is absorbed to the adjuvant and allowed to decrease cAMP levels in the APC thereby forming an activated-APC. The activated-APC is contacted with a first mature CD4 T cell. The activated-APC is incubated with the first mature CD4 T cell for a period of time to allow the activated-APC to convert the lineage of the mature CD4 T cell into a second mature CD4 T cell. A decreased level of cAMP in the APC may be detected in combination with detection of a cytokine produced from the second mature T cell. In embodiments, the profile of the cytokines produced from the second mature T cell indicates stimulation of immunity. The cAMP-lowering agent is as described herein, including embodiments thereof. The adjuvant is as described herein, including embodiments thereof. In embodiments, the cAMP-lowering agent may be absorbed to the adjuvant. In embodiments, the cAMP-lowering agent may be covalently bound (e.g. using conjugate chemistry) the adjuvant. In embodiments, the method includes the addition of an antigen. The antigen may be covalently bound (e.g. using conjugate chemistry) to the cAMP-lowering agent. The APC is as described herein, including embodiments thereof. The CD4 T cell is as described herein, including embodiments thereof. The APC and/or CD4 T cell may be part of an organism, such as, for example a mammal. The organism may be a human. The first mature T cell and second mature cell are as described herein, including embodiments thereof. The first mature T cell may be a Th1 cell or a Th17 cell. The first mature T cell may be a Th2 cell. The second mature T cell may be a Th2 cell. The second mature T cell may be a Th17 cell.

In another aspect is a method of identifying a cAMP-lowering agent in an APC Gαs-knockout mouse. The method includes administering a test compound to a Gαs-knockout mouse. The test compound is allowed to lower cAMP levels in the Gαs-knockout mouse. The lowered cAMP levels in the Gαs-knockout mouse are then detected. The test compound may be administered in combination with an adjuvant (e.g. co-administered). The adjuvant is as described herein, including embodiments thereof. The adjuvant may be alum. In embodiments, the test compound may be absorbed to the adjuvant. In embodiments, the test compound may be covalently bound (e.g. using conjugate chemistry) the adjuvant. In embodiments, the method includes the addition of an antigen. The antigen may be covalently bound (e.g. using conjugate chemistry) to the test compound. The APC is as described herein, including embodiments thereof. The APC may be a dendritic cell, including embodiments thereof. The APC may be a macrophage. The detection of lowered cAMP levels may be performed by observing a phenotypic change of the mouse. The phenotypic change may be a progression of symptoms of a Th2-mediated disease (e.g. increased airway inflammation). The phenotypic change may be exacerbation of a chronic Th2-mediated disease. The Th2-mediated disease is a disease described herein, including embodiments thereof. The phenotypic change may be prevention of a Th17-mediated disease. The Th17-mediated disease is as described herein, including embodiments thereof. The method may provide for preclinical testing of therapeutic cAMP-lowering agents in vivo. The method may provide for preclinical testing of preventative cAMP-lowering agents in vivo (e.g. vaccines).

Detection may be performed by microarray analysis of GPCR expression. The GPCR expression of the Gαs-knockout mouse may be different from the GPCR expression in a wild-type mouse. In the presence of a cAMP-elevating agent, the GPCR expression of APCs in the Gαs-knockout mouse may indicate a decreased Th2 response and mediation of a Th2-mediated disease. In the presence of a cAMP-lowering agent, the GPCR expression of APCs in the Gαs-knockout mouse may indicate an increased Th2 response and/or exacerbation of a Th2-mediated disease. In embodiments, upon addition of a cAMP-lowering agent, the GPCR expression of APCs in the Gαs-knockout mouse may indicate an increased Th2 response and treatment of a disease responsive to Th2 (e.g. parasitic or helminthic infections). In embodiments, upon addition of a cAMP-lowering agent, the GPCR expression of APCs in the Gαs-knockout mouse may indicate a decreases Th17 response and treatment of a Th17-mediated disease.

In embodiments, the GPCR expression of the Gαs-knockout mouse before and after treatment with a cAMP-elevating agent may be different thereby indicating GPCRs involved in progression or regression of a Th2-mediated disease or a Th17-mediated disease. Similarly, the comparison of GPCR expression of the Gαs-knockout mouse before and after treatment with a cAMP-lowering agent may be different thereby indicating GPCRs involved in progression or regression of a Th2-mediated disease or a Th17-mediated disease.

Thus the comparison of the GPCR expression before and after treatment with a cAMP-elevating agent or a cAMP-lowering agent may provide a method for identifying molecular targets for treating Th2-mediated diseases. The comparison of the GPCR expression before and after treatment with a cAMP-elevating agent or a cAMP-lowering agent may provide a method for identifying molecular targets for treating Th17-mediated diseases.

The detection may be performed by microarray analysis of dendritic cell gene expression. The gene expression of the Gαs-knockout mouse may be different from the gene expression in a wild-type mouse. In the presence of a cAMP-elevating agent, the gene expression of APCs in the Gαs-knockout mouse may normalize compared to the wild-type thereby indicating a decreased Th2 response and mediation of a Th2-mediated disease. In the presence of a cAMP-elevating agent, the gene expression of APCs in the Gαs-knockout mouse may diverge compared to the wild-type thereby indicating an increased Th17 response and exacerbation of a Th17-mediated disease.

In the presence of a cAMP-lowering agent, the gene expression of APCs in the Gαs-knockout mouse may diverge compared to the wild-type thereby indicating an increased Th2 response and exacerbation of a Th2-mediated disease. In the presence of a cAMP-lowering agent, the gene expression of APCs in the Gαs-knockout mouse may normalize compared to the wild-type thereby indicating a decreased Th17 response and mediation of a Th17-mediated disease.

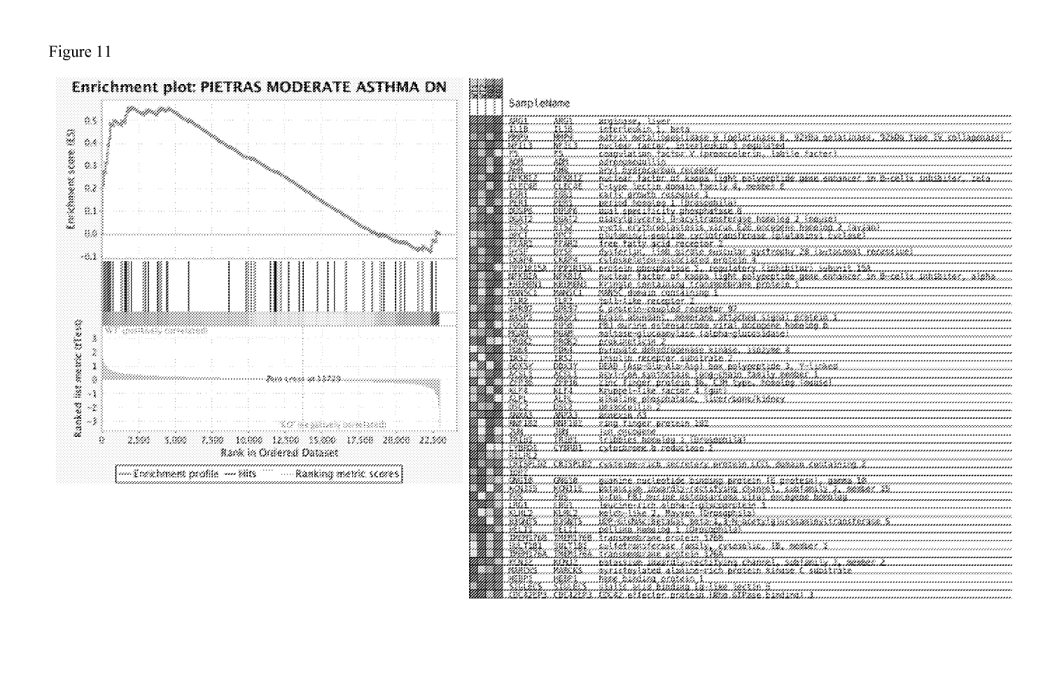

In embodiments, the genes are genes involved in the expression of proteins involved in the Gαs/Gαi pathway. In embodiments, the genes are those identified in Table 1, 2, 3, 4, 5, 6, 7, or in FIG. 11, 12, 13, or 20. In embodiments, the comparison of gene expression of the Gαs-knockout mouse before and after treatment with a cAMP-elevating agent or cAMP-lowering agent indicates genes involved in progression of the symptoms of a Th2-mediated disease. In embodiments, the comparison of gene expression of the Gαs-knockout mouse before and after treatment with a cAMP-elevating agent or cAMP-lowering agent indicates genes involved in progression of the symptoms of a Th17-mediated disease. Thus, the comparison of the gene expression before and after treatment with a cAMP-elevating agent or a cAMP-lowering agent may provide a method for identifying gene targets for treating a Th2-mediated disease or a Th17 mediated disease.

### VI. Knockout Mouse

In another aspect is a transgenic Gαs-knockout mouse having dendritic cells with a Gαs deletion (e.g. Gnas^{ΔCD11c}). The Gαs-knockout mouse may have CD11c+ cells with a Gαs deletion (e.g. Gnas^{ΔCD11c}) Progeny, ancestors, or cells of a parent Gαs-knockout mouse are also included herein. The Gαs-knockout mouse may be at an embryonic stage of development. The Gαs-knockout mouse may exhibit a Gαs/Gαi imbalance. The imbalance may result in a Th2 bias. The dendritic cells and bone marrow cells of the Gαs-knockout mouse may also exhibit a Gαs/Gαi imbalance. The Gαs-knockout may emulate genetic, immunological, or physiological features of human Th2-mediated diseases or Th17-mediated diseases. The Gαs-knockout mouse may emulate genetic features associated with human allergic diseases associated with Th2-response. In such embodiments, the Gαs-knockout mouse may serve as a preclinical test for evaluating test compounds to treat human allergic diseases. Similarly, the Gαs-knockout mouse may emulate immunological features of human Th2-mediated diseases. The Gαs-knockout mouse may emulate immunological features of human Th17-mediated diseases. The immunological features may be useful as a preclinical test for evaluating efficacy of test compounds to treat human allergic diseases mediated by Th2 response or inflammatory diseases mediated by Th17 response. The Gαs-knockout mouse may serve as a toxicology screen to determine toxicity of test compounds to treat human allergic diseases mediated by Th2 response, in vivo. The Gαs-knockout mouse may serve as a toxicology screen to determine toxicity of test compounds to treat human inflammatory disease mediated by Th17 response, in vivo. The Gαs-knockout mouse may emulate physiological features of human Th2-mediated diseases. The Gαs-knockout mouse may emulate physiological features of human Th17-mediated diseases. Such features may be observable as phenotypic changes. In embodiments, the knockout mouse is a conditional Gαs-knockout mouse.

Gnas^{ΔCD11c} mice are atopic, develop spontaneous Th2 response and a progressive chronic allergic phenotype that is akin to what occurs in patients with allergic asthma. The mouse may provide a method to identify effectors of Th2 differentiation. The mouse may provide a method to identify effectors of Th17 differentiation. Such effectors may be GPCRs, post-GPCR signaling proteins, cAMP-elevating or cAMP-lowering agents as described herein, or external signaling molecules effecting Th2 or Th17 differentiation. The mouse may facilitate discovery and testing of the effectors in an in vivo model that mimics human disease states. The mouse may serve as a means to analyze toxicity of therapeutics before entering early or late phase clinical trials.

In another aspect is a cell including a Gαs deletion (e.g. Gnas^{ΔCD11c}). In embodiments, the cell is a murine cell. In embodiments, the cell is an APC as described herein, including embodiments thereof. The APC may be a dendritic cell. The Gαs deletion may be a CD11c-specific deletion.

In another aspect is a method of producing a Gαs-knockout mouse. The method includes crossing a lox-flanked Gnas mouse with a CD11c-Cre or LysM-Cre mouse, wherein the Gαs-knockout mouse does not express Gαs. The non-expression of Gαs may be in dendritic cells or in macrophages.

### VII. Embodiments

### Embodiment 1

A method of inhibiting dendritic cell induction of CD4 T cell lineage conversion to a Th2 cell, said method comprising:

(i) contacting a dendritic cell with a cAMP-elevating agent in the presence of a CD4 T cell; and (ii) allowing cAMP concentration within said dendritic cell to increase relative to the absence of said cAMP-elevating agent thereby inhibiting dendritic cell induction of lineage conversion of said CD4 T cell to a Th2 cell, wherein said cAMP-elevating agent is exogenous to said dendritic cell

### Embodiment 2

The method of embodiment 1, wherein said cAMP-elevating agent comprises a Gαs-agonist, a PKA-agonist, a CREB-agonist, a cAMP analogue, a PDE inhibitor, a Gαi-antagonist, a GRK-antagonist, a RGS-antagonist, or a b-arrestin-antagonist

### Embodiment 3

The method of embodiments 1-2, wherein said dendritic cell forms part of an organism.

### Embodiment 4

The method of embodiments 1-3, wherein said CD4 T cell is a naïve CD4 T cell, a Th1 cell or a Th17 cell.

### Embodiment 5

A method of activating dendritic cell induction of CD4 T cell lineage conversion to a Th2 cell, said method comprising:

(i) contacting a dendritic cell with a cAMP-lowering agent in the presence of a CD4 T cell; and (ii) allowing cAMP concentration within said dendritic cell to decrease relative to the absence of said cAMP-lowering agent thereby activating dendritic cell induction of lineage conversion of said CD4 T cell to a Th2 cell, wherein said cAMP-lowering agent is exogenous to said dendritic cell.

### Embodiment 6

The method of embodiment 5, wherein said dendritic cell forms part of an organism.

### Embodiment 7

The method of embodiments 5-6, wherein said organism is a human or a mouse.

### Embodiment 8

The method of embodiments 5-7, wherein said cAMP-lowering agent comprises a Gαs-antagonist, a PKA-antagonist, a CREB-antagonist, a PDE activator, a Gαi-agonist, a GRK-agonist, a RGS-agonist, or a b-arrestin-agonist.

### Embodiment 9

The method of embodiments 5-8, wherein said CD4 T cell is a naïve CD4 T cell, a Th1 cell or a Th17 cell.

### Embodiment 10

A method of treating a Th2-mediated disease in a patient in need thereof, said method comprising administering to said patient an effective amount of a cAMP-elevating agent.

### Embodiment 11

The method of embodiment 10, wherein said cAMP-elevating agent comprises a Gαs-agonist, a PKA-agonist, a CREB-agonist, a PDE inhibitor, an adenylyl cyclase activator, a cAMP analogue, a Gαi-antagonist, a GRK-antagonist, a RGS-antagonist, or a b-arrestin-antagonist.

### Embodiment 12

The method of embodiments 10-11, wherein said Th2-mediated disease comprises allergic asthma, rhinitis, conjunctivitis, dermatitis, colitis, food allergy, insect venom allergy, drug allergy or anaphylaxis-prone conditions.

### Embodiment 13

The method of embodiments 10-12, wherein said method further comprises an antigen, an allergen or an adjuvant.

### Embodiment 14

The method of embodiments 10-13, wherein said antigen, said allergen, or said adjuvant is covalently bound to said cAMP-elevating agent.

### Embodiment 15

A method of inducing CD4 T cell lineage conversion using an APC, said method comprising:

(i) contacting an APC with a cAMP-lowering agent; (ii) allowing said cAMP-lowering agent to lower cAMP levels in said APC, thereby forming an activated-APC; (iii) contacting said activated-APC with a first mature CD4 T cell; (iv) allowing said activated-APC to convert the lineage of said first mature CD4 T cell into a second mature CD4 T cell, thereby inducing CD4 T cell lineage conversion using an APC.

### Embodiment 16

The method of embodiment 15, wherein said APC comprises a dendritic cell or a macrophage.

### Embodiment 17

The method of embodiments 15-16, wherein said mature CD4 T cell comprises a Th1 cell or Th17 cell.

### Embodiment 18

The method of embodiments 15-17, wherein said cAMP-lowering agent comprises a Gαs-antagonist, a PKA-antagonist, a CREB-antagonist, a PDE activator, a Gαi-agonist, a GRK-agonist, a RGS-agonist, or a b-arrestin-agonist.

### Embodiment 19

A method of identifying a cAMP-elevating agent, said method comprising:

(i) contacting a test compound with an APC; (ii) allowing said test compound to elevate cAMP levels in said APC thereby forming an activated-APC; (iii) detecting an elevated level of cAMP in said activated-APC thereby identifying a cAMP-elevating agent.

### Embodiment 20

The method of embodiment 19, wherein a CD4 T cell is present with said APC.

### Embodiment 21

The method of embodiments 19-20, wherein said CD4 T cell comprises a CD4+ naïve cell.

### Embodiment 22

The method of embodiments 19-21, wherein said CD4 T cell comprises a Th1 or Th17 cell.

### Embodiment 23

The method of embodiments 19-22, wherein said APC comprises a dendritic cell or a macrophage.

### Embodiment 24

A method for preventing a Th2-mediated disease, said method comprising administering to a patient an effective amount of a cAMP-elevating agent and an adjuvant.

### Embodiment 25

The method of embodiment 24, wherein said cAMP-elevating agent and said adjuvant are co-administered to stimulate immunity upon vaccination.

### Embodiment 26

The method of embodiments 24-25 further comprising an antigen or an allergen.

### Embodiment 27

The method of embodiments 24-26, wherein said antigen or said allergen is bound to said cAMP-elevating agent.

### Embodiment 28

The method of embodiments 24-27, wherein said cAMP-elevating agent is enclosed within a liposome, a microcapsule, or a nanoparticle.

### Embodiment 29

A method for preventing a Th17-mediated disease, said method comprising administering to a patient in need thereof, an effective amount of a cAMP-lowering agent and an adjuvant.

### Embodiment 30

The method of embodiment 29, wherein said cAMP-elevating agent and said adjuvant are co-administered to stimulate immunity upon vaccination.

### Embodiment 31

The method of embodiments 29-30 further comprising an antigen.

### Embodiment 32

The method of embodiments 29-31, wherein said antigen is bound to said cAMP-lowering agent.

### Embodiment 33

The method of embodiments 29-32, wherein said cAMP-lowering agent is enclosed within a liposome, a microcapsule, or a nanoparticle.

### Embodiment 34

A method of identifying a cAMP-elevating agent in an APC in a Gαs-knockout mouse, said method comprising:

(i) administering a test compound to a Gαs-knockout mouse; (ii) allowing said test compound to elevate cAMP levels in said Gαs-knockout mouse; and (iii) detecting said elevated cAMP levels in said Gαs-knockout mouse.

### Embodiment 35

The method of embodiment 34, wherein said APC comprises a dendritic cell.

### Embodiment 36

The method of embodiments 34-35, wherein said detecting comprises observing a phenotypic change of said Gαs-knockout mouse.

### Embodiment 37

The method of embodiments 34-36, wherein said phenotypic change comprises inhibition of a Th2 mediated disease or inhibition of a chronic Th2 mediated disease.

### Embodiment 38

The method of embodiments 34-37, wherein said Th2 mediated disease comprises allergic asthma, rhinitis, conjunctivitis, dermatitis, colitis, food allergy, insect venom allergy, drug allergy or anaphylaxis-prone conditions.

### Embodiment 39

A method of identifying a Th2-mediated disease having symptoms similar to a Th17-mediated disease, said method comprising

(i) detecting a cAMP level in a patient sample; and (ii) comparing said cAMP levels to a control thereby identifying a low cAMP level in said patient sample, thereby identifying a Th2-mediated disease.

### Embodiment 40

The method of embodiment 39, wherein said method further comprises activating a Gαs or a Gαi pathway in said sample.

### Embodiment 41

A conditional Gαs-knockout mouse comprising dendritic cells with a Gas deletion.

### Embodiment 42

The mouse of embodiment 42, wherein said mouse has a Th2 bias.

### Embodiment 43

A transgenic Gαs-knockout mouse comprising dendritic cells with a Gas deletion.

### Embodiment 44

The mouse of embodiments 41-43, wherein said deletion is a CD11c-specific Gαs deletion.

### Embodiment 45

A cell comprising a Gαs deletion.

### Embodiment 46

The cell of embodiment 45, wherein said cell is a murine cell.

### Embodiment 47

The cell of embodiments 45-46, wherein said cell is an APC.

### Embodiment 48

The cell of embodiments 45-47, wherein said cell is a dendritic cell.

### Embodiment 49

The cell of embodiment 45-48, wherein said Gαs deletion is a CD11c-specific deletion.

### Embodiment 50

A method of treating a Th2-mediated disease, said method comprising inhibiting gene targets identified by a micro array and comparing gene expression of said gene targets in wild type dendritic cells to gene expression of said gene targets in a Gαs-knockout dendritic cell.

### Embodiment 51

A method of treating a Th2-mediated disease by adoptive transfer of dendritic cells, wherein said dendritic cells comprise a cAMP-elevating agent or a cAMP-lowering agent.

### Embodiment 52

A method of identifying a Th2-mediated disease, said method comprising identifying gene targets by a micro array and comparing gene expression of said gene targets in wild type dendritic cells to gene expression of said gene targets in a Gαs-knockout dendritic cell.

### Embodiment 53

A method of treating a Th17-mediated disease, said method comprising inhibiting gene targets identified by a micro array and comparing gene expression of said gene targets in wild type dendritic cells to gene expression of said gene targets in a Gαs-knockout dendritic cell.

### Embodiment 54

A method of identifying a Th17-mediated disease, said method comprising identifying gene targets by a micro array and comparing gene expression of said gene targets in wild type dendritic cells to gene expression of said gene targets in a Gαs-knockout dendritic cell.

### Embodiment 55

A method of treating a Th17-mediated disease by adoptive transfer of dendritic cells, wherein said dendritic cells comprise a cAMP-lowering agent.

### Embodiment 56

A method of identifying a Th2-mediated disease, said method comprising identifying GPCR expression of a wild type mouse and comparing said GPCR expression to GPCR expression in a Gαs-knockout mouse, wherein said differential expression indicates GPCRs involved in progression of a Th2-mediated disease.

### Embodiment 57

The method of embodiment 56, wherein said method further comprises administration of a cAMP-elevating agent prior to comparing said GPCR expression in said Gαs-knockout mouse to said GPCR expression in said wildtype mouse.

### Embodiment 58

A method of identifying a Th17-mediated disease, said method comprising identifying GPCR expression of a wild type mouse and comparing said GPCR expression to GPCR expression in a Gαs-knockout mouse, wherein said differential expression indicates GPCRs involved in progression of a Th2-mediated disease.

### Embodiment 59

The method of embodiment 58, wherein said method further comprises administration of a cAMP-lowering agent prior to comparing said GPCR expression in said Gαs-knockout mouse to said GPCR expression in said wildtype mouse.

### Embodiment 60

A method of producing a Gαs-knockout mouse, said method comprising crossing a lox-flanked Gnas mouse with a CD11c-Cre or LysM-Cre mouse, wherein said Gαs-knockout mouse does not express Gαs.

### Embodiment 61

The method of embodiment 62, wherein said Gαs-knockout mouse does not express Gαs in dendritic cells or macrophages.

### Embodiment 63

A method of treating a Th2-mediated allergic disease, the method comprising administering a therapeutically effective dose of a cAMP agonist to a patient having the disease.

### Embodiment 64

The method of embodiment 63, wherein the patient has allergic asthma.

### Embodiment 65

The method of embodiments 63-64, wherein the patient has allergic asthma.

### Embodiment 66

A method of treating a Th2-mediated allergic disease, the method comprising administering a therapeutically effective dose of an agent that increases DC cAMP levels to a patient having the disease.

### Embodiment 67

The method of embodiment 66, wherein the patient has allergic asthma.

### Embodiment 68

A method of treating a Th17-mediate disease, the method comprising administering a therapeutically effective dose of an agent that decreases DC cAMP levels to a patient having the disease.

### Embodiment 69

A CD11c-specific GNAS KO mouse.

### Embodiment 70

A method of treating a patient that has a Th17-mediated inflammatory disease the method comprising administering a Gαs antagonist or Gαi agonist to the patient.

### Embodiment 71

The method of embodiment 70, wherein the patient has the allergic disease is allergic asthma, rhinitis, conjunctivitis, dermatitis, or a food allergy non-allergic asthma, Crohn's disease, multiple sclerosis, chronic obstructive pulmonary disease, or type-1 diabetes.

### Embodiment 72

A method of treating a patient that has a Th2-mediated allergic disease the method comprising administering a Gαs agonist or Gαi antagonist to the patient.

### Embodiment 73

The method of embodiment 60, wherein the allergic disease is allergic asthma, rhinitis, conjunctivitis, dermatitis, or a food allergy.

### Embodiment 74

A method of identifying a compound for the treatment of allergy diseases, asthma, the method comprising administering a candidate agent to a mouse of claim 3 and evaluating Th2, Th17 response in the mouse.

### VIII. Examples

### 1. Example 1

The role of dendritic cells (DC) in Th2 differentiation has not been fully defined. This gap in knowledge was addressed by focusing on signaling events mediated by the heterotrimeric (αβγ) GTP binding proteins Gαs, and Gαi, which respectively stimulate and inhibit the activation of adenylyl cyclases and synthesis of cAMP. Shown here, deletion of Gnas, the gene that encodes Gαs, in mouse CD11c⁺ cells and the accompanying decrease in cAMP provokes, whereas increases in cAMP by other means inhibits, progressive Th2 responses and an allergic phenotype. These findings imply that in addition to PRR, G-protein-coupled receptors, the physiological regulators of Gαs and Gαi activation and cAMP acting via PKA in DC affect Th bias and Th-mediated immunopathologies.

The induction of Th cell response requires APC, especially DC, but the mechanisms by which DC provoke Th2-type responses have not been elucidated¹⁻³. Furthermore, DC do not produce IL-4, a cytokine that is mandatory for GATA3 induction and Th2 cell differentiation^{4, 5}. These observations have suggested that other cell types are involved in Th2 responses^{1, 6, 7} including basophils⁸, epithelial cells⁹ and/or recently discovered innate immune helper cells¹⁰. Indeed, these cells can secrete IL-4 (basophils, innate immune helper cells) or alarmins such as IL-25, IL-33 and TSLP (epithelial cells), which support Th2 differentiation.

Pharmacological inhibition of members of the subfamily of phosphodiesterase 4 (PDE4), which is expressed highly in DC, were shown to improve animal models of inflammation and autoimmunity and to suppress human Th1-polarizing capacity through an increase in cAMP levels^{11, 12}. Based on these findings and previously published work that identified a role for cAMP levels in DC in Th17 induction¹³, another important signaling pathway in DC that affects Th differentiation bias is regulated by cAMP. To test this hypothesis, which involves a pathway not previously implicated in this context, the regulation of DC by heterotrimeric (αβγ) GTP binding proteins were studied that regulate cAMP synthesis through their modulation of the activity of adenylyl cyclases (ACs): Gαs, which stimulates and Gαi, and which inhibits membrane AC activity. In the current studies mice were engineered that have a CD11c-specific deletion of Gnas (CD11c-Cre Gnas^{fl/fl}, i.e., Gnas^{ΔCD11c}), the gene that encodes Gαs¹⁴. Gαs activation of CD11c⁺ cells from these mice generates much less cAMP than do equivalent cells from littermate controls. Unexpectedly, the Gnas^{ΔCD11c} mice display a striking and unique phenotype: they develop spontaneous Th2 immunity and Th2-mediated immunopathology even though this occurs on the C57Bl/6 genetic background¹⁵. DC from the Gnas^{ΔCD11c} mice display in vitro a pro-Th2 phenotype (i.e., they induce a Th2 response when co-cultured with CD4 T cells), which is reversed by exogenous administration of a cell-permeable cAMP analogue. Together with previous findings¹³, the current results identify a previously unappreciated role for Gαs-regulated cAMP synthesis and cAMP concentrations in DC in determining Th differentiation and resultant responses.

Generation of CD11c-Cre Gnas^{fl/fl} (Gnas^{ΔCD11c}) Mice

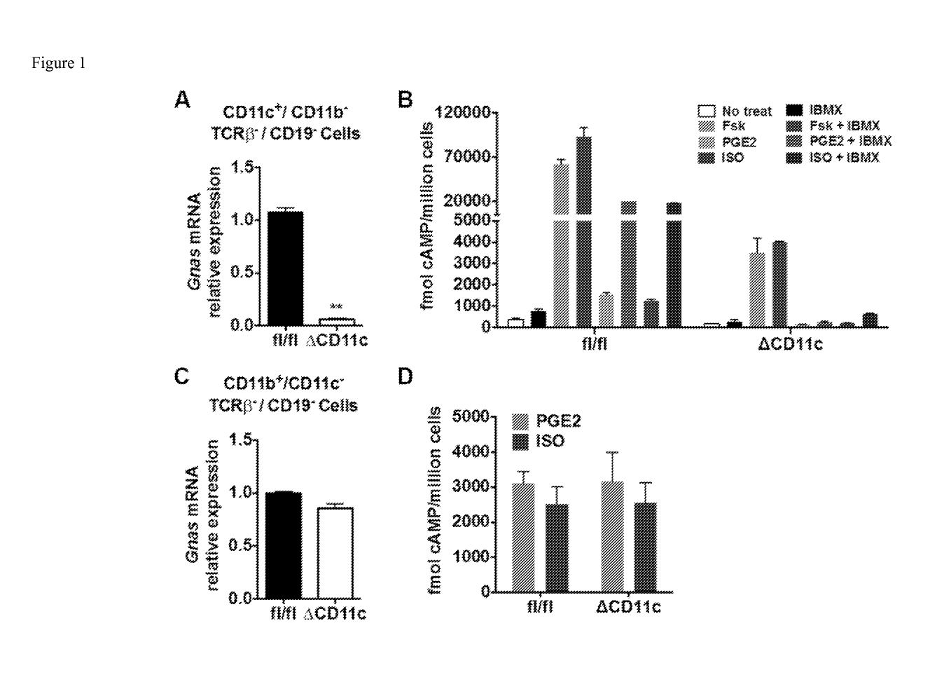

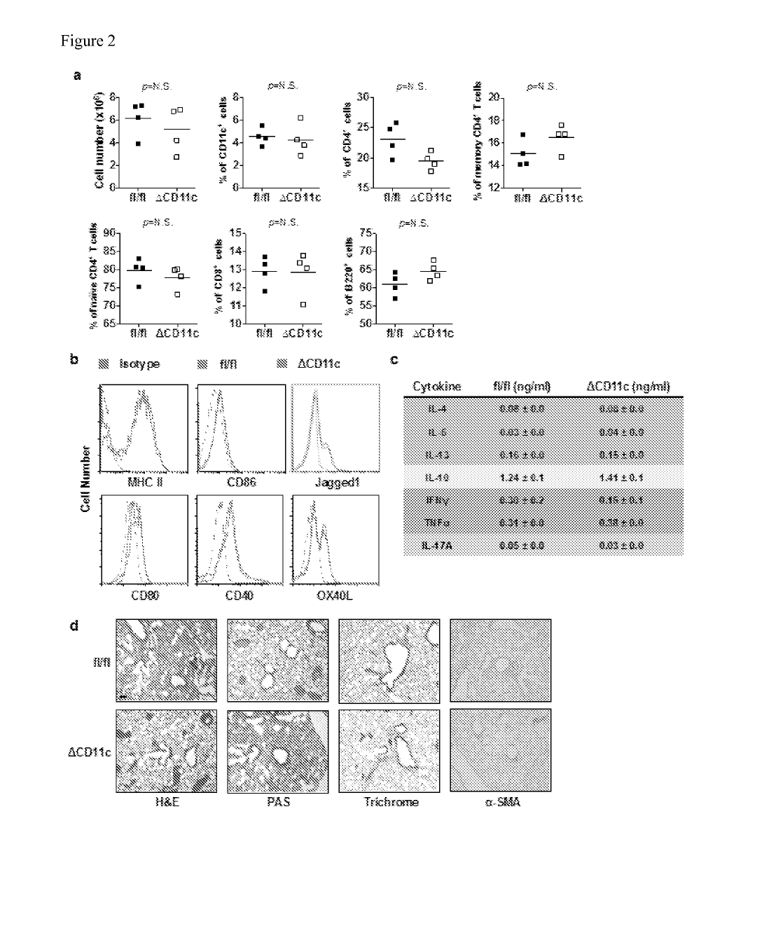

GPCR-mediated increase in intracellular cAMP requires the activation of AC by Gαs¹⁶. To obtain mice with DC deficient in this pathway, we used the Cre-loxP system to generate mice (B6 background) with a targeted deletion of Gnas in CD11c⁺ cells¹⁷. Splenic CD11c⁺/CD11b⁻ cells from these mice express low levels of Gnas mRNA and accumulate much less cAMP (FIGS. 1a and 1b) than do splenic CD11b⁺/CD11c⁻ cells (FIG. 1c, d). Gnas^{ΔCD11c} mice develop normally and have similar percentage of CD11c⁺, of CD4⁺ and CD8⁺ T cells, and of effector memory (CD44^{high}CD62L^{low}) and naïve (CD44^{low} CD62L^{high}) CD4⁺ T cells (FIG. 2) as do littermate (fl/fl) controls. Thus, the loss of Gnas does not significantly alter the number of peripheral DC or T cells.

Gnas^{ΔCD11c} Mice are Atopic and Develop Spontaneous Th2-Mediated Inflammation

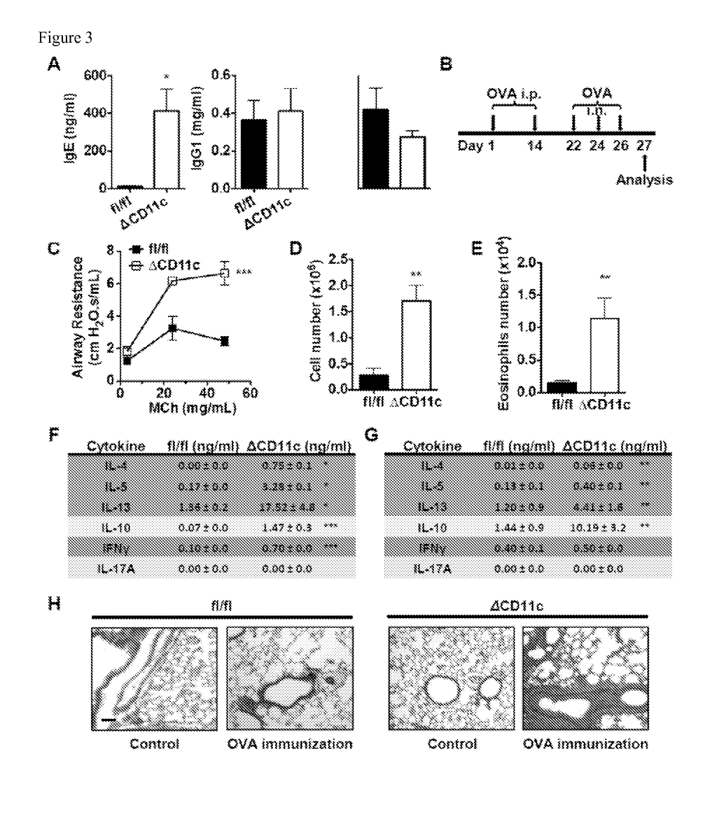

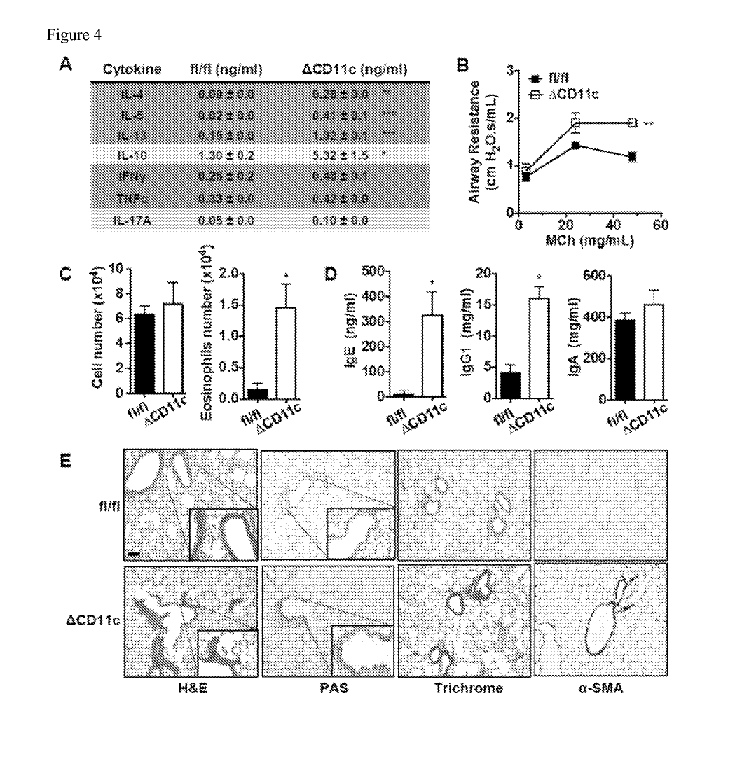

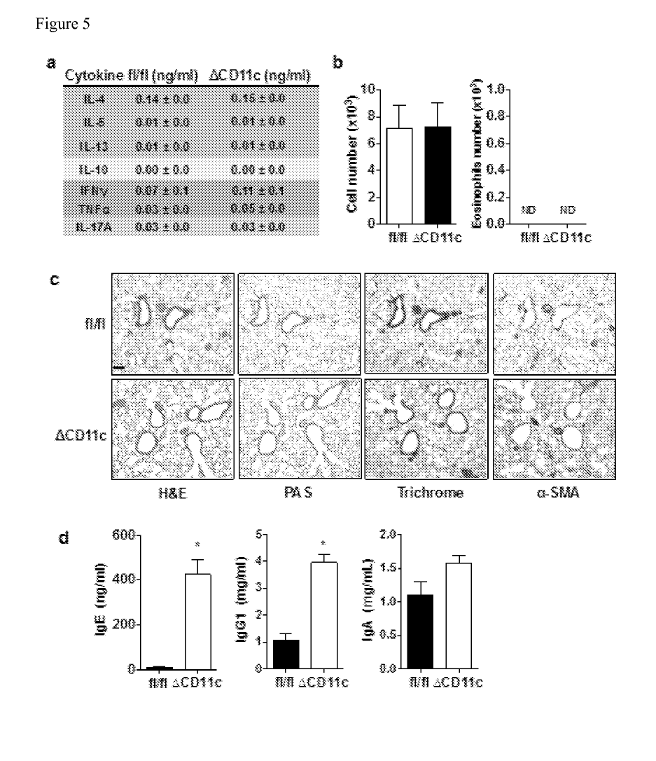

The CD4⁺ T cell cytokine profile of 2-month old Gnas^{ΔCD11c} mice is similar to that of co-housed littermate fl/fl mice (both on the B6 background), but serum IgE levels are increased in the Gnas^{ΔCD11c} mice (FIG. 3a). If Gnas^{ΔCD11c} mice were immunized even with a conventional antigen and challenged^{18, 19} they would develop Th2-mediated lung inflammation. Indeed, ovalbumin (OVA) immunization (without any adjuvant) provoked strong airway hyper-reactivity (AHR), an increased number of eosinophils in the bronchoalveolar lavage (BAL) fluid, increased Th2 cytokine response and airway inflammation in the Gnas^{ΔCD11c} but not in littermate fl/fl, mice (FIG. 3b-h). Moreover, 5-month old Gnas^{ΔCD11c} mice, but not littermate fl/fl mice, developed “spontaneous” Th2 response, i.e., without immunization (FIG. 4a), and displayed features of severe Th2-mediated lung inflammation (i.e., similar to those developed in experimental allergic asthma) that include AHR (FIG. 4b), increased number of eosinophils in the BAL fluid (FIG. 4c), increased serum IgE, IgG1 levels (FIG. 4d), and airway inflammation with evidence of airway remodeling (FIG. 4e). By contrast, despite their higher IgE serum levels, Gnas^{ΔCD11c} mice housed under specific pathogen-free (SPF) conditions at 5-6 month of age, like their fl/fl littermates did not develop Th2 bias and histologic lung abnormalities (FIG. 5). Collectively, these data indicate that the Gnas^{ΔCD11c} mice are atopic and poised to mount “spontaneous” Th2 bias responses.

BMDC from Gnas^{ΔCD11c} Mice Induce a Th2 Differentiation

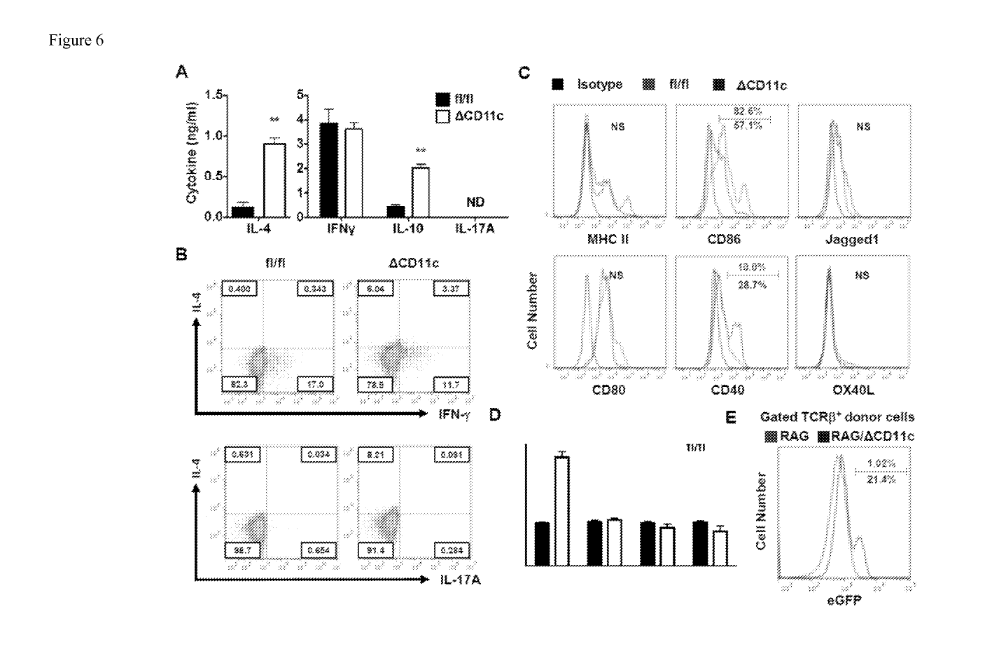

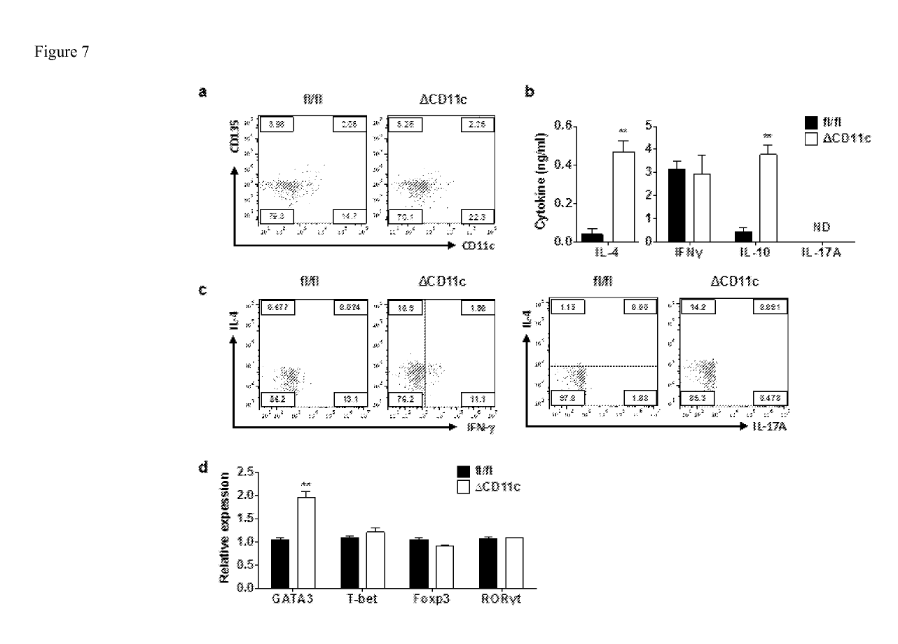

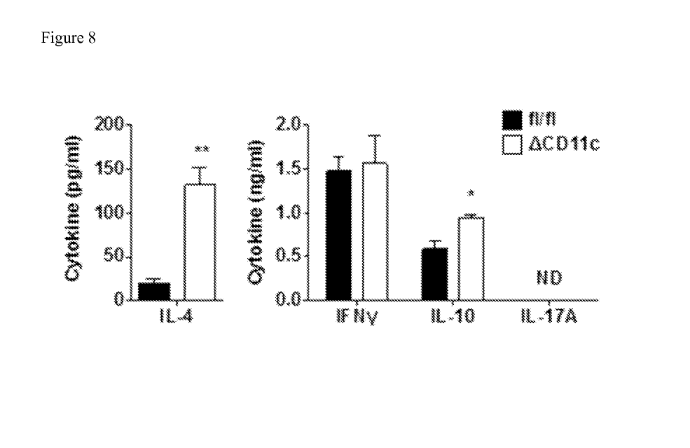

Intestinal and airway microbiota can affect Th differentiation and response. In vitro bone-marrow (BM) differentiated DC (BMDC) and naïve CD4 T cells were therefore used to further characterize the intrinsic role of BMDC from Gnas^{ΔCD11c} mice in Th2 bias. As a first approach, BM were cultured with GM-CSF and isolated CD11c⁺/Flt3⁺ double positive cells (i.e., BM-derived DC, BMDC) by FACS sorting^{20, 21}. These cells were then co-cultured with FACS-sorted naïve OT-2 splenic CD4⁺ T cells for 3 days. BMDC derived from Gnas^{ΔCD11c} mice (but not from littermate controls) induced high levels of IL-4 in the co-cultured OT-2 CD4⁺ T cells, as determined by ELISA (7-fold increase, FIG. 6a), or intracellular cytokine staining (13-fold increase, FIG. 6b). These BMDC also displayed an altered profile of expression of co-stimulatory molecules (FIG. 6c). Analysis of the Th lineage commitment factors of the OT-2 CD4⁺ T co-cultured with CD11c⁺/Flt3⁺ cells from Gnas^{ΔCD11c} mice revealed higher GATA3 levels (2.6-fold increase) (FIG. 6d), indicating that BMDC from Gnas^{ΔCD11c} mice have a pro-Th2 phenotype, i.e., they induce Th2 differentiation. CD11c⁺ single-positive BM cells from Gnas^{ΔCD11c} mice provoked a similar response (FIG. 7). Since GM-CSF-derived BMDC enhance development of inflammatory DC²², BM cultures were also stimulated with Flt3 ligand, which promotes development of plasmacytoid and conventional DC²⁰. BM-derived CD11c⁺ cells from Gnas^{ΔCD11c} (but not fl/fl) mice provoked a Th2 bias in the CD4⁺ T cell differentiation assay (FIG. 8). CD11c⁺/Flt3⁺ BM cells are a small fraction of the CD11c⁺ BM cells (Supplemental FIG. 7a) and because double-positive and the single-positive BM cells displayed a similar pro-Th2 phenotype, further in vitro analyses were undertaken using CD11c⁺ BM cells (i.e., single positive). Collectively, these in vitro data indicate that interaction of two cell types i.e., between BMDC and CD4⁺ T cells, is sufficient to provoke Th2 differentiation in this co-culture system.

As an additional means to assess Th2 differentiation in vivo we transferred naïve IL4-eGFP reporter (4get) CD4⁺ T cells^{23, 24} into Rag1^{-/-} or Rag1/Gnas^{ΔCD11c} double KO (DKO) mice and 3 weeks later analyzed eGFP fluorescence in splenic T cells. 21% of the reporter CD4⁺ T cells isolated from the DKO mice, but only 1% of those from the Rag1^{-/-} mice, were found eGFP⁺ (FIG. 6e). Taken together, the results indicate the crucial role of Gnas^{ΔCD11c} BMDC in the induction of Th2 bias.

PKA and Gαi Signaling Regulate the Induction of the Pro-Th2 Phenotype of CD11c⁺ BM Cells

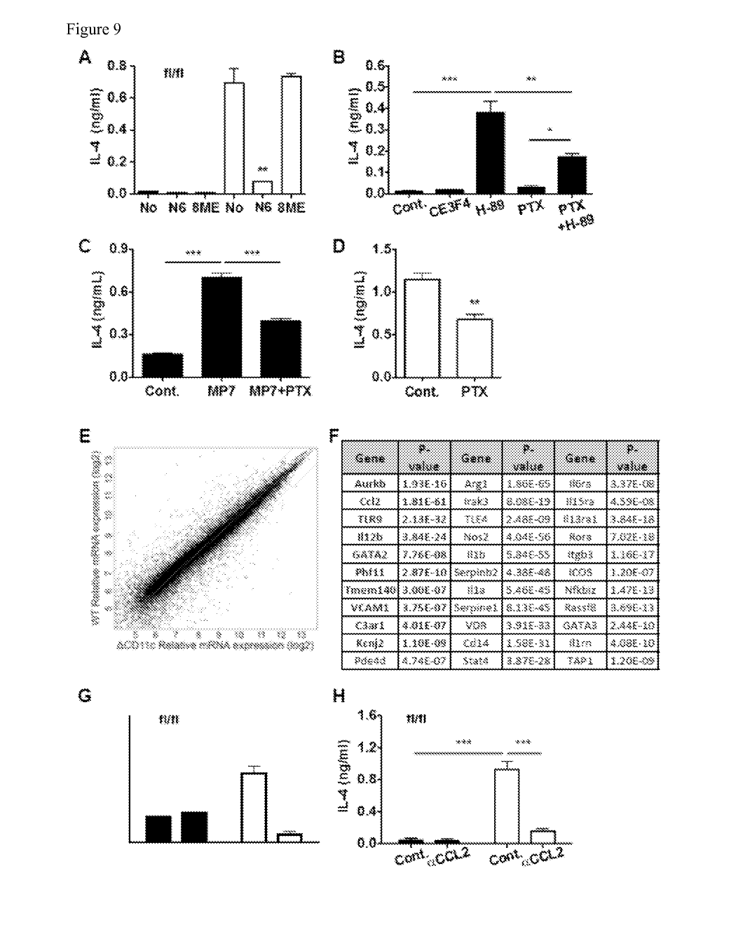

cAMP signaling pathway effectors were analyzed for their role in the pro-Th2 phenotype of CD11c⁺ BM cells isolated from Gnas^{ΔCD11c} mice. Cyclic AMP activates two main effector molecules, protein kinase A (PKA) and Exchange protein directly activated by cAMP (EPAC). Treatment with N6, a PKA-selective cAMP agonist, but not with 8ME, an EPAC agonist, abolished the pro-Th2 phenotype of Gnas^{ΔCD11c} CD11c⁺ BM cells (FIG. 9a). Furthermore, treatment of WT CD11c⁺ BM cells with a PKA inhibitor (H-89), but not with an EPAC inhibitor (CE3F4), promoted their pro-Th2 phenotype (FIG. 9b). These data implicate the important role of cAMP-PKA signaling pathway or its lack off in the inhibition or induction of the pro-Th2 phenotype of DC.

The deletion of Gnas in CD11c⁺ cells alters the balance between Gαs and Gαi in terms of cAMP synthesis and action with an increased potential impact of Gαi signaling. To assess this, WT CD11c⁺ BM cells were incubated with the Gαi activator mastoparan, a peptide toxin from wasp venom²⁵. Incubation with mastoparan 7 (MP7)²⁶ induced a pro-Th2 phenotype in WT CD11c⁺ BM cells. Moreover, incubation of MP7-treated or H-89-treated WT CD11c⁺ BM cells with pertussis toxin (PTX), which blocks Gi activation, inhibited this pro-Th2 phenotype (FIG. 9b, c). Additionally, inhibition of Gi signaling in Gnas^{ΔCD11c} CD11c⁺ BM cells with PTX (FIG. 9d) suppressed their pro-Th2 phenotype. Collectively, these results indicate that PKA signaling inhibits the pro-Th2 phenotype of both WT and Gnas^{ΔCD11c} CD11c⁺ BM cells and that activation of Gi contributes to the pro-Th2 phenotype of both types of cells. This result implies that the pro-Th2 phenotype in the Gnas-depleted CD11c⁺ DC reflects an altered balance between the activation of AC by Gαs and Gαi, and results from the subsequent decreases in cAMP concentration and reduced PKA activation in DC.

Genetic Similarities with Human Atopy and Allergic Asthma

Using DNA microarray, 2043 genes were found that were differentially expressed in CD11c⁺ BM cells of the Gnas^{ΔCD11c} mice compared to fl/fl mice (FIG. 9e). An enrichment and network analysis of the 717 genes that had >2-fold difference revealed that 33 differentially expressed genes in the Gnas^{ΔCD11c} mice match asthma-susceptibility genes identified in patient genome-wide association studies (GWAS)²⁷⁻²⁹ (FIG. 9f).

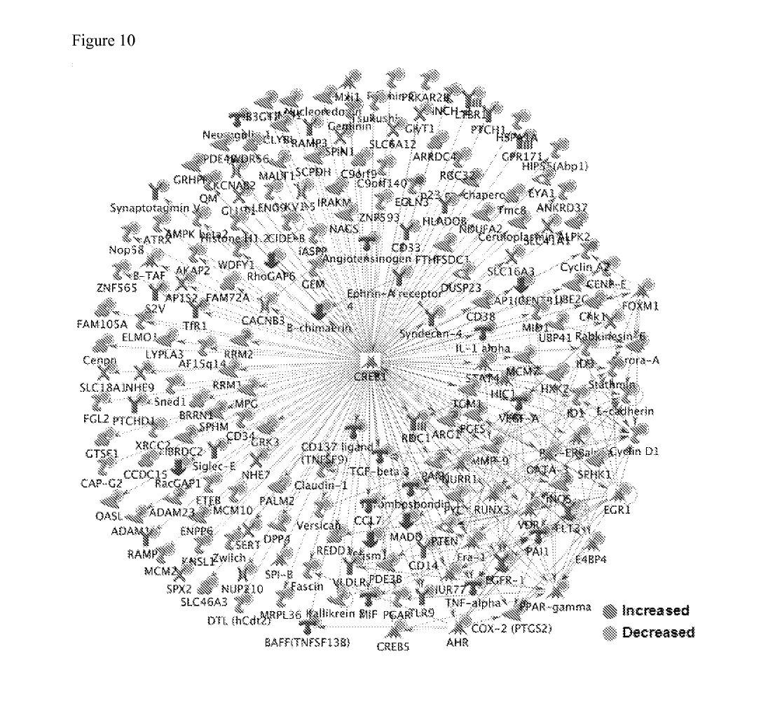

The pathway/process enrichment analysis highlighted that in addition to enrichment of immune response genes, ones involved in the cell cycle are enriched, suggesting that the decrease in Gαs expression and cellular cAMP concentration alter proliferation of CD11c⁺ cells in the Gnas^{ΔCD11c} mice (Tables 2 & 3). Network analysis of transcription factors identified CREB1 as the most important transcriptional regulator (Table 4): 29% of the differentially-regulated genes are CREB targets (FIG. 10) and 10 of the 33 GWAS genes are also CREB targets, suggesting altered expression or activity of cAMP/CREB-regulated proteins. expression of the transcript of CCL2 (MCP-1), a chemokine that activates the Gi-coupled GPCR, CCR2, was also found to be greater in Gnas^{ΔCD11c} CD11c⁺ BM cells, but if incubated with a cell-permeable cAMP analogue 8-CPT-cAMP (CPT), those cells have decreased CCL2 expression; moreover, addition of CCL2 neutralizing Ab inhibited their pro-Th2 phenotype (FIG. 9g, h).

TABLE 2
Pathway Enrichment Analysis of Genes altered in Gnas^{ΔCD11c} CD11c⁺ BM cells

| | | Total | | | Genes |
|---|---|---|---|---|---|
| # | Maps | Genes | pValue | FDR Corrected | In Data |
| 1 | Cell cycle: Chromosome condensation | 21 | 3.917E−10 | 2.0877E−07 | 10 |
| | in prometaphase | | | | |
| 2 | Cell cycle: Role of APC in cell | 32 | 6.732E−07 | 0.00017941 | 9 |
| | cycle regulation | | | | |
| 3 | PGE2 pathways in cancer | 55 | 1.600E−06 | 0.00028427 | 11 |
| 4 | Cell cycle: Start of DNA replication | 32 | 7.565E−06 | 0.00088413 | 8 |
| | in early S phase | | | | |
| 5 | Protein folding and maturation: | 43 | 9.783E−06 | 0.00088413 | 9 |
| | Angiotensin system maturation\ | | | | |
| | Human version | | | | |
| 6 | Immune response: CCL2 signaling | 54 | 9.953E−06 | 0.00088413 | 10 |
| 7 | Immune response: IL-1 signaling | 44 | 1.194E−05 | 0.00090922 | 9 |
| | pathway | | | | |
| 8 | Development: TGF-beta-dependent | 35 | 1.551E−05 | 0.00098165 | 8 |
| | induction of EMT via SMADs | | | | |
| 9 | Development: Hedgehog and PTH | 36 | 1.936E−05 | 0.00098165 | 8 |
| | signaling pathways in bone and | | | | |
| | cartilage development | | | | |
| 10 | Cell cycle: The metaphase checkpoint | 36 | 1.936E−05 | 0.00098165 | 8 |

TABLE 3
Process Enrichment Analysis of Genes altered in Gnas^{ΔCD11c} CD11c⁺ BM cells

| | | Total | | | Genes |
|---|---|---|---|---|---|
| # | Networks | Genes | pValue | FDR Corrected | In Data |
| 1 | Cell cycle: Core | 115 | 1.549E−09 | 2.4327E−07 | 24 |
| 2 | Cell cycle: S phase | 149 | 3.902E−09 | 3.0628E−07 | 27 |
| 3 | Development: Regulation of | 223 | 5.203E−08 | 2.7229E−06 | 32 |
| | angiogenesis | | | | |
| 4 | Cell cycle: G2-M | 206 | 1.141E−06 | 3.4986E−05 | 28 |
| 5 | Transport: Iron transport | 108 | 1.349E−06 | 3.4986E−05 | 19 |
| 6 | Inflammation: MIF signaling | 140 | 1.459E−06 | 3.4986E−05 | 22 |
| 7 | Cytoskeleton: Spindle microtubules | 109 | 1.560E−06 | 3.4986E−05 | 19 |
| 8 | Cell cycle: Mitosis | 179 | 2.685E−05 | 0.00052699 | 23 |
| 9 | Cell adhesion: Platelet-endo- | 174 | 1.499E−04 | 0.00238006 | 21 |
| | thelium-leucocyte interactions | | | | |
| 10 | Chemotaxis | 137 | 1.516E−04 | 0.00238006 | 18 |

TABLE 4
Transcription Factor Enrichment

| | | Total | Seed | | | |
|---|---|---|---|---|---|---|
| # | Network | nodes | nodes | p-Value | zScore | gScore |
| 1 | CREB1 | 209 | 208 | 0.000E+00 | 143.89 | 143.89 |
| 2 | SP1 | 145 | 144 | 8.200E−295 | 119.53 | 119.53 |
| 3 | c-Myc | 140 | 139 | 2.960E−284 | 117.42 | 117.42 |
| 4 | ESR1 | 111 | 110 | 1.820E−223 | 104.33 | 104.33 |
| 5 | GCR-alpha | 109 | 108 | 2.660E−219 | 103.37 | 103.37 |
| 6 | HIF1A | 107 | 106 | 3.850E−215 | 102.39 | 102.39 |
| 7 | p53 | 103 | 102 | 7.890E−207 | 100.42 | 100.42 |
| 8 | Oct-3/4 | 102 | 101 | 9.390E−205 | 99.92 | 99.92 |
| 9 | c-Jun | 98 | 97 | 1.850E−196 | 97.9 | 97.9 |
| 10 | E2F1 | 97 | 96 | 2.180E−194 | 97.39 | 97.39 |
| 11 | NF-kB (p65 RelA) | 94 | 93 | 3.520E−188 | 95.84 | 95.84 |
| 12 | AR | 90 | 89 | 6.500E−180 | 93.73 | 93.73 |
| 13 | E2F4 | 87 | 86 | 1.010E−173 | 92.11 | 92.11 |
| 14 | EGR1 | 84 | 84 | 1.590E−171 | 91.57 | 91.57 |
| 15 | C/EBPbeta | 86 | 85 | 1.160E−171 | 91.57 | 91.57 |
| 16 | NANOG | 83 | 82 | 1.760E−165 | 89.92 | 89.92 |
| 17 | YY1 | 81 | 80 | 2.290E−161 | 88.8 | 88.8 |
| 18 | STAT3 | 80 | 79 | 2.610E−159 | 88.23 | 88.23 |
| 19 | NF-kB1 (p50) | 78 | 77 | 3.370E−155 | 87.09 | 87.09 |
| 20 | PU.1 | 78 | 77 | 3.370E−155 | 87.09 | 87.09 |

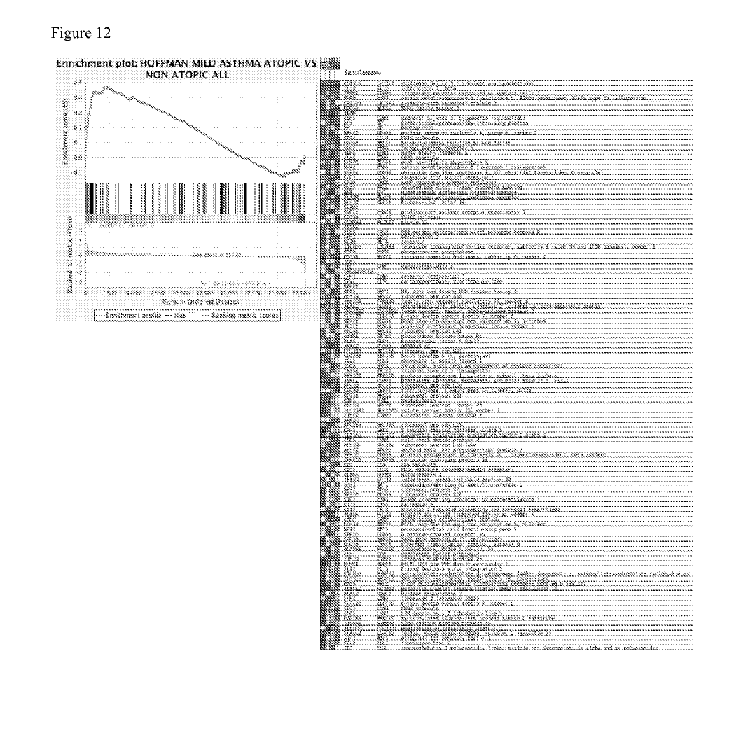

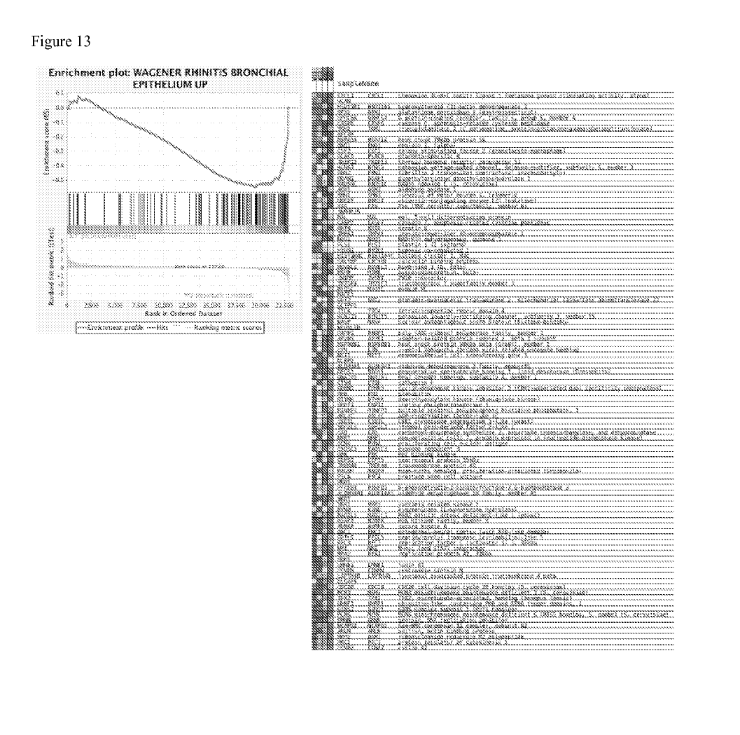

Using gene set enrichment analysis (GSEA), we compared our gene expression data to that of 7 human datasets from asthmatic, allergy and atopic subjects³⁰ (Table 5). CD11c⁺ BM Gnas^{ΔCD11c} mice express genes that are significantly enriched with ones found in 6 of 7 human studies (p<0.001, q<0.01, Table 6). CD11c⁺ BM cells of fl/fl mice show enrichment of genes that are down-regulated in WBC from asthmatic children (FIG. 11, GSE27011) and in atopic asthma compared to non-atopic asthma (FIG. 12, GSE473); in contrast, CD11c⁺ BM cells from the Gnas^{ΔCD11c} mice show enrichment of genes up-regulated in bronchial epithelia from subjects with allergic rhinitis (FIG. 13, GSE44037).

TABLE 5
Significantly enriched KEGG PATHWAYS by GSEA

| | | Enrichment | Normalized | | |
|---|---|---|---|---|---|
| GENESET NAME | SIZE | Score | Enrichment Score | p-value | FDR q-val |
| Enriched in WT DCs | | | | | |
| KEGG_GRAFT_VERSUS_HOST_DISEASE | 16 | 0.74109757 | 1.796272 | 0.003174603 | 0.106751 |
| KEGG_FRUCTOSE_AND_MANNOSE_METABOLISM | 32 | 0.608093 | 1.7188331 | 0.003115265 | 0.14461215 |
| KEGG_LEISHMANIA_INFECTION | 51 | 0.52840084 | 1.6630102 | 0.003039514 | 0.19203918 |
| KEGG_ARGININE_AND_PROLINE_METABOLISM | 48 | 0.5351404 | 1.639342 | 0.007215007 | 0.18503368 |
| KEGG_COMPLEMENT_AND_COAGULATION_CASCADES | 59 | 0.5095098 | 1.631794 | 0 | 0.16032346 |
| KEGG_GLYCOLYSIS_GLUCONEOGENESIS | 52 | 0.51794404 | 1.6150702 | 0.006144393 | 0.15645532 |
| KEGG_RENAL_CELL_CARCINOMA | 68 | 0.49370974 | 1.6034727 | 0.010401188 | 0.15048371 |
| KEGG_TYPE_1_DIABETES_MELLITUS | 21 | 0.594666 | 1.5490876 | 0.024509804 | 0.21246664 |
| KEGG_GALACTOSE_METABOLISM | 22 | 0.58260524 | 1.5290315 | 0.04040404 | 0.22713013 |
| KEGG_PENTOSE_PHOSPHATE_PATHWAY | 24 | 0.56059337 | 1.5123074 | 0.03514377 | 0.23469402 |
| KEGG_HEMATOPOIETIC_CELL_LINEAGE | 65 | 0.45924965 | 1.4979818 | 0.020408163 | 0.24220203 |
| Enriched in Gnas KO DCs | | | | | |
| KEGG_DNA_REPLICATION | 33 | −0.7790902 | −2.3915842 | 0 | 0 |
| KEGG_SPLICEOSOME | 98 | −0.5816171 | −2.2473936 | 0 | 0 |
| KEGG_CELL_CYCLE | 110 | −0.556181 | −2.1419923 | 0 | 0 |
| KEGG_RNA_DEGRADATION | 54 | −0.60468704 | −2.1064746 | 0 | 9.72E−04 |
| KEGG_MISMATCH_REPAIR | 20 | −0.6787328 | −1.8570107 | 0.005464481 | 0.027158773 |
| KEGG_NUCLEOTIDE_EXCISION_REPAIR | 37 | −0.5658251 | −1.8259711 | 0 | 0.029496253 |
| KEGG_HOMOLOGOUS_RECOMBINATION | 24 | −0.58339363 | −1.6972965 | 0.008547009 | 0.0886462 |
| KEGG_P53 SIGNALING_PATHWAY | 58 | −0.48789585 | −1.6880672 | 0.006134969 | 0.08355408 |
| KEGG_PROTEASOME | 43 | −0.48750538 | −1.597399 | 0.01764706 | 0.14182799 |
| KEGG_RENIN_ANGIOTENSIN_SYSTEM | 15 | −0.6116841 | −1.5641099 | 0.030470913 | 0.16303949 |
| KEGG_PYRIMIDINE_METABOLISM | 90 | −0.41069785 | −1.5629431 | 0 | 0.14968401 |
| KEGG_UBIQUITIN_MEDIATED_PROTEOLYSIS | 121 | −0.38089174 | −1.5058149 | 0.010948905 | 0.20573705 |

TABLE 6
Human asthma and/or atopy microarray datasets used for GSEA

| GEO | |
|---|---|
| Accession # | Description |
| GSE473 | Hoffman: CD4+ lymphocytes from 10 atopic controls, 10 non-atopic |
| | controls, 6 mild non-atopic asthmatics, & 41 mild atopic asthmatics |
| GSE15823 | Laprise: Bronchial biopsies from 4 asthmatics vs 4 healthy normal |
| GSE18965 | Beyer: AEC from 9 atopic asthma vs 7 healthy normal |
| GSE22528 | Laprise: BAL from 5 allergic asthma vs healthy normal |
| GSE27011 | Pietras: WBC from 20 severe & 20 mild asthma, & 19 normal children |
| GSE41649 | Chamberland: Bronchial biopsies from 4 atopic asthma vs 4 healthy normal |
| GSE44037 | Wagener: Bronchial and nasal biopsies from 6 subjects with rhinitis |
| | and asthma, 5 with allergic rhinitis, and 6 controls |

Adoptive Transfer of CD11c⁺ BM Cells from Gnas^{ΔCD11c} Mice Induces a Th2 Bias in WT Recipients and Increasing cAMP in Those Cells Blocks it

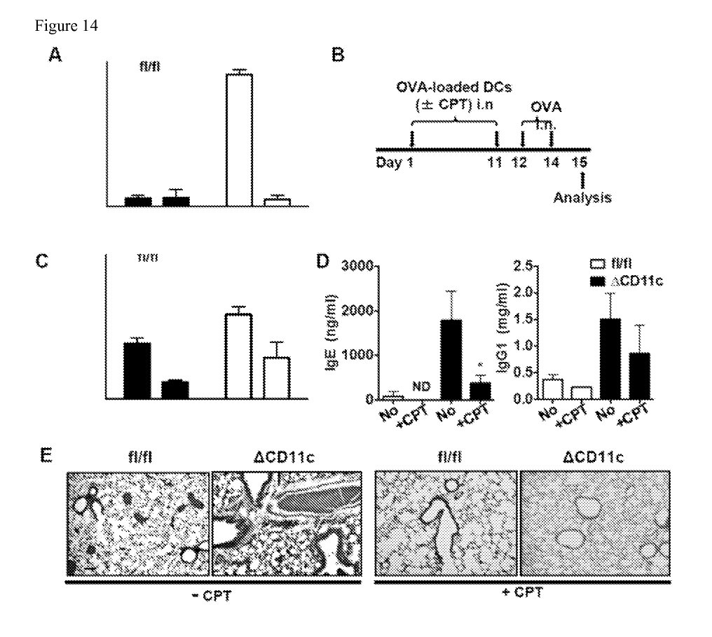

The data in FIG. 9a indicate that the administration of a PKA-specific cAMP agonist to Gnas^{ΔCD11c} BM cells inhibits their pro-Th2 phenotype. To further explore the possible inhibitory role of cAMP on Th2-mediated lung inflammation, these cells were treated in vitro with CPT. As shown in FIG. 14a, CPT treatment of Gnas^{ΔCD11c} BM cells abolished the subsequent IL-4 production by OT-2 CD4⁺ T cells in vitro. For in vivo testing we applied the protocol of adoptive transfer³¹ outlined in FIG. 6b. Intranasal transfer of OVA-loaded CD11c⁺ BM cells from Gnas^{ΔCD11c} mice induced OVA-specific IL-4 by splenic CD4⁺ T cells (FIG. 14c), higher levels of IgE (FIG. 6d) and airway inflammation (FIG. 14e) in both WT (B6) and Gnas^{ΔCD11c} recipients. However, treatment with CPT of Gnas^{ΔCD11c} CD11c⁺ BM cells in vitro prior to their transfer to recipient mice inhibited development of Th2 bias and airway inflammation in the recipients (FIG. 14c-e). Thus, an increase in cAMP concentration and signaling inhibits the pro-Th2 phenotype of CD11c⁺ BM cells from Gnas^{ΔCD11c} mice in vitro and in vivo.

Recent advances in innate and adaptive immunity have revealed the molecular basis of Th1, Th17 and Treg induction by DC′. These studies also showed the important role of activation of PRR by microbial products in the differentiation of the Th1/Th17 subsets^{33, 34}. In contrast, the mechanisms by which DC induce a Th2 response remain obscure, and thus, the involvement of other cell types in the induction of Th2 immunity has been proposed⁸⁻¹⁰. The data presented here indicate that a Gαs/Gαi signaling imbalance that favors Gi activation in BMDC provokes a Th2 response, which is reversed by increasing cellular cAMP content (FIG. 9). These data, combined with our previous observations that show induction of Th17 response¹³ by Gαs activation in BMDC, indicate that in addition to PRR, GPCR signaling via Gαs and Gαi in DC and potentially other APC is a critical contributor to Th subset differentiation.

Although Gnas^{ΔCD11c} mice are atopic from an early age (FIG. 2), conditions in which the mice are housed determine the spontaneous Th2 cytokine bias and induction of Th2-mediated inflammation (FIG. 4 and FIG. 5). The co-housed littermate control animals did not display these abnormalities under the two different housing conditions. Time-dependent effects of environmental stimuli thus contribute to the development and negative sequelae of Th2 response in the Gnas^{ΔCD11c} mice. These observations and the ability of CD11c⁺ BM cells derived from Gnas^{ΔCD11c} to provoke a Th2 response in vitro (FIG. 6 and FIG. 9) suggest that neither intestinal³⁵ nor airway³⁶ microbiota are necessary determinants of the induction of this Th2 bias. Therefore, gene-environment interaction appears to regulate Th2 differentiation and the subsequent development of the Th2-mediated immunopathologies³⁷ that occur in these animals.

The wasp venom-derived Gαi agonist mastoparan was found to induce the pro-Th2 DC phenotype in WT CD11c⁺ BM-derived cells and that Gi signaling, as observed by the blockade of that phenotype by treatment with PTX, suppresses this phenotype in WT cells (FIG. 9). Interestingly mastoparan derived from yellow jackets (Vespula vulgaris) shares similar activities³⁸ while melittin, the principal active component of bee venom has multiple biological activities that include Gi activation and Gs inhibition³⁹. Thus, the mechanism by which Hymenoptera venoms induce Th2 bias and allergy in humans may be via decreasing cAMP levels in DC at the sting areas of affected individuals. Furthermore, activation of PKA inhibits the pro-Th2 phenotype of Gnas^{ΔCD11c} BM cells while inhibition of PKA induces a pro-Th2 phenotype in WT CD11c⁺ BM cells (FIG. 9). These results indicate that a balance between Gs and Gi signaling appears to determine the pro-Th2 phenotype in both WT and Gnas^{ΔCD11c} DC. Consistent with this idea, transcriptomic analysis points to a key role of CREB in mediating cAMP effects to determine the pro-Th2 phenotype of DC.

Numerous animal models have been used to explore the pathogenesis of allergic disorders^{40, 41}. However, the failure to translate promising drug candidates that had been identified in such models to humans with those diseases leads one to question the utility of those models and emphasizes why new models are needed that more accurately reflect human immunology and genetics⁴². GWAS have identified genes involved in human allergy and asthma⁴³⁻⁴⁶. Comparison of genes differentially expressed by the Gnas^{ΔCD11c} mice to such human data reveals that expression of 33 human GWAS “hits” are altered in those mice, implying that DC may be critical in initiating or sustaining the allergic response. The similarity of the changes in gene expression in the Gnas^{ΔCD11c} mice to results obtained in 6 studies of human asthma/allergy supports this idea. Together, these findings imply that the immunogenetic changes observed in this mouse model are similar to those observed in humans and therefore suggest that these animals can help advance understanding and perhaps the treatment of allergic asthma in humans.

The increasing prevalence of allergic diseases during recent decades imposes significant public health challenges^{47, 48}. The prevalence of allergic diseases in the general population in the US is 22%; these diseases have an estimated annual health care-related cost of $30 billion⁴⁹. The pathophysiology of Gnas^{ΔCD11c} mice mimics that observed in allergic/asthmatic patients: Gnas^{ΔCD11c} mice are atopic, develop spontaneous Th2 response and a progressive chronic allergic phenotype that is akin to what occurs in patients with allergic asthma. These results imply that Gnas^{ΔCD11c} mice provide a unique system to identify novel molecular effectors of Th2 differentiation and their role in the induction of the allergic phenotype. In addition, this animal model may facilitate the discovery and testing of new therapeutics to prevent and treat allergy and asthma in humans. Based on the results shown here, we propose that targeting of DC-expressed GPCRs, the physiological activators of Gαs and Gαi (and thus regulators of cAMP formation) should provide such a therapeutic approach. Alternative means of influencing cAMP/PKA signaling can be envisaged but the wide utility and safety of drugs directed at GPCRs, including in the treatment of clinical features of allergic disorders, identify such receptors as particularly attractive targets for developing DC-directed therapy that will influence Th2 immunity.

Methods

Mice C57Bl/6 (B6) mice were purchased from Harlan Laboratories (Livermore, Calif.). CD11c-Cre transgenic mice and OT-2 (B6) were purchased from The Jackson Laboratory (Bar Harbor, Me.). To generate Gαs-deficient dendritic cells, lox-flanked Gnas²⁰ were crossed to CD11c-Cre mice. The CD11c⁺ cells in the Cre⁺Gnas^{ΔCD11c} mice were determined to be Gnas^{ΔCD11c}. The fl/fl littermates (Cre⁻Gnas^{fl/fl}) or B6 were used as control. Two to 6-month old mice were used in all the experiments.

Reagents

Reagents obtained are as follows: 8-(4-Chlorophenylthio) adenosine 3′,5′-cyclic monophosphate sodium salt (8-CPT-cAMP), forskolin, PGE2, isoproterenol, OVA albumin, and pertussis toxin (PTX) were from Sigma-Aldrich; Anti-mouse fluorescent labeled antibodies, anti-CCL2 antibody, and CD28 antibody from eBioscience; anti-mouse CD3e (clone 2C11) antibodies from BioXcell; Flt3 ligand from Peprotech; PKA inhibitor (H-89) from Calbiochem, N6 (PKA-specific cAMP analog, Phenyladenosine-3′,5′-cyclic monophosphate) and 8ME (EPAC-specific cAMP analog, 8-(4-Chlorophenylthio)-2′-O-methyladenosine-3′,5′-cyclic monophosphate) from Biolog; Mastoparan 7 (MP7) from Anaspec and EPAC inhibitor (CE3F4) was a gift from Dr. Frank Lezoualc'h (Université de Toulouse III Paul Sabatier, France).

Cyclic AMP Assay

Cyclic AMP accumulation was measured as previously described⁵⁰. Cells were prepared from sorted splenic CD11c⁺ (TCRb⁻CD19⁻CD11b⁻CD11c⁺) or CD11c⁻ (TCRb⁻CD19⁻CD11b⁺CD11c⁻) and equilibrated in RPMI 1640 medium containing 10% FCS for 30 min at 37° C. and then incubated with stimulatory agonists for 10 min in the absence and presence of PDE inhibitor 200 μM IBMX (added 30 min before the addition of agonists). Reactions were terminated by aspiration of the medium and addition of 50 μl of cold 7.5% (wt/vol) trichloroacetic acid (TCA) per million cells. Cyclic AMP content in TCA extracts was determined by radioimmunoassay and normalized to the amount of cells per well.

ELISA Measurement of Cytokines

CD4⁺ T cells were isolated by immunomagnetic selection (EasySep CD4⁺ negative selection kit, StemCell Technologies) from a single-cell suspension of splenocytes or peripheral lymph node cells. CD4⁺ T cells (1×10⁵ cells) were stimulated with plate-bound anti-CD3 Ab (10 μg/ml) and anti-CD28 Ab (1 μg/ml) for 24 h in complete RPMI medium (Mediatech Inc. Manassas, Va.) supplemented with 2 mM L-glutamine, 100 U/ml penicillin, 100 μg/ml streptomycin, 50 μM 2 β-mercaptoethanol, and 10% FCS. Cytokine levels in the supernatant were determined using ELISA kits for IL-4, IL-5, IL-13, IL-10, IFNγ, TNFα and IL-17A (eBioscience, La Jolla, Calif.) following the manufacturers' instructions as published¹⁷.

Measurement of Immunoglobulins

Serum was obtained and total IgE, IgG1, IgG2, and IgA levels were determined by ELISA, according to the manufacturer's instructions (Bethyl Laboratories, Inc. Montgomery, Tex.).

Flow Cytometry and Intracellular Staining

Antibodies used for cell labeling were purchased from BD PharMingen and eBiosciences. The data were acquired by a C6 Accuri flow cytometer (BD Biosciences) and analyzed by FlowJo Software. For measurements of intracellular cytokines, CD4⁺ T cells were stimulated with PMA (50 ng/ml) and ionomycin (1 μM) in the presence of GolgiStop (BD PharMingen) for 6 h. Cytokines were analyzed using fluorescent conjugated antibodies to IL-4, IL-17A, and IFNγ according to the manufacturer's instructions as published¹⁷.

OVA Immunization and Cytokine Measurement

WT and Gnas^{ΔCD11c} mice were injected intraperitoneally (i.p.) on day 1 and day 14 with OVA (50 μg, Sigma). On day 22, 24 and 26, the mice were intranasally challenged with OVA (20 μg). Animals were sacrificed and single-cell suspensions from bronchial lymph nodes and spleens were collected on day 27 and incubated for 3 days with media alone or supplemented with OVA (200 μg/mL). The concentration of cytokines in the supernatants was then determined (ELISA).

Determination of Airway Hyper-Responsiveness (AHR) to Methacholine (MCh)

AHR to MCh was assessed as described⁵¹ using intubated and ventilated mice (flexiVent ventilator; Scireq) anesthetized with ketamine (100 mg/kg) and xylazine (10 mg/kg) i.p. The frequency-independent airway resistance (Raw) was determined using Scireq software in mice exposed to nebulized PBS and MCh (3, 24, 48 mg/ml). The following ventilator settings were used: tidal volume (10 ml/kg), frequency (150/min), and positive end-expiratory pressure (3 cm H₂O) as previously published^{19, 52}.

Broncho-Alveolar Lavage (BAL) Fluid Analysis and Histological Evaluation of Lung

The lungs of mice of different conditions were equivalently inflated with 1 ml of PBS. This BAL fluid was spun down. The cells were counted and loaded on slides by cytospin for Giemsa Wright staining. BAL eosinophil counts were performed. For histological evaluation of the lung, 1 ml of 4% paraformaldehyde solution was injected intratracheally to preserve the pulmonary architecture. The inflated lungs were embedded in paraffin and tissue sections (5 μm) were prepared, deparaffinized and placed on slides. The slides were stained with hematoxylin-eosin for inflammatory cell infiltration, periodic acid Schiff (PAS) for identification of mucus-containing cells (goblet cells), Masson trichrome (MT) stain for peribronchiolar collagen, and immunostained for α-smooth muscle actin (α-SMA; DAKO, Glostrup, Denmark). They were examined using light microscopy and analyzed as previously described^{19, 52}.

OVA-Specific Immune Responses Upon In Vitro Co-Culture

BM cells were cultured in the presence of GM-CSF (10 ng/ml) for 7 days. For the analysis of double positive BM cells (i.e., BMDC), FACS-sorted CD11c⁺CD135⁺ BM cells from fl/fl and Gnas^{ΔCD11c} mice were treated with OVA for 24 h and then co-cultured (5×10⁵ cells) with naïve FACS-sorted OT-2 CD4⁺ T cells (1:1 ratio) for 3 days in complete PRMI 1640 medium (Invitrogen, Carlsbad, Calif.). The OT-2 cells were stimulated with plate-bound anti-CD3/28 Ab for 24 h and then used for ELISA to measure cytokines or stimulated by PMA and ionomycin for 4 h for intracellular staining. For the analysis of single positive BM cells, CD11c⁺ DC prepared from a single cell suspension of differentiated BM cells were isolated by magnetic beads (EasySep CD11c⁺ positive selection kit, StemCell Technologies). OT-2 T cells were isolated by use of CD4 magnetic beads (EasySep CD4⁺ negative selection kit, StemCell Technologies) from a single cell suspension of splenocytes. The DC from fl/fl and Gnas^{ΔCD11c} mice were treated with OVA for 24 h and then co-cultured (5×10⁵ cells) with the OT-2 T cells (1:1 ratio) and incubated for 3 days in complete PRMI 1640 medium (Invitrogen, Carlsbad, Calif.). The OT-2 T cells were stimulated with plate-bound anti-CD3/28 Ab for 24 h as described¹³.

For the inhibition of Th2 response by cAMP, fl/fl or Gnas^{ΔCD11c} BM-derived CD11c⁺ cells were cultured as above, then, incubated with 8-CPT-cAMP (50 μM) for 24 h, washed and then co-cultured with OT-2 T cells.

For the detection of cAMP signaling, fl/fl or Gnas^{ΔCD11c} BM-derived CD11c⁺ cells were cultured as above, then, incubated with N6 (PKA-specific cAMP analog, 50 μM) or 8ME (EPAC-specific cAMP analog, 50 μM) for 24 h, washed and then co-cultured with OT-2 T cells. WT BM-derived CD11c⁺ cells were cultured and then incubated with a PKA inhibitor (H-89, 10 μM), with or without pretreatment with pertussis toxin (PTX, 100 μg/ml, 18 h), or with EPAC inhibitor (CE3F4, 25 μM) for 30 min at 37° C., then washed and co-cultured with OT-2 T cells.

For the analysis of Gαi signaling, WT BM-derived CD11c⁺ cells were cultured and incubated with MP7 (1 μM) for 24 h, in the absence or presence of pretreatment with PTX, washed, and then incubated with OT-2 T cells. Gnas^{ΔCD11c} BM-derived CD11c⁺ cells were treated with pertussis toxin (PTX, 100 μg/ml, 18 h), washed and then incubated with OT-2 T cells.

Validation of the microarray data: fl/fl or Gnas^{ΔCD11c} BM-derived CD11c⁺ cells were cultured and then co-incubated with OT-2 T cells in the presence or absence of CCL2 neutralizing Ab (10 ng/ml). Flt3 ligand-stimulated BM cells: BM cell were cultured in the presence of Flt3 ligand (200 ng/ml) for 10 days as described⁵³, washed and then co-cultured with naïve OT-2 CD4⁺ T cells for 3 days (1:1 ratio).

Adoptive Transfer of 4Get CD4⁺ T Cells to Rag1^{-/-} Mice

Naive 4Get CD4⁺ T cells (CD4⁺CD45RB^{high}CD25⁻, 4×10⁵ cells/mouse) were sorted by FACS (BD Aria) and adoptively transferred i.p. into 6-month old sex- and age-matched Rag1^{-/-} or Rag1/Gnas^{ΔCD11c} DKO mice as described⁵⁴. Animals were sacrificed for analysis 3 week after transfer. Splenocytes were stimulated by PMA/ionomycin for 4 h in the presence of GolgiStop (BD PharMingen) for eGFP fluorescence.

Quantitative PCR Analysis

Isolation of RNA was carried out using an RNeasy Mini Kit (QIAGEN, Valencia, Calif.) according to the manufacturer's instructions. The cDNA was synthesized using Superscript III First-Strand system (Invitrogen). Quantitative PCR analysis was performed as described previously⁵⁴. SYBR Green PCR Master Mix was used for real-time PCR (7300 system, Applied Biosystems). Samples were run in triplicate and normalized by a housekeeping gene (mouse GAPDH). The primer sequences are provided in Table 7.

TABLE 7
Significantly enriched human genesets by GSEA

| | | Enrichment | Normalized | | |
|---|---|---|---|---|---|
| GENESET NAME | SIZE | Score | Enrichment Score | p-value | FDR q-val |
| Enriched in WT DCs | | | | | |
| PIETRAS: MODERATE ASTHMA DOWN | 59 | 0.55968714 | 1.7879491 | 0.00151286 | 0.01745721 |
| WAGENER: ASTHMA BRONCHIAL | 15 | 0.7035853 | 1.7144281 | 0.01244168 | 0.01867056 |
| EPITHELIUM UP | | | | | |
| LAPRISE: ASTHMA UP | 50 | 0.47032085 | 1.4498693 | 0.03886398 | 0.19237024 |
| MADORE: ASTHMA DOWN | 32 | 0.5068334 | 1.4389738 | 0.05909798 | 0.15614137 |
| WAGENER: ASTHMAS NASAL EPITHELIUM UP | 19 | 0.5549054 | 1.4178318 | 0.08156607 | 0.14921285 |
| WAGENER: RHINITIS BRONCHIAL | 112 | 0.39803922 | 1.4015304 | 0.02581522 | 0.13877445 |
| EPITHELIUM DOWN | | | | | |
| HOFFMAN: MILD ASTHMA ATOPIC VS | 115 | 0.47065252 | 1.6851027 | 0 | 0.017267266 |
| NON ATOPIC ALL | | | | | |
| HOFFMAN: ATOPY VS NON-ATOPIC CONTROLS ALL | 38 | 0.49962652 | 1.475723 | 0.036090225 | 0.06676678 |
| Enriched in Gnas KO DCs | | | | | |
| WAGENER: RHINITIS BRONCHIAL EPITHELIUM UP | 101 | −0.5498251 | −2.135054 | 0 | 0.00116783 |
| CHAMBERLAND: ATOPIC ASTHMA UP | 149 | −0.338516 | −1.39843 | 0.01526718 | 0.20713152 |
| WAGENER: RHINITIS NASAL EPITHELIUM DOWN | 23 | −0.4687382 | −1.3087511 | 0.1260997 | 0.23493439 |
| MADORE: ASTHMA UP | 18 | −0.4711056 | −1.281487 | 0.18387909 | 0.20525448 |
| ASTHMA GWAS GENES | 138 | −0.2987249 | −1.2133628 | 0.11328125 | 0.23253198 |

Adoptive Transfer of CD11c⁺ Cells to WT and Gnas^{ΔCD11c} Mice

Adoptive transfer of OVA-pulsed Gnas^{ΔCD11c} CD11c⁺ BM cells into mice was performed as described previously⁵⁵. BM cells were harvested from femurs and tibiae of Gnas^{ΔCD11c} mice and cultured in RPMI medium supplemented with 10% FCS, 10% penicillin-streptomycin, 2 mM L-glutamine, 50 μM 2-ME, and 10 ng/ml recombinant mouse GM-CSF for 1 week. CD11c⁺ BM cells were harvested from floating cells by use of a CD11c⁺ selection kit and loaded with OVA treated with or without 8-CPT-cAMP. After 24 h, CD11c⁺ cells were washed twice with PBS and resuspended in PBS. CD11c⁺ cells (2×10⁵) in 20 μl were transferred intranasally (i.n.) to recipients on days 1 and 11. The recipients were challenged by 30 μg OVA i.n. on days 12 and 14. 1 day after the last OVA challenge, mice were sacrificed and assessed by lung histology, measurement of serum immunoglobulins and cytokine production.

Transcriptome Analysis of BM-Derived CD11c⁺ Cells

CD11c⁺ BM cells from fl/fl and Gnas^{ΔCD11c} mice were cultured in the presence of GM-CSF for 7 days and then isolated by CD11c⁺ magnetic beads. Total RNA was harvested using RNAzolB (Tel-Test, TX) and purified on RNeasy spin columns (QIAGEN, Valencia, Calif.). The mRNA was quantified and its integrity checked by agarose gel electrophoresis. Messenger RNA (10 μg) from each culture was analyzed on Affymetrix mouse Gene 1.0 microarrays. Duplicates were run for each condition with independently isolated RNA from independent experiments. Genes showing differential regulation between conditions (Bonferroni corrected, α<0.05) were identified using Vampire and imported into MetaCore for pathway enrichment and network analysis¹³. To compare the results with human gene expression data, we analyzed 7 human asthma and atopy datasets (NIH GEO: GSE473, GSE15823, GSE18965, GSE22528, GSE27011, GSE41649, and GSE44037). Data from these studies were re-analyzed in Vampire using the same approach as used for the mouse profiling (FDR corrected, q<0.05). Lists of genes that were significantly up- or down-regulated in each dataset were generated and converted into GSEA genesets. The mouse data was then subjected to GSEA using the human asthma and atopy genesets. Enrichment of genesets in Gnas^{ΔCD11c} or fl/fl DCs was assessed.

Statistical Evaluation

Data are presented as mean±s.e.m. Unpaired Student's t-test with two-tailed p-values was used for statistical analyses unless indicated otherwise (Prism software). In all tests, P-values of less than 0.05 were considered statistically significant.

### REFERENCES

1. Pulendran, B. & Artis, D. New paradigms in type 2 immunity. Science 337, 431-435 (2012).
2. Zhu, J., Yamane, H. & Paul, W. E. Differentiation of effector CD4 T cell populations (*). Annu Rev Immunol 28, 445-489 (2010).
3. Coffman, R. L. Immunology. The origin of TH2 responses. Science 328, 1116-1117 (2010).
4. Kim, H. Y., DeKruyff, R. H. & Umetsu, D. T. The many paths to asthma: phenotype shaped by innate and adaptive immunity. Nat Immunol 11, 577-584 (2010).
5. Paul, W. E. & Zhu, J. How are T(H)2-type immune responses initiated and amplified? Nat Rev Immunol 10, 225-235 (2010).
6. Saenz, S. A., Noti, M. & Artis, D. Innate immune cell populations function as initiators and effectors in Th2 cytokine responses. Trends Immunol 31, 407-413 (2010).
7. Ohnmacht, C. et al. Basophils orchestrate chronic allergic dermatitis and protective immunity against helminths. Immunity 33, 364-374 (2010).
8. Voehringer, D. Protective and pathological roles of mast cells and basophils. Nat Rev Immunol 13, 362-375 (2013).
9. Holgate, S. T. Innate and adaptive immune responses in asthma. Nat Med 18, 673-683 (2012).
10. Scanlon, S. T. & McKenzie, A. N. Type 2 innate lymphoid cells: new players in asthma and allergy. Curr Opin Immunol 24, 707-712 (2012).
11. Schett, G., Sloan, V. S., Stevens, R. M. & Schafer, P. Apremilast: a novel PDE4 inhibitor in the treatment of autoimmune and inflammatory diseases. Ther Adv Musculoskelet Dis 2, 271-278 (2010).
12. Heystek, H. C., Thierry, A. C., Soulard, P. & Moulon, C. Phosphodiesterase 4 inhibitors reduce human dendritic cell inflammatory cytokine production and Th1-polarizing capacity. Int Immunol 15, 827-835 (2003).
13. Datta, S. K. et al. Mucosal adjuvant activity of cholera toxin requires Th17 cells and protects against inhalation anthrax. Proc Natl Acad Sci USA 107, 10638-10643 (2010).
14. Adams, G. B. et al. Haematopoietic stem cells depend on Gα(s)-mediated signalling to engraft bone marrow. Nature 459, 103-107 (2009).
15. Abbas, A. K., Murphy, K. M. & Sher, A. Functional diversity of helper T lymphocytes. Nature 383, 787-793 (1996).
16. Mosenden, R. & Tasken, K. Cyclic AMP-mediated immune regulation—overview of mechanisms of action in T cells. Cell Signal 23, 1009-1016 (2011).
17. Li, X. et al. Divergent requirement for Gαs and cAMP in the differentiation and inflammatory profile of distinct mouse Th subsets. J Clin Invest 122, 963-973 (2012).
18. Hayashi, T. et al. Induction and inhibition of the Th2 phenotype spread: implications for childhood asthma. J Immunol 174, 5864-5873 (2005).
19. Hayashi, T. et al. 3-Hydroxyanthranilic acid inhibits PDK1 activation and suppresses experimental asthma by inducing T cell apoptosis. Proc Natl Acad Sci USA 104, 18619-18624 (2007).
20. Merad, M., Sathe, P., Helft, J., Miller, J. & Mortha, A. The dendritic cell lineage: ontogeny and function of dendritic cells and their subsets in the steady state and the inflamed setting. Annu Rev Immunol 31, 563-604 (2013).
21. Hammer, G. E. & Ma, A. Molecular control of steady-state dendritic cell maturation and immune homeostasis. Annu Rev Immunol 31, 743-791 (2013).
22. Shortman, K. & Naik, S. H. Steady-state and inflammatory dendritic-cell development. Nat Rev Immunol 7, 19-30 (2007).
23. Mohrs, M., Shinkai, K., Mohrs, K. & Locksley, R. M. Analysis of type 2 immunity in vivo with a bicistronic IL-4 reporter. Immunity 15, 303-311 (2001).
24. McDonald, F., Mohrs, M. & Brewer, J. Using bicistronic IL-4 reporter mice to identify IL-4 expressing cells following immunisation with aluminium adjuvant. Vaccine 24, 5393-5399 (2006).
25. Lin, C. H., Tzen, J. T., Shyu, C. L., Yang, M. J. & Tu, W. C. Structural and biological characterization of mastoparans in the venom of Vespa species in Taiwan. Peptides 32, 2027-2036 (2011).
26. Jones, S. & Howl, J. Biological applications of the receptor mimetic peptide mastoparan. Curr Protein Pept Sci 7, 501-508 (2006).
27. Torgerson, D. G. et al. Meta-analysis of genome-wide association studies of asthma in ethnically diverse North American populations. Nat Genet 43, 887-892 (2011).
28. Gudbjartsson, D. F. et al. Sequence variants affecting eosinophil numbers associate with asthma and myocardial infarction. Nat Genet 41, 342-347 (2009).
29. Moffatt, M. F. et al. A large-scale, consortium-based genomewide association study of asthma. N Engl J Med 363, 1211-1221 (2010).
30. Hung, J. H., Yang, T. H., Hu, Z., Weng, Z. & DeLisi, C. Gene set enrichment analysis: performance evaluation and usage guidelines. Brief Bioinform 13, 281-291 (2012).
31. Lambrecht, B. N. & Hammad, H. Lung dendritic cells in respiratory viral infection and asthma: from protection to immunopathology. Annu Rev Immunol 30, 243-270 (2012).
32. Steinman, R. M. Decisions about dendritic cells: past, present, and future. Annu Rev Immunol 30, 1-22 (2012).
33. Joffre, O., Nolte, M. A., Sporri, R. & Reis e Sousa, C. Inflammatory signals in dendritic cell activation and the induction of adaptive immunity. Immunol Rev 227, 234-247 (2009).
34. Akira, S. Innate immunity and adjuvants. Philos Trans R Soc Lond B Biol Sci 366, 2748-2755 (2011).
35. Russell, S. L. & Finlay, B. B. The impact of gut microbes in allergic diseases. Curr Opin Gastroenterol 28, 563-569 (2012).
36. Huang, Y. J. et al. Airway microbiota and bronchial hyperresponsiveness in patients with suboptimally controlled asthma. J Allergy Clin Immunol 127, 372-381 e371-373 (2011).
37. Apter, A. J. Advances in adult asthma diagnosis and treatment in 2012: potential therapeutics and gene-environment interactions. J Allergy Clin Immunol 131, 47-54 (2013).
38. King, T. P., Jim, S. Y. & Wittkowski, K. M. Inflammatory role of two venom components of yellow jackets (Vespula vulgaris): a mast cell degranulating peptide mastoparan and phospholipase A1. Int Arch Allergy Immunol 131, 25-32 (2003).
39. Fukushima, N. et al. Melittin, a metabostatic peptide inhibiting Gs activity. Peptides 19, 811-819 (1998).
40. Takeda, K. & Gelfand, E. W. Mouse models of allergic diseases. Curr Opin Immunol 21, 660-665 (2009).
41. Holmes, A. M., Solari, R. & Holgate, S. T. Animal models of asthma: value, limitations and opportunities for alternative approaches. Drug Discov Today 16, 659-670 (2011).
42. Wenzel, S. & Holgate, S. T. The mouse trap: It still yields few answers in asthma. Am J Respir Crit Care Med 174, 1173-1176; discussion 1176-1178 (2006).
43. Zhang, Y., Moffatt, M. F. & Cookson, W. O. Genetic and genomic approaches to asthma: new insights for the origins. Curr Opin Pulm Med 18, 6-13 (2012).
44. Ober, C. & Yao, T. C. The genetics of asthma and allergic disease: a 21st century perspective. Immunol Rev 242, 10-30 (2011).
45. Kowal, K. et al. Analysis of −675 4 g/5 G SERPINE1 and C-159T CD14 polymorphisms in house dust mite-allergic asthma patients. J Investig Allergol Clin Immunol 18, 284-292 (2008).
46. Tamari, M., Tanaka, S. & Hirota, T. Genome-wide association studies of allergic diseases. Allergol Int 62, 21-28 (2013).
47. Pawankar, R., Canonica, G. W., Holgate, S. T. & Lockey, R. F. Allergic diseases and asthma: a major global health concern. Curr Opin Allergy Clin Immunol 12, 39-41 (2012).
48. Clark, N. M. Community-based approaches to controlling childhood asthma. Annu Rev Public Health 33, 193-208 (2012).
49. Akinbami, L. J. et al. Asthma outcomes: healthcare utilization and costs. J Allergy Clin Immunol 129, S49-64 (2012).
50. Murray, F. et al. Expression and activity of cAMP phosphodiesterase isoforms in pulmonary artery smooth muscle cells from patients with pulmonary hypertension: role for PDE1. Am J Physiol Lung Cell Mol Physiol 292, L294-303 (2007).
51. Lim, D. H. et al. Reduced peribronchial fibrosis in allergen-challenged MMP-9-deficient mice. Am J Physiol Lung Cell Mol Physiol 291, L265-271 (2006).
52. Song, D. J. et al. Toll-like receptor-9 agonist inhibits airway inflammation, remodeling and hyperreactivity in mice exposed to chronic environmental tobacco smoke and allergen. Int Arch Allergy Immunol 151, 285-296 (2010).
53. Mildner, A. et al. Mononuclear phagocyte miRNome analysis identifies miR-142 as critical regulator of murine dendritic cell homeostasis. Blood 121, 1016-1027 (2013).
54. Gonzalez-Navajas, J. M. et al. TLR4 signaling in effector CD4+ T cells regulates TCR activation and experimental colitis in mice. J Clin Invest 120, 570-581 (2010).
55. Machida, I. et al. Cysteinyl leukotrienes regulate dendritic cell functions in a murine model of asthma. J Immunol 172, 1833-1838 (2004).

### 2. Example 2

Adjuvants in Vaccinology:

Vaccination is a key tool in the protection against and eradication of infectious diseases and considered one of the most effective interventions that have impacted public health worldwide (1). Current human vaccines can be categorized into three general groups: modified live microorganism, killed/inactivated microorganism and subunit vaccines (a portion of the microorganism, toxins or toxoids). Each of these vaccine types has its advantages and disadvantages. Adjuvants—pharmacological or immunological agents that enhance antigen immunogenicity and/or modulate the type of immunity (e.g., humoral vs. cellular immune response)—are mainly used today in conjunction with subunit vaccines (2). The first adjuvant (alum) was introduced into clinical practice almost a century ago. In theory, an optimal vaccine should activate the two arms of the immune system; innate immunity (preferably dendritic cells) and adaptive immunity, including CD4 T cells, CD8 T cells and B cells. Effective adjuvants increase the immunogenicity of the co-injected antigen/immunogen by combining these immunological properties. Adjuvants enhance the immune response, provide protection against pathogens and thus are currently considered as an indispensable component of most clinically used subunit vaccines (3, 4). Because of this importance, the development of effective and safe adjuvants is significant for modern vaccinology.

Adjuvants and adjuvant systems function by one or several of the following mechanisms (based on Storni et al. (5):
Increasing antigen transport and uptake (phagocytosis) by antigen-presenting cells (APC) such as dendritic cells (DC)
Providing a long-lasting depot effect, i.e., antigenic reservoir for slow release
Triggering signal 0, e.g., efficient antigen processing and presentation, which precedes the induction of signal 1 mediated by MHC class I/II-TCR interaction
Triggering signal 2 (e.g., induction of co-stimulatory molecules and cytokine release by DC) necessary for the activation of naïve T cells
Provoking additional activation pathways such as pattern-recognition receptors (PRR, e.g, Toll-like receptors, TLR) or unfolded protein response (UPR)

Most current adjuvants do not have all of these functions. An effective adjuvant should address certain specific clinical needs and therefore should be tailored toward this objective. In this respect, an efficient adjuvant should be compatible with the delivery route (e.g., systemic vs. mucosal), provoke the desired immune response (e.g., humoral vs. cellular immunity), and address a particular stage of the required anti-microbial protection (e.g., preventive vs. therapeutic immunity). One way to achieve these diverse goals is to use a combination of complementary adjuvants (6). Certain adjuvant systems such as oil emulsions, adjuvant vesicles and liposomes are amenable to the inclusion of other adjuvants, such that their co-delivery customizes the adjuvanticity to address the clinical need. Indeed, a common practice in vaccination is to combine two synergistic adjuvants. These include, among others, TLR9 or TLR2 ligand within liposomes (7), alum adsorbed to TLR9 agonist (8), or MF59 mixed with TLR4 agonist (9). These complementary adjuvant combinations result in efficient, protective immune responses against the targeted pathogens and are used in clinical practice in some countries.

The Protective Role of Th17 in Infections:

Activation of naïve T cells by APC in the presence of signal 2 leads to the generation of distinct effector Th subsets that include Th1, Th2, and Th17. The Th1 subset regulates IFNγ-dependent immunity against intracellular pathogens. Th2 cells produce IL-4, IL-5 and IL-13, and are required for protection against helminths and certain parasitic infections. Th17 cells reside mainly in tissues that interface with the microbial environment, such as the gastrointestinal and respiratory tracts and the skin (10, 11). Th17-mediated protection against infectious agents is mediated by several synergistic mechanisms, including the release of antimicrobial peptides by epithelial cells, recruitment of neutrophils and macrophages at the site of infection, initiation of humoral immunity, and augmentation of other Th subsets. Epithelial cells, a main cellular target of Th17 cells, express receptors for Th17-derived cytokines. Triggering of epithelial cells by these cytokines results in the secretion of growth factors (e.g. G-CSF and GM-CSF) and chemokines (e.g. CXCL-1 and CCL2) that recruit neutrophils, DC and macrophages to the site of infection (10). Th17 cells are maintained as effector memory cells mainly in mucosal tissues for a very long period and display plasticity: the local cytokine milieu can switch their phenotype to Th1 or Th2-like cells. Although the phenotype of Th17 cells can be unstable under Th1 inflammatory conditions (12), stable long-lived memory Th17 cells are induced following vaccination in the absence of inflammation (12).

Th17 cells induce protective immunity against multiple bacterial and fungal pathogens (10, 13, 14). Vaccination in many mouse models of infectious diseases induces significant protective Th17 responses while neutralization of IL-17 or blockade of its downstream signaling results in higher pathogen burden and mortality. Th17 cells are required for clearance of S. pneumonia- and K. pneumonia-induced lung infections, eradication of Y. pestis, P. aeruginosa and protection against M. tuberculosis, B. pertussis, H. pylori and influenza virus. Th17 responses also provide protective immunity against fungal pathogens, including C. albicans, A. fumigatus B. dermatitidis, C. posodasii and H. capsulatum. A key part of this protection occurs by the recruitment and activation of DC, neutrophils and macrophages (10, 13).

Recall Response of Th17 Cells: Th17 Cell Plasticity:

In vitro and in vivo studies indicate that Th17 cells, which are characterized by IL-17A and/or IL-17F secretion, can convert to Th cells that secrete IL-17A and IFN-γ (double-positive cells), IFN-γ (Th1 cells), IL-22 (Th22 cells), and Treg cells. IL-22 targets epithelial surfaces (skin and mucosal layers) and enhances their defensive and barrier functions. Memory Th17 cells have been identified in both mice and humans; these cells express the Th17 lineage commitment transcription factor RORγt. However, the relative contributions of TGF-β, IL-2 IL-23 and IL-1β to Th17 memory response or plasticity differ in mouse vs. human. Overall these observations support the notion that Th17 cells serve as multi-potent, self-renewing precursors capable of differentiating into Th1-like effectors (Th17/Th1) and other progenies such as Th22 (10, 12) and Treg cells (10). Because Th1-like cells that originate from Th17 precursors lose their capacity for self-renewal and do not revert back to Th17 cells, they are considered more terminally differentiated and as such, have a lower survival rate than do the Th17 cells from which they arise. It has therefore been speculated that the greater self-renewing potential of Th17 cells relative to their Th1 progeny provides a long-lived pool of cells that can contribute to superior immune functions, such as those induced by vaccination with Th17 adjuvants, as we aim to discover in this proposal.

Th17 Adjuvants:

The induction of Th17 responses has been reported for non-alum-based adjuvants such as a nanoemulsions, incomplete Freund's adjuvants and MPL-trehalose dimycolate (15). The mucosal adjuvant, V. cholera-derived cholera toxin (CT), was discovered to induce Th17 responses in vivo and in vitro by DC through a cAMP/protein kinase A (PKA)-dependent mechanism (16). Use of E. coli-derived heat labile enterotoxin (LT) replicates this result (17). The major limitation of CT and LT usage is host toxicity. To overcome this drawback, recombinant cytokines, particularly IL-1β, IL-6 and IL-23, have been used as adjuvants. This strategy has been shown in pre-clinical models to increase the efficacy of Th17 induction (10).

The discovery herein that cAMP production in DC is critical in mediating Th17 adjuvanticity led to exploration of the role of cAMP-elevating compounds in the induction of Th17 response. The data herein indicate that a pharmacological approach, i.e., agents that can selectively activate cAMP/PKA in DC, are likely to act as powerful adjuvants with far better toxicity profiles than CT and LT, because the target action of such new agents would be limited to the critical cell type (i.e., DC) rather than occurring in most cells in the body, as observed for CT or LT. Furthermore, a focus on small (<1,000 Da MW), drug-like and non-immunogenic molecules (18) should eliminate immunogenicity problems associated with bacterial polypeptides (such as CT and LT) that raise cAMP levels via an irreversible mechanism.

Multiple cellular targets exist that can elevate cellular cAMP levels, including G protein-coupled receptors (GPCRs), regulators of G protein signaling (RGS), adenylyl cyclase isoforms, phosphodiesterases, and certain transporters. Importantly, many of these targets show differential expression among different cell types. In contrast, the single cellular target of CT and LT, the stimulatory Gα protein Gαs, is expressed ubiquitously (19-21). As outlined below, differential target expression provides an excellent situation for drug development, as it greatly improves the chances of identifying DC-selective agents that increase intracellular cAMP levels.

Immunization with cAMP-Elevating Agents Induces Th17 and Antibody Responses:

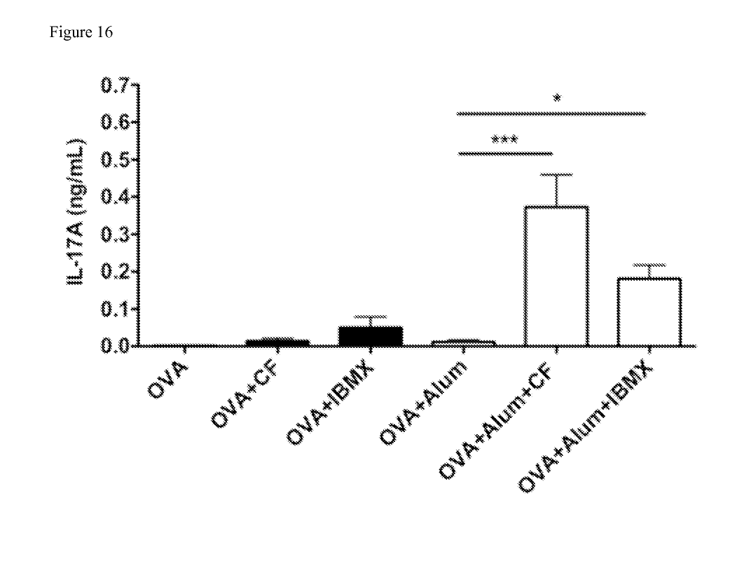

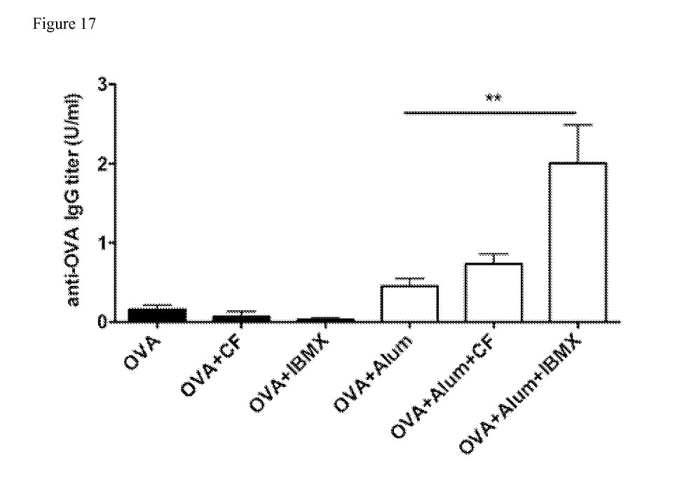

Based on the data presented above, co-immunization with antigen and a cAMP-elevating agent can induce a Th17 response. To promote stimulation of the same DC with antigen and cAMP agent, both were adsorbed to alum, an adjuvant used in humans (22), and immunized C57BL/6 (B6) mice with the combination. Both cAMP-elevating agents, colforsin and IBMX, provoked a robust OVA-specific IL-17 response in the presence of alum (FIGS. 16 and 17). These data support that agents that activate cAMP production and signaling pathways can be developed as powerful new adjuvants.

### REFERENCES

1. Nabel G J. 2013. Designing tomorrow's vaccines. The New England journal of medicine 368: 551-60
2. Fox C B, Haensler J. 2013. An update on safety and immunogenicity of vaccines containing emulsion-based adjuvants. Expert review of vaccines 12: 747-58
3. Awate S, Babiuk L A, Mutwiri G. 2013. Mechanisms of action of adjuvants. Frontiers in immunology 4: 114
4. Foged C. 2011. Subunit vaccines of the future: the need for safe, customized and optimized particulate delivery systems. Therapeutic delivery 2: 1057-77
5. Storni T, Kundig T M, Senti G, Johansen P. 2005 Immunity in response to particulate antigen-delivery systems. Advanced drug delivery reviews 57: 333-55
6. Mount A, Koernig S, Silva A, Drane D, Maraskovsky E, Morelli A B. 2013. Combination of adjuvants: the future of vaccine design. Expert review of vaccines 12: 733-46
7. Dow S. 2008. Liposome-nucleic acid immunotherapeutics. Expert opinion on drug delivery 5: 11-24
8. Li Y, Kandimalla E R, Yu D, Agrawal S. 2005. Oligodeoxynucleotides containing synthetic immunostimulatory motifs augment potent Th1 immune responses to HBsAg in mice. International immunopharmacology 5: 981-91
9. Singh M, Kazzaz J, Ugozzoli M, Baudner B, Pizza M, Giuliani M, Hawkins L D, Otten G, O'Hagan D T. 2012. MF59 oil-in-water emulsion in combination with a synthetic TLR4 agonist (E6020) is a potent adjuvant for a combination Meningococcus vaccine. Human vaccines & immunotherapeutics 8: 486-90
10. Weaver C T, Elson C O, Fouser L A, Kolls J K. 2013. The Th17 pathway and inflammatory diseases of the intestines, lungs, and skin. Annual review of pathology 8: 477-512
11. Rendon J L, Choudhry M A. 2012. Th17 cells: critical mediators of host responses to burn injury and sepsis. Journal of Leukocyte Biology 92: 529-38
12. Basu R, Hatton R D, Weaver C T. 2013. The Th17 family: flexibility follows function. Immunological reviews 252: 89-103
13. McGeachy M J, McSorley S J. 2012. Microbial-induced Th17: superhero or supervillain? Journal of immunology 189: 3285-91
14. Hernandez-Santos N, Gaffen S L. 2012. Th17 cells in immunity to Candida albicans. Cell host & microbe 11: 425-35
15. Kumar P, Chen K, Kolls J K. 2013. Th17 cell based vaccines in mucosal immunity. Current opinion in immunology 25: 373-80
16. Datta S K, Sabet M, Nguyen K P, Valdez P A, Gonzalez-Navajas J M, Islam S, Mihajlov I, Fierer J, Insel P A, Webster N J, Guiney D G, Raz E. 2010. Mucosal adjuvant activity of cholera toxin requires Th17 cells and protects against inhalation anthrax. Proc Natl Acad Sci USA 107: 10638-43
17. Norton E B, Lawson L B, Mandi Z, Freytag L C, Clements J D. 2012. The A subunit of Escherichia coli heat-labile enterotoxin functions as a mucosal adjuvant and promotes IgG2a, IgA, and Th17 responses to vaccine antigens. Infection and Immunity 80: 2426-35
18. Flower D R. 2012. Systematic identification of small molecule adjuvants. Expert opinion on drug discovery 7: 807-17
19. Antoni F A. 2012. New paradigms in cAMP signalling. Molecular and cellular endocrinology 353: 3-9
20. McDonough K A, Rodriguez A. 2012. The myriad roles of cyclic AMP in microbial pathogens: from signal to sword. Nature reviews. Microbiology 10: 27-38
21. Zaccolo M. 2011. Spatial control of cAMP signalling in health and disease. Current opinion in pharmacology 11: 649-55
22. Fierens K, Kool M. 2012. The mechanism of adjuvanticity of aluminium-containing formulas. Current pharmaceutical design 18: 2305-13

### 3. Example 3

Dendritic cells (DC) have a central role in the induction and polarization of Th subsets. Signaling events, which stimulate and inhibit the synthesis of cAMP in DC, play a role in modulating the pro-Th2 phenotype. GPCRs are the largest receptor family in the human genome, the sites of action for many hormones and neurotransmitters and the targets for over 30% of all prescription drugs. GPCRs are divided into four main classes according to the heterotrimeric G protein (Gα subunit) with which the receptors interact: Gαs, Gαi, Gαq/11, and Gα12/13, which each lead to the activation/inactivation of signaling pathways that control the production of second messengers, changes in activity of intracellular proteins and level of expression of various genes and proteins. GPCRs coupled to Gαs stimulate adenylyl cyclase (AC) and increase cellular cAMP concentrations, whereas Gαi inhibits AC activity, decreasing cAMP levels. This data indicates that CD11c-Cre Gnas fl/fl mice [mice with a CD11c-specific deletion of the gene that encodes the stimulatory Gα protein of the heterotrimeric (αβγ) GTP binding protein, Gαs have a Th2 bias, imply that Gαi-linked and Gαs-coupled GPCRs expressed by DC are targets to induce and regulate the induction of the Th2 response.

A mouse TaqMan® GPCR was used to identify and quantify GPCRs expressed in splenic DC and to determine if GPCR expression changes in DC from CD11c-Cre Gnas^{fl/fl} mice that show Th2 bias. Data indicated that global microarrays, such as those marketed by Affymetrix, that assess total cellular mRNA, are not optimal for detecting the cellular expression of GPCRs. The TaqMan® GPCR array detects 384 genes (355 non-chemosensory GPCRs and 29 housekeeping genes). WT splenic DC (CD11c+) were found to express 140 GPCRs.

Use of the GPCR array to assess DC from CD11c-Cre Gnas^{fl/fl} mice reveals that numerous GPCRs have increased, decreased or have unique expression in CLL cells. For example 5HT4 was a highly expressed Gαs-coupled GPCR in CD11c-Cre Gnas^{fl/fl} while CXCR4, was a highly expressed Gαi-coupled GPCR. CD11c-Cre Gnas^{fl/fl}-DC have an increase in those GPCRs that couple to Gαi, further enhancing the Gαi/Gαs bias.

Overall, these results indicate that GPCR profiling provides a very useful means to identify GPCRs that are expressed in DC, in particular those that could be targeted to increase cAMP and blunt Th2 polarization. Furthermore, these data show that CD11c-Cre Gnas^{fl/fl} DC have a Gαi/Gαs bias that favors Th2 induction. Thus, blockade of DC-expressed Gαi-linked GPCRs or enhanced signaling by Gαs-linked GPCRs may provide a strategy to regulate cAMP in DC hence affect different medical conditions/diseases. For example, the activation of Gαs and/or the inhibition of Gαi would be preferable to inhibit allergic/atopic/asthmatic disorders.

1. A method of inhibiting dendritic cell induction of CD4 T cell lineage conversion to a Th2 cell, said method comprising:
(i) contacting a dendritic cell with a cAMP-elevating agent in the presence of a CD4 T cell; and
(ii) allowing cAMP concentration within said dendritic cell to increase relative to the absence of said cAMP-elevating agent thereby inhibiting dendritic cell induction of lineage conversion of said CD4 T cell to a Th2 cell,
wherein said cAMP-elevating agent is exogenous to said dendritic cell.

2. The method of claim 1, wherein said cAMP-elevating agent comprises a Gαs-agonist, a PKA-agonist, a CREB-agonist, a cAMP analogue, a PDE inhibitor, a Gαi-antagonist, a GRK-antagonist, a RGS-antagonist, or a b-arrestin-antagonist.

3. (canceled)

4. A method of activating dendritic cell induction of CD4 T cell lineage conversion to a Th2 cell, said method comprising:
(i) contacting a dendritic cell with a cAMP-lowering agent in the presence of a CD4 T cell; and
(ii) allowing cAMP concentration within said dendritic cell to decrease relative to the absence of said cAMP-lowering agent thereby activating dendritic cell induction of lineage conversion of said CD4 T cell to a Th2 cell,
wherein said cAMP-lowering agent is exogenous to said dendritic cell.

5. (canceled)

6. The method of claim 4, wherein said cAMP-lowering agent comprises a Gαs-antagonist, a PKA-antagonist, a CREB-antagonist, a PDE activator, a Gαi-agonist, a GRK-agonist, a RGS-agonist, or a b-arrestin-agonist.

7. A method of treating a Th2-mediated disease in a patient in need thereof, said method comprising administering to said patient an effective amount of a cAMP-elevating agent.

8. (canceled)

9. The method of claim 7, wherein said Th2-mediated disease comprises allergic asthma, rhinitis, conjunctivitis, dermatitis, colitis, food allergy, insect venom allergy, drug allergy or anaphylaxis-prone conditions.

10. A method of inducing CD4 T cell lineage conversion using an APC, said method comprising:
(i) contacting an APC with a cAMP-lowering agent;
(ii) allowing said cAMP-lowering agent to lower cAMP levels in said APC, thereby forming an activated-APC;
(iii) contacting said activated-APC with a first mature CD4 T cell;
(iv) allowing said activated-APC to convert the lineage of said first mature CD4 T cell into a second mature CD4 T cell, thereby inducing CD4 T cell lineage conversion using an APC.

11. (canceled)

12. The method of claim 10, wherein said mature CD4 T cell comprises a Th1 cell or Th17 cell.

13. (canceled)

14. (canceled)

15. (canceled)

16. (canceled)

17. (canceled)

18. (canceled)

19. (canceled)

20. A method for preventing a Th2-mediated disease, said method comprising administering to a patient an effective amount of a cAMP-elevating agent and an adjuvant.

21. The method of claim 20, wherein said cAMP-elevating agent is enclosed within a liposome, a microcapsule, or a nanoparticle.

22. (canceled)

23. (canceled)

24. (canceled)

25. (canceled)

26. A method for preventing a Th17-mediated disease, said method comprising administering to a patient in need thereof, an effective amount of a cAMP-lowering agent and an adjuvant.

27. The method of claim 26, wherein said cAMP-elevating agent is enclosed within a liposome, a microcapsule, or a nanoparticle.

28. (canceled)

29. (canceled)

30. (canceled)

31. (canceled)

32. (canceled)

33. (canceled)

34. (canceled)

35. (canceled)

36. (canceled)

37. (canceled)

38. A conditional Gαs-knockout mouse comprising dendritic cells with a Gαs deletion.

39. The mouse of claim 38, wherein said mouse has a Th2 bias.

40. A transgenic Gαs-knockout mouse comprising dendritic cells with a Gαs deletion.

41. The mouse of claim 40, wherein Gαs deletion is a CD11c-specific deletion.

42. A cell comprising a Gαs deletion.

43. The cell of claim 42, wherein said cell is a murine cell.

44. (canceled)

45. (canceled)

46. (canceled)

47. A method of producing a Gαs-knockout mouse, said method comprising crossing a lox-flanked Gnas mouse with a CD11c-Cre or LysM-Cre mouse, wherein said Gαs-knockout mouse does not express Gαs.

48. The method of claim 47, wherein said Gαs-knockout mouse does not express Gαs in dendritic cells or macrophages.
